(19) United States
(12) Patent Application Publication
(10) Pub. No.: US 2026/0078168 A1
(43) Pub. Date: Mar. 19, 2026

(54) NON-ANIMAL BASED PROTEIN SOURCES WITH FUNCTIONAL PROPERTIES

(71) Applicant: Clara Foods Co., Daly City, CA (US)

(72) Inventors: Weixi ZHONG, Daly City, CA (US); Steve ROTHMAN, Daly City, CA (US); Aniket KALE, Daly City, CA (US); Ranjan PATNAIK, Daly City, CA (US); Joel Andrew KREPS, Daly City, CA (US); Kritika MAHADEVAN, Daly City, CA (US); Ying Joy ZHONG, Daly City, CA (US); Frank Douglas IVEY, Daly City, CA (US); Dariya IGNATENKO, Daly City, CA (US); Giulio SALERNO, Daly City, CA (US); Andrew MIYASHIRO, Daly City, CA (US)

(73) Assignee: Clara Foods Co., Daly City, CA (US)

(21) Appl. No.: 19/399,116

(22) Filed: Nov. 24, 2025

Related U.S. Application Data
(63) Continuation of application No. 18/045,422, filed on Oct. 10, 2022, now abandoned.
Application No. 18/045,422 is a continuation of application No. PCT/US22/76513, filed on Sep. 15, 2022.
(60) Provisional application No. 63/244,674, filed on Sep. 15, 2021.
(60) Provisional application No. 63/276,417, filed on Nov. 5, 2021.

(51) Int. Cl. C07K 14/77; A21D 2/26; A23J 3/04; A23J 3/22; A23L 33/17; C12G 3/04

(52) U.S. Cl. CPC C07K 14/77; A21D 2/262; A23J 3/04; A23J 3/225; A23L 33/17; C12G 3/04

(57) ABSTRACT

Provided herein are compositions with enhanced protein content, compositions with functional proteins, protein combinations and methods for the preparation thereof.

### CROSS-REFERENCE

This application is a continuation of U.S. patent application Ser. No. 18/045,422, filed Oct. 10, 2022, which is a continuation of International Patent Application No. PCT/US22/76513, filed Sep. 15, 2022, which claims the benefit of U.S. Provisional Patent Application No. 63/244,674, filed Sep. 15, 2021, and U.S. Provisional Patent Application No. 63/276,417, filed Nov. 5, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 10, 2022, is named 49160743301.xml and is 98,452 bytes in size.

### BACKGROUND OF THE INVENTION

Proteins are important dietary nutrients and food ingredients. They can serve as a fuel source or as sources of amino acids, including the essential amino acids that cannot be synthesized by the body. The daily recommended intake of protein for healthy adults is 10% to 35% of a person's total calorie needs, and currently the majority of protein intake for most humans is from animal-based sources. In addition, proteins are used in a wide variety of foods and food ingredients. In many cases, these proteins are sourced from animals. With the world population growth and the coinciding growth in global food demand, there is a need to provide alternative sustainable, non-animal-based sources of proteins as useful source of protein for daily diet, food ingredients and food products.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### SUMMARY OF THE INVENTION

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

In some aspects, provided herein is a consumable composition. In some embodiments, the consumable composition may comprise a recombinant ovalbumin (rOVA) protein and a recombinant clipped ovalbumin (rcOVA) protein. In some embodiments, the rOVA protein is a single polypeptide molecule. In some embodiments, the rcOVA protein has a complex of two polypeptide molecules. In some embodiments, the ovalbumin content of the consumable composition may comprise at least 0.1% w/w rcOVA.

In some aspects, provided herein is a consumable composition that may comprise a recombinant ovalbumin (rOVA) protein and a recombinant fragmented ovalbumin (rfOVA) protein. In some embodiments, the rOVA protein has a continuous covalent backbone, a continuous amino acid backbone, or a continuous polypeptide chain. In some embodiments, the rfOVA protein may comprise at least two peptide fragments of the rOVA protein. In some embodiments, the ovalbumin content of the consumable composition may comprise at least 0.1% w/w rfOVA.

In some embodiments, the rcOVA may comprise two or more polypeptide molecules connected to each other via non-covalent bonds. In some embodiments, the two polypeptide molecules are connected to each other in a configuration similar to a configuration of an unclipped full-length native ovalbumin protein (nOVA) or to a native configuration of the rOVA protein. In some embodiments, the two or more polypeptide molecules comprise continuous amino acid backbone or a continuous covalent backbone.

In some embodiments, the rOVA and rcOVA have identical amino acid sequences. In some embodiments, the rcOVA protein has the same number of amino acids as the rOVA protein. In some embodiments, the rOVA may be a full-length ovalbumin protein.

In some embodiments, the rcOVA may be clipped at a cleavage site. In some embodiments, the rcOVA may be clipped at a serine protease cleavage site. In some embodiments, the cleavage site may be selected from the group consisting of Ala352-Ser353, Asp350-Ala351, and His22-Ala23. In some embodiments, the rcOVA may be clipped towards the protein's C-terminal. In some embodiments, the rcOVA may be clipped towards the protein's N-terminal. In some embodiments, the rcOVA consists of 1 to 40 fewer amino acids than full length rOVA or nOVA (native OVA).

In some embodiments, the rcOVA may comprise a first polypeptide and a second polypeptide that are connected via non-covalent bonds, wherein the loss of one or more amino acids relative to the rOVA or nOVA amino acid sequence may be located on the N-terminus of the second polypeptide molecule, wherein the first polypeptide may comprise portion of its amino acid sequence that may be identical to an N-terminal region of the rOVA or nOVA and the second polypeptide may comprise a portion of its amino acid sequence that may be identical to a C-terminal region of the rOVA or nOVA.

In some embodiments, the rcOVA may comprise a first polypeptide and a second polypeptide that are connected via non-covalent bonds, wherein the loss of one or more amino acids relative to the rOVA or nOVA amino acid sequences may be located on the C-terminus of the first polypeptide, wherein the first polypeptide may comprise portion of its amino acid sequence that may be identical to an N-terminal region of the rOVA or nOVA and the second polypeptide may comprise a portion of its amino acid sequence that may be identical to a C-terminal region of the rOVA or nOVA.

In some embodiments, the rcOVA has an amino acid sequence that may be from 95% to 100% identical to the amino acid sequence of nOVA. In some embodiments, the non-continuous domain of the rcOVA may be not at a terminus relative to rOVA or nOVA. In some embodiments, the rcOVA has an amino acid sequence that may be 96% identical to the amino acid sequence of nOVA. In some embodiments, the rcOVA has an amino acid sequence that may be 97% identical to the amino acid sequence of nOVA. In some embodiments, the rcOVA has an amino acid sequence that may be 98% identical to the amino acid sequence of nOVA. In some embodiments, the rcOVA has an amino acid sequence that may be 99% identical to the amino acid sequence of nOVA. In some embodiments, the rcOVA has an amino acid sequence that may be identical to the amino acid sequence of nOVA.

In some embodiments, the rcOVA and rOVA have different elasticity in a rheological test. In some embodiments, the rcOVA has reduced elasticity or higher viscoelasticity values as provided by the loss factor compared to rOVA in a rheological test.

In some embodiments, the ovalbumin content of the consumable composition may comprise at least 0.5% w/w rcOVA. In some embodiments, the ovalbumin content of the consumable composition may comprise at least 1% w/w rcOVA. In some embodiments, the ovalbumin content of the consumable composition may comprise at least 2% w/w rcOVA. In some embodiments, the ovalbumin content of the consumable composition may comprise at least 5% w/w rcOVA. In some embodiments, the ovalbumin content of the consumable composition may comprise at least 7% w/w rcOVA. In some embodiments, the ovalbumin content of the consumable composition may comprise at least 10% w/w rcOVA. In some embodiments, the ovalbumin content of the consumable composition may comprise at least 20% w/w rcOVA. In some embodiments, the ovalbumin content of the consumable composition may comprise at least 50% w/w rcOVA. In some embodiments, the ovalbumin content of the consumable composition may comprise at least 70% w/w rcOVA. In some embodiments, the ovalbumin content of the consumable composition may comprise at least 80% w/w rcOVA. In some embodiments, the ovalbumin content of the consumable composition may comprise at most 90% w/w rcOVA. In some embodiments, the ovalbumin content of the consumable composition may comprise at most 70% w/w rcOVA. In some embodiments, the ovalbumin content of the consumable composition may comprise at most 50% w/w rcOVA. In some embodiments, the ovalbumin content of the consumable composition may comprise at most 30% w/w rcOVA. In some embodiments, the ovalbumin content of the consumable composition may comprise at most 20% w/w rcOVA. In some embodiments, the ovalbumin content of the consumable composition may comprise at most 10% w/w rcOVA. In some embodiments, the ovalbumin content of the consumable composition may comprise at most 7% w/w rcOVA. In some embodiments, the ovalbumin content of the consumable composition may comprise at most 5% w/w rcOVA. In some embodiments, the ovalbumin content of the consumable composition may comprise at most 2% w/w rcOVA. In some embodiments, the ovalbumin content of the consumable composition may comprise at most 1% w/w rcOVA. In some embodiments, the ovalbumin content of the consumable composition may comprise at most 0.5% w/w rcOVA.

In some embodiments, the consumable composition may be a food product. In some embodiments, the food product has a hardness different for a hardness of a control food product, wherein the control food product may be substantially identical to the food product except the control food product may comprise only rOVA or native ovalbumin (nOVA) as its ovalbumin content. In some embodiments, the food product has a chewiness different than a chewiness of a control food product, wherein the control food product may be substantially identical to the food product except the control food product may comprise only rOVA or native ovalbumin (nOVA) as its ovalbumin content. In some embodiments, the food product has a texture different than a texture of a control food product, wherein the control food product may be substantially identical to the food product except the control food product may comprise only rOVA or native ovalbumin (nOVA) as its ovalbumin content. In some embodiments, the rcOVA provides to the food product at least one egg white characteristic selected from gelling, foaming, whipping, fluffing, binding, springiness, aeration, coating, film forming, emulsification, browning, thickening, texturizing, humectant, clarification, and cohesiveness.

In some embodiments, the ovalbumin content in the food product may be at least 1% w/w. In some embodiments, the ovalbumin content in the food product may be at least 2% w/w. In some embodiments, the ovalbumin content in the food product may be at least 5% w/w. In some embodiments, the ovalbumin content in the food product may be at least 10% w/w. In some embodiments, the ovalbumin content in the food product may be at most 8% w/w. In some embodiments, the ovalbumin content in the food product may be at most 7% w/w. In some embodiments, the ovalbumin content in the food product may be at most 5% w/w. In some embodiments, the ovalbumin content in the food product may be at most 2% w/w. In some embodiments, the ovalbumin content in the food product may be at most 1% w/w. In some embodiments, the ovalbumin content in the food product may be from 1 to 20% w/w. In some embodiments, the ovalbumin content in the food product may be at from 2% to 15% w/w.

In some embodiments, the consumable composition may be a powder composition. In some embodiments, the ovalbumin content may comprise at least 85% of the powdered consumable composition w/w. In some embodiments, the ovalbumin content may comprise at least 90% of the powdered consumable composition w/w. In some embodiments, the ovalbumin content may comprise at least 95% of the powdered consumable composition w/w.

In some embodiments, the composition may be a liquid composition. In some embodiments, the liquid composition may be a concentrate. In some embodiments, the liquid composition may comprise at least 50% rOVA (w/w of total protein or w/w of composition). In some embodiments, the liquid composition may comprise at least about 60%, at least about 65%, at least about 75%, at least about 80%, at least about 85%, or at least about 90% rOVA (w/w). In some embodiments, the pH of the liquid composition may be between about 3.5 and about 10.

In some embodiments, the rOVA may comprise an amino acid sequence of a duck OVA, an ostrich OVA, or a chicken OVA. In some embodiments, the rOVA may comprise an amino acid sequence of SEQ ID NO: 2 or SEQ ID NO: 1 or an amino acid sequence with at least 70% identity to SEQ ID NO: 2 or SEQ ID NO: 1. In some embodiments, the rOVA further includes an EAEA amino acid sequence (SEQ ID NO: 76) at its N-terminus.

In some embodiments, the rcOVA may comprise an amino acid sequence of a duck OVA, an ostrich OVA, or a chicken OVA. In some embodiments, the rcOVA may comprise an amino acid sequence of SEQ ID NO: 2 or SEQ ID NO: 1 or an amino acid sequence with at least 70% identity to SEQ ID NO: 2 or SEQ ID NO: 1. In some embodiments, the rcOVA further includes an EAEA amino acid sequence (SEQ ID NO: 76) at the N-terminus.

In some embodiments, the rcOVA may be produced by protease treatment of rOVA. In some embodiments, the protease may be native to the host cell. In some embodiments, the protease may be heterologous to the host cell. In some embodiments, the host cell may be genetically modified to overexpress the protease. In some embodiments, the protease treatment may be performed during fermentation process in which rOVA may be produced by the host cell or the protease treatment may be performed after the fermentation process where rOVA may be produced by the host cell.

In some embodiments, the protease acts on the rOVA within the host cell. In some embodiments, the protease may be secreted from the host cell and acts on the rOVA in the fermentation medium. In some embodiments, the protease treatment may be performed on a purified protein preparation may comprise rOVA. In some embodiments, the protease treatment may be performed under conditions that increase protease activity. In some embodiments, the protease may be PRB1. In some embodiments, the protease may be a serine protease. In some embodiments, the protease may be selected from the group consisting of PRB1, Thrombin, Tissue plasminogen activator, Plasmin, Trypsin and Neuropsin.

In some embodiments, the rcOVA may be produced by elastase treatment of rOVA. In some embodiments, the elastase may be native to the host cell. In some embodiments, the elastase may be heterologous to the host cell. In some embodiments, the host cell may be genetically modified to overexpress the elastase. In some embodiments, the elastase treatment performed during fermentation process in which rOVA may be produced by the host cell or the elastase treatment may be performed after the fermentation process where rOVA may be produced by the host cell. In some embodiments, the elastase acts on the rOVA within the host cell. In some embodiments, the elastase may be secreted from the host cell and acts on the rOVA in the fermentation medium. In some embodiments, the elastase treatment may be performed on a purified protein preparation may comprise rOVA. In some embodiments, the elastase treatment may be performed under conditions that increase elastase activity.

In some embodiments, a ratio of elastase to ovalbumin may be at least 1:1000, wherein the elastase may be at least 4 units/mg. In some embodiments, a ratio of elastase to ovalbumin may be from 1:1000 to 1:100,000 wherein the elastase has an activity of at least 4 units/mg. In some embodiments, the elastase treatment may be performed at a temperature from about 35° C. to about 40° C. In some embodiments, the elastase treatment may be performed in presence of a low salt phosphate buffer, optionally, 1-5 mM sodium phosphate or another equivalent salt. In some embodiments, the elastase treatment may be performed at a pH from 3.5 to 10. In some embodiments, the elastase treatment may be performed at a pH from 6-8. In some embodiments, the elastase treatment may be performed at a pH of 7. In some embodiments, the elastase treatment may be performed for 3 hours or less.

In some aspects, provided herein are methods of producing consumable composition, such as described herein. In some embodiments, the method may comprise modulating the amount of clipping of rOVA. In some embodiments, the method may comprise inhibiting activity of one or more proteases; wherein the one or more proteases are known to cleave ovalbumin.

In some embodiments, the protease activity may be inhibited in a fermentation medium in which rOVA may be produced by a host cell. In some embodiments, the one or more native proteases in the host cell are underexpressed. In some embodiments, the underexpression may be caused by a genetic modification. In some embodiments, the native host cell may be genetically modified to knock out the one or more proteases.

In some embodiments, the method may comprise using one or more protease inhibitors. In some embodiments, the one or more protease inhibitors are added to a fermentation medium. In some embodiments, the host cells producing rOVA are cultured in the fermentation medium may comprise the one or more protease inhibitors. In some embodiments, the host cell may be genetically modified to express or overexpress one or more protease inhibitors.

In some embodiments, the protease inhibitors are selected from the group consisting of: 4-(2-aminoethyl) benzenesulfonyl fluoride hydrochloride (AEBSF), Alpha-1 antitrypsin, Alpha 2-antiplasmin, Antithrombin, 3-(1-(cyclohexyl(methyl)carbamoyl)-1H-imidazol-4-yl)pyridine 1-oxide (BIA 10-2474), Cl-inhibitor, Camostat, Cospin, CU-2010, CU-2020, chymostatin, Kallistatin, Kazal domain inhibitors, Mammary serine protease inhibitor (Maspin), Methoxy arachidonyl fluorophosphonate, Microviridin, Myeloid and erythroid nuclear termination stage-specific protein, nafamostat mesylate, ovomucoid, ovo-inhibitor, Plasminogen activator inhibitor-1, Plasminogen activator inhibitor-2, phenylmethylsulfonyl fluoride (PMSF), Protein C inhibitor (SERPINA5), Protein Z-related protease inhibitor, SERPINA9, SERPINB1, SERPINB3, SERPINB4, SERPINB6, SERPINB7, SERPINB8, SERPINB9, SERPINB13, SERPINE2, SPINT1, Upamostat, and Uterine serpin.

In some aspects, provided herein is consumable composition, wherein the consumable composition comprises three recombinant polypeptides: a first polypeptide comprising a first fragment of ovalbumin, a second polypeptide comprising a second fragment of ovalbumin; and a third polypeptide comprising a full-length ovalbumin. In some embodiments, the first polypeptide and the second polypeptide are in a complex. In some embodiments, the number of amino acids in the first polypeptide and the number of amino acids in the second polypeptide add up to the number of amino acids as the third polypeptide. In various embodiments, the third polypeptide comprises an amino acid sequence of SEQ ID NO: 2 or SEQ ID NO: 1 or an amino acid sequence with at least 70% identity to SEQ ID NO: 2 or SEQ ID NO: 1.

Additionally, any composition, food product, ingredient, use, or method disclosed herein is applicable to any herein-disclosed composition, food product, ingredient, use, or method. In other words, any aspect or embodiment described herein can be combined with any other aspect or embodiment as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-B illustrate glycosylation patterns of native OVA and rOVA produced in P. pastoris respectively.

FIG. 2 illustrates heat coagulation and foaming properties of whole egg, egg white and native OVA solutions.

FIG. 3 illustrates heat coagulation and foaming properties of egg white and native OVA compared to rOVA.

FIG. 4A illustrates gel electrophoresis migration of glycosylated native and recombinant OVA. Also shown are deglycosylated recombinant OVA treated with EndoH and PNGaseF enzymes.

FIG. 4B illustrates a chromatogram depicting glycosylation patterns of rOVA produced in P. pastoris.

FIG. 5 illustrates gelation results before and after foaming of various OVA samples compared to egg white.

FIG. 6 illustrates foaming of rOVA and control samples in an alcohol-based drink.

FIGS. 7A-B illustrate protein gel (FIG. 7A) and Western Blot anti-OVA gel (FIG. 7B) of analyses of batches 1 to 3 of rOVA protein products.

FIGS. 8A-C illustrate SDS-PAGE gels analyses of various batches of rOVA protein products.

FIG. 9 illustrates heat load variation tested for the heat treatment of various samples.

FIGS. 10A-C illustrate the correlation between heat load and characteristics of the sample (FIG. 10A: denaturation; FIG. 10B: foam capacity; and FIG. 10C: hardness (gelation indicator).

FIGS. 11A-C illustrate the foam capacity, foam stability and gelation (hardness) of nOVA with recombination ovalbumin (rOVA) with and without heat damage, and with and without clipping. FIG. 11A: comparison of foam capacity; FIG. 11B: comparison of foam stability; and FIG. 11C: comparison of thermogelation indicated by hardness of the gel.

FIGS. 12A-L illustrate the analyses of the foam capacity and stability, gelation (hardness), cohesiveness, chewiness, springiness, and adhesiveness characteristics of batch #9 (sample 009) and batch #10 (sample 006) treated with different heat treatment conditions (Trials 1-5 (T1-T5)), in comparison to egg white protein.

FIG. 13A illustrates the ovalbumin structure with an illustrative clipping site highlighted with an arrow.

FIG. 13B illustrates a SDS-PAGE gel with various protein preparations comprising the full-length or clipped forms of rOVA.

FIGS. 14A-B illustrate data from an amplitude sweep of 0%, 50%, and 100% clipped protein dispersions in 20 mM phosphate buffer (pH 7).

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are compositions and methods of making compositions for non-animal-based sources of proteins which provide nutritional as well as functional properties to food ingredients and consumable products for ingestion by an animal, including a human, such as for daily diet, ingredients for human food and treats and for human and animal nutrition.

The compositions and methods provided herein contain fermentation-derived ovalbumin, produced through recombinant technology, i.e., a recombinant ovalbumin (rOVA). In some embodiments, the fermentation-derived recombinant ovalbumin (rOVA) produced herein is a rOVA mixture comprising one or more clipped forms of the rOVA. The compositions and methods for making compositions comprising the rOVA mixture can increase the protein content of a consumable or food ingredient, and also provide functional features for use in the preparation of food ingredients and consumable food products for animal and human ingestion.

In some embodiments, the rOVA mixture comprising one or more clipped forms of the rOVA (rcOVA) provides one or more functional characteristics such as of gelling, foaming, whipping, fluffing, binding, springiness, aeration, coating, film forming, emulsification, browning, thickening, texturizing, humectant, clarification, and cohesiveness. In some embodiments, the rOVA mixture comprising one or more clipped forms of the rOVA with such feature(s) can be a food ingredient that provides for production of an egg-less or animal-free food ingredient or food product.

Ovalbumin from chicken contains a serine protease cleavage site between positions Ala352 and Ser353. See, e.g., Biosci. Biotechnol. Biochem., 67 (4), 830-837 (2003); Nature 347, 99-102 (1990). Among the serine proteases, porcine pancreatic elastase is known to only cleave ovalbumin at the reactive center. See, Journal of Biological Chemistry 259, 14335-14336, (1984); Protein Science 4, 613-621(1995). Other serine proteases such as chymotrypsin and subtilisin BPN cleave OVA at multiple locations. It was illustrated that Edman degradation of the small fragment from elastase cleavage of OVA indicated a sequence of Ser-Val-Ser, which is consistent with proteolysis between Ala352 and Ser353. See The Journal of Biological Chemistry, 259 (23), 14335-14336). OVA that has been treated with elastase and clipped as referred to as a “nicked” form of ovalbumin (see Biosci. Biotechnol. Biochem., 67 (4), 830-837 (2003)), which describes a situation where the two fragments remain together in a complex for the protein native state and become separable once denatured.

As used herein “native” in the context of native egg white, native egg protein, native ovalbumin and native egg, refers to the egg white, egg protein, ovalbumin or whole egg, respectively, produced by an animal or collected from an animal, in particular an egg-laying animal such as a bird. The rOVA and/or rcOVA and compositions containing rOVA and/or rcOVA can be used in food ingredients and food products, such that the ingredient or product does not contain any native egg white, native egg protein, native ovalbumin or native egg. In some cases, the ingredients or food products made using rOVA and/or rcOVA do not include any egg-white proteins other than rOVA and/or rcOVA. The rOVA and/or rcOVA and compositions containing rOVA and/or rcOVA can be used in food ingredients and food products, such that the ingredient or product does not contain any animal products.

In some embodiments, the rOVA mixture (comprising both unclipped rOVA and rcOVA or rcOVA alone) can (alone or with other ingredients) substitute for the use of whole egg or egg white in the production of a food product. In various embodiments, an rOVA mixture may comprise, consist essentially of, or consist of clipped forms of rOVA. In some embodiments, the feature(s) provided by the rOVA mixture is substantially the same or better than the same characteristic provided by a native egg white or native egg. For example, the rOVA mixture and/or compositions containing the rOVA mixture can have gelling, foaming, whipping, fluffing, binding, springiness, aeration, coating, film forming, emulsification, browning, thickening, texturizing, preserving moisture (humectant), clarification, and cohesiveness, improved color, such as a whiter color, as compared to native egg white or native whole egg and compositions made with native egg white.

Clipped Forms of rOVA in a rOVA Mixture

In some aspects, provided herein are consumable compositions which comprise recombinant ovalbumin. Single-point proteolytic nicking (referred to here as “clipping”) of the serpin loop of recombinant ovalbumin expressed in a host cell results in unique physical and chemical properties as compared to “intact” recombinant ovalbumin. The term “truncated”, “clipped”, and “nicked” are used interchangeably herein. This difference is useful in commercial applications both as a pure sample and in a mixture. The recombinant clipped ovalbumin (rcOVA) can be quantified by chromatography. The term “clipped” ovalbumin or “rcOVA” may refer to a protein that maintains the three-dimensional structure of the protein, such as through non-covalent interactions; yet comprises a non-contiguous amino acid backbone. The non-contiguous amino acid backbone may be connected through non-covalent bonds or may a complex of two or more peptide chains. In preferred embodiments, the clipped form of the protein has an amino acid sequence identical to the native protein. In some cases, the rcOVA is a complex of two or more different polypeptide fragments. For instance, the rcOVA can form a complex of polypeptides that are ovalbumin fragments. In some cases, the number of amino acids in the two or more polypeptides in a complex add up to the number of amino acids as the third polypeptide. In some cases, the clipped form of the protein has at least 85%, 90%, 95%, 97%, or 99% sequence identity to the full-length or native protein.

In some embodiment, the consumable composition comprises a mixture of a recombinant ovalbumin (rOVA) protein and a recombinant clipped ovalbumin (rcOVA) protein. In some embodiments, the rOVA protein has a single polypeptide chain or a continuous covalent peptide backbone or a continuous amino acid backbone. In some embodiments, the rcOVA protein comprises a complex of more than one polypeptide chains. The rcOVA may comprise a non-continuous covalent peptide backbone or a non-continuous amino acid backbone.

In some embodiments, the rcOVA may comprise two or more polypeptide molecules connected to each other via non-covalent bonds. In some embodiments, the rcOVA may comprise two or more polypeptide molecules connected to each other in a configuration similar to a configuration of an unclipped full-length native ovalbumin protein (nOVA) or to a native configuration of the rOVA protein. In some embodiments, the rcOVA may comprise a complex of more than two polypeptide chains.

In some embodiments, the consumable composition comprises a recombinant ovalbumin (rOVA) protein and a recombinant fragmented ovalbumin (rfOVA or rcOVA) protein. In some embodiments, the rOVA protein has a single polypeptide chain. In some embodiments, the rcOVA or rfOVA protein comprises a complex of at least two peptide fragments of the rOVA protein.

In some embodiments, the rOVA mixture comprises 1, 2, 3, 4, 5, 6, 7, 8, 9, or 10 clipped forms of the rOVA. In a preferred embodiment, the rOVA mixture comprises 1 clipped form of the rOVA. In another embodiment, the rOVA mixture comprises 2 clipped forms of the rOVA. In yet another embodiment, the rOVA mixture comprises 3 clipped forms of the rOVA. In yet another embodiment, the rOVA mixture comprises 4 clipped forms of the rOVA. In yet another embodiment, the rOVA mixture comprises 5 clipped forms of the rOVA.

In some embodiments, when the rOVA is expressed or produced, a mixture of various forms of the rOVA protein is derived. In some embodiments, the rOVA mixture comprises at least about 10%, about 20%, about 30%, about 40%, about 50%, about 60%, about 70%, about 80%, or about 90% clipped form or forms of the rOVA (w/w of total forms of the rOVA; or w/w of total of full-length form and clipped forms of the rOVA). In some cases, the rOVA mixture comprises 100% clipped form or forms of the rOVA (w/w). In some embodiments, the rOVA mixture comprises about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% clipped form or forms of the rOVA (w/w). In some embodiments, the rOVA mixture comprises about 10%, about 20%, about 30%, about 40%, about 50%, about 60%, about 70%, about 80%, about 90% or about 100% clipped form or forms of the rOVA (w/w). In some embodiments, the rOVA mixture comprises about 0%-10%, about 10%-20%, about 20%-30%, about 30%-40%, about 40%-50%, about 50%-60%, about 60%-70%, about 70%-80%, about 80%-90%, or about 90%-100% clipped form or forms of the rOVA (w/w).

In some embodiments, the ovalbumin content of the consumable composition comprises 0.1% w/w to 30% w/w rcOVA while the rest of the ovalbumin is rOVA (full-length ovalbumin with a single peptide chain). In some embodiments, the ovalbumin content of the consumable composition comprises at least 0.1% w/w rcOVA. In some embodiments, the ovalbumin content of the consumable composition comprises at most 30% w/w rcOVA. In some embodiments, the ovalbumin content of the consumable composition comprises 0.1% w/w to 0.5% w/w, 0.1% w/w to 1% w/w, 0.1% w/w to 5% w/w, 0.1% w/w to 10% w/w, 0.1% w/w to 15% w/w, 0.1% w/w to 20% w/w, 0.1% w/w to 30% w/w, 0.5% w/w to 1% w/w, 0.5% w/w to 5% w/w, 0.5% w/w to 10% w/w, 0.5% w/w to 15% w/w, 0.5% w/w to 20% w/w, 0.5% w/w to 30% w/w, 1% w/w to 5% w/w, 1% w/w to 10% w/w, 1% w/w to 15% w/w, 1% w/w to 20% w/w, 1% w/w to 30% w/w, 5% w/w to 10% w/w, 5% w/w to 15% w/w, 5% w/w to 20% w/w, 5% w/w to 30% w/w, 10% w/w to 15% w/w, 10% w/w to 20% w/w, 10% w/w to 30% w/w, 15% w/w to 20% w/w, 15% w/w to 30% w/w, or 20% w/w to 30% w/w rcOVA. In some embodiments, the ovalbumin content of the consumable composition comprises about 0.1% w/w, 0.5% w/w, 1% w/w, 5% w/w, 10% w/w, 15% w/w, 20% w/w, or 30% w/w rcOVA. In some embodiments, the ovalbumin content of the consumable composition comprises at least 0.1% w/w, 0.5% w/w, 1% w/w, 5% w/w, 10% w/w, 15% w/w, 20% w/w, or 30% w/w rcOVA. In some embodiments, the ovalbumin content of the consumable composition comprises at most 0.1% w/w, 0.5% w/w, 1% w/w, 5% w/w, 10% w/w, 15% w/w, 20% w/w, or 30% w/w rcOVA.

In some embodiments, the ovalbumin content of the consumable composition comprises a high concentration of rcOVA, for instance from 35% w/w to 100% w/w rcOVA. In some embodiments, the ovalbumin content of the consumable composition comprises at least 35% w/w rcOVA. In some embodiments, the ovalbumin content of the consumable composition comprises 100% w/w rcOVA. In some embodiments, the ovalbumin content of the consumable composition comprises 35% w/w to 40% w/w, 35% w/w to 50% w/w, 35% w/w to 70% w/w, 35% w/w to 80% w/w, 35% w/w to 90% w/w, 35% w/w to 100% w/w, 40% w/w to 50% w/w, 40% w/w to 70% w/w, 40% w/w to 80% w/w, 40% w/w to 90% w/w, 40% w/w to 100% w/w, 50% w/w to 70% w/w, 50% w/w to 80% w/w, 50% w/w to 90% w/w, 50% w/w to 100% w/w, 70% w/w to 80% w/w, 70% w/w to 90% w/w, 70% w/w to 100% w/w, 80% w/w to 90% w/w, 80% w/w to 100% w/w, or 90% w/w to 100% w/w rcOVA. In some embodiments, the ovalbumin content of the consumable composition comprises about 35% w/w, 40% w/w, 50% w/w, 70% w/w, 80% w/w, 90% w/w, or 100% w/w rcOVA. In some embodiments, the ovalbumin content of the consumable composition comprises at least 35% w/w, 40% w/w, 50% w/w, 70% w/w, 80% w/w, or 90% w/w rcOVA. In some embodiments, the ovalbumin content of the consumable composition comprises at most 35% w/w, 40% w/w, 50% w/w, 70% w/w, 80% w/w, 90% w/w rcOVA.

In some embodiments, the rOVA mixture does not comprise full-length form of the rOVA. In some embodiments, the rOVA mixture consists of clipped form or forms of the rOVA. In some embodiments, the rOVA mixture consists essentially of clipped form or forms of the rOVA. In some embodiments, the rOVA mixture comprises the fragment of the rOVA that has been clipped (e.g., by a protease) from a full-length rOVA. In one embodiment, the rOVA mixture comprises the clipped form of the rOVA and the fragment of the rOVA that has been clipped (e.g., by a protease) from a full-length rOVA. In some embodiments, the clipped form of the rOVA and the fragment of the rOVA that has been clipped (e.g., by a protease) from a full-length rOVA remain together in a complex for the protein native state, but become separable once they are denatured.

In some cases, an elastase enzyme truncates or clips the full-length OVA protein. In some cases, a subtilisin enzyme (for instance, subtilisin carlsberg of Bacillus lichenformis) truncates or clips the full-length OVA protein. In some cases, a pepsin enzyme truncates or clips the full-length OVA protein.

Endogenous protease activity can be enhanced or inhibited by altering the manufacturing conditions to increase endogenous protease concentration or activity. Some examples of such conditions are provided in Fujishiro (1980), the contents of which is incorporated herein by reference in its entirety.

In some embodiments, a host cell may be engineered to over-express a native protease such as PRB1. In some embodiments, the host cell may be engineered to express or over-express a protease heterologous to the host cell. The proteases expressed or overexpressed may be serine proteases. Examples of proteases include PRB1, Thrombin, tissue plasminogen activator (tPA), plasmin, trypsin, and neuropsin. In some embodiments, more than one proteases may be expressed or overexpressed in the host cell which expresses recombinant ovalbumin. The expression or overexpression of proteases may be performed to modulate the amount of clipped ovalbumin produced during fermentation. For instance, a host cell may overexpress one or more proteases to increase the amount of clipped OVA produced during fermentation.

In some embodiments, a host cell may be engineered to over-express a native elastase. In some embodiments, the host cell may be engineered to express or over-express an elastase heterologous to the host cell. In some embodiments, more than one elastases may be expressed or overexpressed in the host cell which expresses recombinant ovalbumin. The expression or overexpression of elastases may be performed to modulate the amount of clipped ovalbumin produced during fermentation. For instance, a host cell may overexpress one or more elastases to increase the amount of clipped OVA produced during fermentation.

Protease inhibitors may be utilized to modulate the amount of clipped ovalbumin produced during fermentation. Protease inhibitors may be added to the fermentation medium while recombinant ovalbumin is being produced by a host cell. Alternatively, protease inhibitors may be used to modulate the amount of clipped ovalbumin in a purified ovalbumin protein preparation.

Illustrative protease inhibitors may include but are not limited to: 4-(2-aminoethyl) benzenesulfonyl fluoride hydrochloride (AEBSF), Alpha-1 antitrypsin, Alpha 2-antiplasmin, Antithrombin, 3-(1-(cyclohexyl(methyl)carbamoyl)-1H-imidazol-4-yl)pyridine 1-oxide (BIA 10-2474), Cl-inhibitor, Camostat, Cospin, CU-2010, CU-2020, chymostatin, Kallistatin, Kazal domain inhibitors, Mammary serine protease inhibitor (Maspin), Methoxy arachidonyl fluorophosphonate, Microviridin, Myeloid and erythroid nuclear termination stage-specific protein, nafamostat mesylate, ovomucoid, ovo-inhibitor, Plasminogen activator inhibitor-1, Plasminogen activator inhibitor-2, phenylmethylsulfonyl fluoride (PMSF), Protein C inhibitor (SERPINA5), Protein Z-related protease inhibitor, SERPINA9, SERPINB1, SERPINB3, SERPINB4, SERPINB6, SERPINB7, SERPINB8, SERPINB9, SERPINB13, SERPINE2, SPINT1, Upamostat, and Uterine serpin.

In some embodiments, a clipped recombinant ovalbumin (rcOVA) can be produced by addition of exogenous elastase and/or by amplification of endogenous serine protease of the host cell during or after the manufacturing process (e.g., during fermentation and expression of the rOVA or during downstream processing steps). For instance, the predominant serine protease in Pichia species is PRB1 (also referred to as Proteinase B). The site of endogenous proteolytic cleavage in Pichia is consistent with the peptide-cleavage handle motif established for elastase. In some cases, the clipping of ovalbumin can be achieved by addition of exogenous elastase (>4 units/mg) at a ratio within an order of 1 part elastase to 10,000 parts ovalbumin (mass/mass). The clipping of rOVA may be performed at 37° C. in low-salt phosphate buffer near neutral pH. Clipping may be performed within less than 48 hours. Other different time and temperature conditions extrapolated from the above conditions may be utilized for rOVA clipping. In some cases, salt, pH, and other environmental conditions may be modified to increase elastase and ovalbumin stability and activity.

In some embodiments, the protease treatment of rOVA is performed at a protease to OVA ratio of at least 1:100,000. In some embodiments, the protease treatment of rOVA is performed at a protease to OVA ratio of at most 1:50. In some embodiments, the protease treatment of rOVA is performed at a protease to OVA ratio of at about 1:50, 1:100, 1:1,000, 1:2,000, 1:5,000, 1:10,000 1:20,000, 1:50,000, 1:100,000. In some embodiments, the protease treatment of rOVA is performed at a protease to OVA ratio from 1:50 to 1:1,000, 1:50 to 1:10,000, 1:50 to 1:100,000, 1:100 to 1:1,000, 1:100 to 1:10,000, 1:100 to 1:50,000, 1:100 to 1:100,000, 1:1,0000 to 1:10,000, 1:1,000 to 1:50,000, 1:1,000 to 1:100,000, 1:10,000 to 1:50,000, 1:10,000 to 1:100,000. In some cases, the protease may be an elastase.

In some embodiments, the protease treatment of rOVA is performed at a temperature of 32° C. to 40° C. In some embodiments, the protease treatment of rOVA is performed at a temperature of at least 32° C. In some embodiments, the protease treatment of rOVA is performed at a temperature of at most 40° C. In some embodiments, the protease treatment of rOVA is performed at a temperature of 32° C. to 34° C., 32° C. to 35° C., 32° C. to 37° C., 32° C. to 40° C., 34° C. to 35° C., 34° C. to 37° C., 34° C. to 40° C., 35° C. to 37° C., 35° C. to 40° C., or 37° C. to 40° C. In some embodiments, the protease treatment of rOVA is performed at a temperature of about 32° C., 34° C., 35° C., 37° C., or 40° C.

In some embodiments, the protease treatment of rOVA is performed at a pH of 5 to 9. In some embodiments, the protease treatment of rOVA is performed at a pH of at least 5. In some embodiments, the protease treatment of rOVA is performed at a pH of at most 9. In some embodiments, the protease treatment of rOVA is performed at a pH of 5 to 6, 5 to 7, 5 to 8, 5 to 9, 6 to 7, 6 to 8, 6 to 9, 7 to 8, 7 to 9, or 8 to 9. In some embodiments, the protease treatment of rOVA is performed at a pH of about 5, 6, 7, 8, or 9.

In some embodiments, the protease treatment of rOVA is performed for 0.5 hours to 3 hours. In some embodiments, the protease treatment of rOVA is performed for at least 0.5 hours. In some embodiments, the protease treatment of rOVA is performed for at most 3 hours. In some embodiments, the protease treatment of rOVA is performed for 0.5 hours to 0.7 hours, 0.5 hours to 1 hours, 0.5 hours to 1.2 hours, 0.5 hours to 1.5 hours, 0.5 hours to 1.7 hours, 0.5 hours to 2 hours, 0.5 hours to 2.5 hours, 0.5 hours to 3 hours, 0.7 hours to 1 hours, 0.7 hours to 1.2 hours, 0.7 hours to 1.5 hours, 0.7 hours to 1.7 hours, 0.7 hours to 2 hours, 0.7 hours to 2.5 hours, 0.7 hours to 3 hours, 1 hours to 1.2 hours, 1 hours to 1.5 hours, 1 hours to 1.7 hours, 1 hours to 2 hours, 1 hours to 2.5 hours, 1 hours to 3 hours, 1.2 hours to 1.5 hours, 1.2 hours to 1.7 hours, 1.2 hours to 2 hours, 1.2 hours to 2.5 hours, 1.2 hours to 3 hours, 1.5 hours to 1.7 hours, 1.5 hours to 2 hours, 1.5 hours to 2.5 hours, 1.5 hours to 3 hours, 1.7 hours to 2 hours, 1.7 hours to 2.5 hours, 1.7 hours to 3 hours, 2 hours to 2.5 hours, 2 hours to 3 hours, or 2.5 hours to 3 hours. In some embodiments, the protease treatment of rOVA is performed for 0.5 hours, 0.7 hours, 1 hours, 1.2 hours, 1.5 hours, 1.7 hours, 2 hours, 2.5 hours, or 3 hours.

In some embodiments, the molecular weight of the clipped form of the rOVA is at least about 40%, about 50%, about 60%, about 70%, about 80%, or about 90% of the molecular weight of the full-length rOVA. In some embodiments, the molecular weight of the clipped form of the rOVA is about 40%, 45%, 50%, 55%, 60%, 65%, 70%, 75%, 80%, 85%, 90%, or 95% of the molecular weight of the full-length rOVA. In some embodiments, the molecular weight of the clipped form of the rOVA is about 90%, 91%, 92%, 93%, 94%, 95%, 96%, 97%, 98%, 99%, or 100% of the molecular weight of the full-length rOVA.

In some embodiments, the clipped form of the rOVA is of at least 50%, 55%, 60%, 65%, 70%, 75%, 80%, 85%, 90%, or 95% sequence identity of the full-length rOVA. In some embodiments, the clipped form of the rOVA is of at least about 90%, 91%, 92%, 93%, 94%, 95%, 96%, 97%, 98%, or 99% sequence identity of the full-length rOVA. Preferably, the clipped form of the rOVA has at least 99% of the molecular weight of the full-length rOVA; more preferably, 100% of the molecular weight of the full-length rOVA.

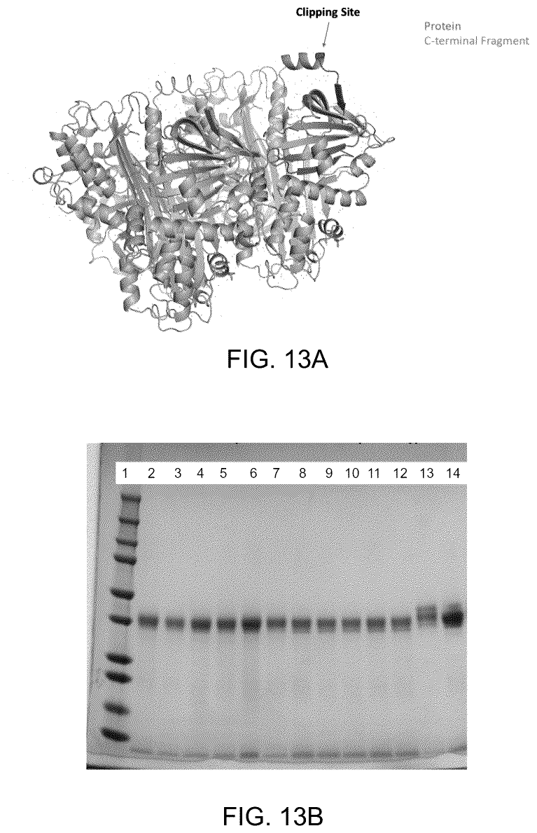

In some embodiments, the clipped form of the rOVA results from clipping towards the N-terminus of the rOVA. In some embodiments, the clipped form of the rOVA results from clipping towards the C-terminus of the rOVA. The rOVA may be clipped by a serine protease towards the C-terminal, thereby creating a distinct C-terminal fragment, an example of which is shown in FIG. 13A. In some embodiments, the rOVA and rcOVA have identical amino acid sequences. In some embodiments, the rcOVA protein has the same number of amino acids as the rOVA protein.

In some embodiments, the rOVA is clipped by a serine protease. In a specific embodiment, the rOVA is clipped at a serine cleavage site between an Ala and a Ser.

In a specific embodiment, the rOVA is clipped at a cleavage site at Ala352(P1)-Ser353(P1′) in SEQ ID NO: 75. In a specific embodiment, the rOVA is clipped from amino acids 346-352 in SEQ ID NO: 75. In a specific embodiment, the rOVA is clipped at a cleavage site at Asp350(P1)-Ala351(P1′) in SEQ ID NO: 75. In a specific embodiment, the rOVA is clipped at a cleavage site at His22(P1)-Ala23(P1′) in SEQ ID NO: 75.

In some embodiments, the rcOVA is clipped towards the protein's C-terminal. In some embodiments, the rcOVA is clipped towards the protein's N-terminal.

Food Ingredients and Food Products with rOVA Mixture Comprising One or More Clipped Forms of the rOVA

Food ingredients and food products disclosed herein include compositions comprise a rOVA mixture that comprise, consists essentially of, or consist of clipped forms of rOVA, where the rOVA mixture provides at least one functional feature to the composition, food ingredient, or food product. In some cases, at least one functional feature provided by the rOVA mixture is comparable or substantially similar to a native egg or egg white or native OVA (nOVA). For instance, it may provide any one of gelling, foaming, whipping, fluffing, binding, springiness, aeration, coating, film forming, emulsification, browning, thickening, texturizing, preserving moisture (humectant), clarification, and cohesiveness comparable to a whole egg, egg-white or nOVA composition. In some embodiments, the at least one functional feature is provided by or provided substantially by the inclusion of the rOVA mixture in the food ingredient or food product, for example, in the absence of any other whole egg proteins or egg white proteins.

Such compositions can include the rOVA mixture in an amount between 0.1% and 25% on a weight/weight (w/w) or weight/volume (w/v) basis. In some embodiments, the rOVA mixture may be present at or at least at 0.1%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% on a weight/weight (w/w) or weight/volume (w/v) basis. These concentrations can be based on the dry weight of the composition. Additionally, or alternatively, the concentration of the rOVA mixture in such compositions is at most 30%, 20%, 15%, 10%, 5%, 4%, 3%, 2% or 1% on a w/w or w/v basis. In some embodiments, the rOVA mixture in the food ingredient or food product can be at a concentration range of 0.1%-20%, 1%-20%, 0.1%-10%, 1%-10%, 0.1%-5%, 1%-5%, 2-10%, 4-8%, 4-10%, 4-12%, 0.1%-2%, 1%-2% or 0.1-10%.

Provided herein are consumable food compositions and methods of making such compositions where the rOVA mixture provided herein provides at least one feature of whole egg or egg-whites to a consumable food composition. In some embodiments, the rOVA mixture is added to a consumable food composition to increase the protein content, such as for added nutrition. In some embodiments, the rOVA mixture is present in the consumable food composition between about 1% and about 40% on a weight per total weight (w/w) and/or weight per total volume (w/v) of composition basis. For example, in a composition of 100 ml, the rOVA mixture comprising one or more forms of the rOVA is present at 30 g and the rOVA mixture is thus at a 30% concentration (w/v) or for example, in a composition of 100 g, thus the rOVA mixture is present at 30 g and the rOVA is thus at a 30% concentration (w/w). In some embodiments, the concentration of the rOVA mixture is or is about 0.5%, %, 40%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% on a w/w and/or w/v of composition basis. In some embodiments, the rOVA mixture is present at a concentration of or of about 0.5-1%, 1-5%, 2-8%, 4-8%, 2-12%, 4-12%, 5-10%, 10-15%, 15-20%, 20-25%, 25-30% or the rOVA mixture is present concentration greater than 1%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% w/w and/or w/v.

A consumable product can include one or more other proteins, such as a non-OVA proteins or non-recombinant proteins (e.g., a vegetarian protein or vegan protein). The rOVA mixture can increase amount of protein content in a consumable product, and/or provide one or more egg-white like features. For example, the consumable composition can include a whey protein, a pea protein, a soy protein, an almond protein, an oat protein, a flax seed protein, a vegetable protein, or an egg-white protein. The consumable protein may include an extruded plant protein or a non-extruded plant protein. In some cases, the one or more other proteins can comprise OVA having an amino acid sequence naturally found in a bird or a reptile. In some embodiments, the rOVA mixture provides the only egg-derived or egg-related protein to a consumable product.

In some embodiments, the compositions and methods for making compositions have an egg-white like property and increase the protein content in the composition. In some embodiments, the compositions and methods for making compositions with an egg-white like property increase the protein content, while not adversely affecting the stability, or one or more sensory qualities of the composition.

In some embodiments, the consumable food compositions and methods for making consumable food compositions comprise the rOVA mixture comprising one or more clipped forms of the rOVA and the addition of the rOVA mixture generates an egg-white like composition. The consumable food composition may be a finished product or an ingredient for making a finished product, e.g., a liquid or a powdered rOVA composition comprising a rOVA mixture comprising one or more clipped forms of the rOVA.

In some embodiments, the rOVA mixture may be used on its own or in combination with other components to form a composition. In some embodiments, the rOVA mixture is used as an ingredient to form a composition and the rOVA ingredient (or rOVA starting composition to be added) may contain about or at least about 10%, 20%, 30%, 40%, 50%, 55%, 60%, 65%, 70%, 75%, 80%, 85%, 90%, 95%, 96%, 97%, 98%, or 99% full length or clipped forms of the rOVA by weight per total weight (w/w) and/or weight per total volume (w/v). In some cases, the rOVA mixture comprises nearly 100% clipped form or forms of the rOVA (w/v). In some cases, the rOVA mixture comprises 100% full-length rOVA (w/v) with little amounts of rcOVA. In some cases, a composition described herein may contain up to about 10%, 20%, 30%, 40%, 50%, 55%, 60%, 65%, 70%, 75%, 80%, 85%, 90%, 95%, 96%, 97%, 98%, or 99% full length or clipped forms of the rOVA by w/w or w/v. In some embodiments, about or at least about 10%, 20%, 30%, 40%, 50%, 55%, 60%, 65%, 70%, 75%, 80%, 85%, 90%, 95%, 96%, 97%, 98%, or 99% of the protein in a composition is full length or clipped forms of the rOVA by weight per total weight (w/w) and/or weight per total volume (w/v). In some cases, the rOVA mixture comprises 100% clipped form or forms of the rOVA (w/v). In some cases, up to or about 10%, 20%, 30%, 40%, 50%, 55%, 60%, 65%, 70%, 75%, 80%, 85%, 90%, 95%, 96%, 97%, 98%, or 99% of the protein in a composition is full length or clipped forms of the rOVA by w/w or w/v.

In some embodiments, the composition described herein contains total protein at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, or 75 g total protein per 100 mL liquid (e.g., water or other consumable liquid). In some cases, the composition described herein contains total protein at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, 75, 80, 85, 90, 95, or 100 g total protein per 100 g composition (e.g., powder).

In some embodiments, the composition described herein contains the rOVA mixture at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, or 75 g per 100 mL liquid (e.g., water or other consumable liquid). In some cases, a composition described herein contains the rOVA mixture at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, 75, 80, 85, 90, 95, or 100 g total protein per 100 g composition (e.g., powder).

In some embodiments, a composition described herein contains total protein at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5.0 g total protein per 100 mL liquid (e.g., water or other consumable liquid). In some cases, a composition described herein contains total protein at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5.0 g total protein per 100 g composition (e.g., powder).

In some embodiments, a composition described herein contains the rOVA mixture at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5.0 g per 100 mL liquid (e.g., water or other consumable liquid). In some cases, a composition described herein contains rOVA at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5.0 g per 100 g composition (e.g., powder).

In some embodiments, the rOVA-containing consumable composition is a liquid composition. In such cases, the concentration of the rOVA mixture in the liquid composition may be between 0.1% to 90% in weight per total volume (w/v). In some embodiments, the concentration of the rOVA mixture in the liquid composition may be at least 0.1% w/v. In some embodiments, the concentration of the rOVA mixture in the liquid composition may be at most 90% w/v. In some embodiments, the concentration of the rOVA mixture in the liquid composition may be from 0.1% to 1%, 0.1% to 5%, 0.1% to 10%, 0.1% to 15%, 0.1% to 20%, 0.1% to 25%, 0.1% to 30%, 0.1% to 35%, 0.1% to 40%, 1% to 5%, 1% to 10%, 1% to 15%, 1% to 20%, 1% to 25%, 1% to 30%, 1% to 35%, 1% to 40%, 5% to 10%, 5% to 15%, 5% to 20%, 5% to 25%, 5% to 30%, 5% to 35%, 5% to 40%, 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 20% to 40%, 25% to 30%, 25% to 35%, 25% to 40%, 30% to 35%, 30% to 40%, 35% to 40%, 40% to 45%, 45% to 50%, 50% to 55%, 55% to 60%, 60% to 65%, 65% to 70%, 70% to 75%, 75% to 80%, 80% to 85%, 85% to 90%, or 90% to 95% in w/v. The concentration of the rOVA mixture in the liquid composition may be about 0.1%, 1%, 5%, 10%, 15%, 20%, 25%, 30%, 35%, 40%, 45%, 50%, 55%, 60%, 65%, 70%, 75%, 80%, 85%, 90%, or 95% w/v. The concentration of the rOVA mixture in the liquid composition may be at least 0.1%, 1%, 5%, 10%, 15%, 20%, 25%, 30%, 35%, 40%, 45%, 50%, 55%, 60%, 65%, 70%, 75%, 80%, 85%, 90%, or 95% w/v. The concentration of the rOVA mixture in the liquid composition may be at most 1%, 5%, 10%, 15%, 20%, 25%, 30%, 35% 40%, 45%, 50%, 55%, 60%, 65%, 70%, 75%, 80%, 85%, 90%, or 95% w/v. In some embodiments, the rOVA mixture is the sole protein in the liquid composition. In other embodiments, the liquid composition comprises proteins other than full length or clipped forms of rOVA.

In some embodiments, the rOVA-containing consumable composition is a solid composition. In such cases, the concentration of the rOVA mixture in the solid composition may be between 0.1% to 70% weight per total weight (w/w) and/or weight per total volume (w/v). In some embodiments, the concentration of the rOVA mixture in the solid composition may be at least 0.1% w/w or w/v. In some embodiments, the concentration of the rOVA mixture in the solid composition may be at most 70% w/w or w/v. In some embodiments, the concentration of the rOVA mixture in the solid composition may be 0.1% to 1%, 0.1% to 10%, 0.1% to 20%, 0.1% to 30%, 0.1% to 40%, 0.1% to 50%, 0.1% to 60%, 0.1% to 70%, 1% to 10%, 1% to 20%, 1% to 30%, 1% to 40%, 1% to 50%, 1% to 60%, 1% to 70%, 10% to 20%, 10% to 30%, 10% to 40%, 10% to 50%, 10% to 60%, 10% to 70%, 20% to 30%, 20% to 40%, 20% to 50%, 20% to 60%, 20% to 70%, 30% to 40%, 30% to 50%, 30% to 60%, 30% to 70%, 40% to 50%, 40% to 60%, 40% to 70%, 50% to 60%, 50% to 70%, or 60% to 70% w/w or w/v. In some embodiments, the concentration of the rOVA mixture in the solid composition may be 0.1%, 1%, 10%, 20%, 30%, 40%, 50%, 60%, or 70% w/w or w/v. In some embodiments, the concentration of the rOVA mixture in the solid composition may be at least 0.1%, 1%, 10%, 20%, 30%, 40%, 50% or 60% w/w or w/v. In some embodiments, the concentration of the rOVA mixture in the solid composition may be at most 1%, 10%, 20%, 30%, 40%, 50%, 60%, or 70% w/w or w/v.

In some embodiments, the rOVA-containing consumable composition is a powdered composition. In such cases, the concentration of the rOVA mixture in the powder composition may be between 15% to 99% weight per total weight (w/w) and/or weight per total volume (w/v). In some embodiments, the concentration of the rOVA mixture in the powder composition may be at least 15% w/w or w/v. In some embodiments, the concentration of the rOVA mixture in the powder composition may be at most 99% w/w or w/v. In some embodiments, the concentration of the rOVA mixture in the powder composition may be 15% to 30%, 15% to 45%, 15% to 60%, 15% to 75%, 15% to 80%, 15% to 85%, 15% to 90%, 15% to 95%, 15% to 99%, 30% to 45%, 30% to 60%, 30% to 75%, 30% to 80%, 30% to 85%, 30% to 90%, 30% to 95%, 30% to 99%, 45% to 60%, 45% to 75%, 45% to 80%, 45% to 85%, 45% to 90%, 45% to 95%, 45% to 99%, 60% to 75%, 60% to 80%, 60% to 85%, 60% to 90%, 60% to 95%, 60% to 99%, 75% to 80%, 75% to 85%, 75% to 90%, 75% to 95%, 75% to 99%, 80% to 85%, 80% to 90%, 80% to 95%, 80% to 99%, 85% to 90%, 85% to 95%, 85% to 99%, 90% to 95%, 90% to 99%, or 95% to 99% w/w or w/v. In some embodiments, the concentration of the rOVA mixture in the powder composition may be about 15%, 30%, 45%, 60%, 75%, 80%, 85%, 90%, 95%, or 99% w/w or w/v. In some embodiments, the concentration of the rOVA in the powder composition may be at least 15%, 30%, 45%, 60%, 75%, 80%, 85%, 90% or 95% w/w or w/v. In some embodiments, the concentration of the rOVA mixture in the powder composition may be at most 30%, 45%, 60%, 75%, 80%, 85%, 90%, 95%, or 99% w/w or w/v. In some embodiments, the powder composition does not comprise proteins other than full length or clipped forms of rOVA. In other embodiments, the powder composition comprises proteins other than full length or clipped forms of rOVA.

In some cases, the powder composition provided herein may be a concentrate which comprises at least 70% full length or clipped forms of rOVA w/w. In some cases, the powder composition may be a concentrate which comprises at least 80% full length or clipped forms of rOVA w/w. In some cases, the powder composition may be an isolate which comprises at least full length or clipped forms of 90% rOVA w/w. In some cases, the powder composition may be an isolate which comprises at least 95% full length or clipped forms of rOVA w/w.

In some embodiments, the rOVA-containing consumable composition is a concentrated liquid composition. In such cases, the concentration of the rOVA mixture in the concentrated liquid composition may be between 10% to 60% weight per total weight (w/w) and/or weight per total volume (w/v). In some embodiments, the concentration of the rOVA mixture in the concentrated liquid may be at least 10% w/w or w/v. In some embodiments, the concentration of the rOVA mixture in the concentrated liquid may be at most 60% w/w or w/v. In some embodiments, the concentration of the rOVA mixture in the concentrated liquid may be 10% to 20%, 10% to 30%, 10% to 40%, 10% to 50%, 10% to 60%, 20% to 30%, 20% to 40%, 20% to 50%, 20% to 60%, 30% to 40%, 30% to 50%, 30% to 60%, 40% to 50%, 40% to 60%, or 50% to 60% w/w or w/v. In some embodiments, the concentration of the rOVA mixture in the concentrated liquid may be about 10%, 20%, 30%, 40%, 50%, or 60% w/w or w/v. In some embodiments, the concentration of the rOVA mixture in the concentrated liquid may be at least 10%, 20%, 30%, 40% or 50% w/w or w/v. In some embodiments, the concentration of the rOVA mixture in the concentrated liquid may be at most 20%, 30%, 40%, 50%, or 60% w/w or w/v. In some embodiments, the liquid may include any consumable solvent, e.g., water, fruit or vegetable juice, fruit or vegetable extract, fruit or vegetable paste, broth, sauce, dairy, oil, or other cooking base.

In some embodiments, the rOVA-containing consumable composition is a prepared food for example, as a baked good, a salad dressing, an egg-like dish (such as an egg-patty or scramble), a dessert or dairy-like product or a meat-analog (such as a vegan meat patty, sausage or hot dog). Such compositions can comprise the rOVA mixture in an amount between 0.1% and 20% on a weight/weight (w/w) or weight/volume (w/v) basis. In some embodiments, the rOVA mixture may be present at or at least at 0.1%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20% on a weight/weight (w/w) or weight/volume (w/v) basis. Additionally, or alternatively, the concentration of the rOVA mixture in such compositions is at most 30%, 20%, 15%, 10%, 5%, 4%, 3%, 2% or 1% on a w/w or w/v basis. In some embodiments, the rOVA mixture in the food ingredient or food product can be at a concentration range of 0.1%-20%, 1%-20%, 0.1%-10%, 1%-10%, 0.1%-5%, 1%-5%, 0.1%-2%, 1%-2% or 0.1-1%.

Features and Characteristics of rOVA Compositions and Food Ingredients and Food Products Containing rOVA Mixture Comprising Clipped Forms of rOVA

The rOVA containing compositions herein can provide one or more functional features to food ingredients and food products. In some embodiments, the rOVA provides a nutritional feature such as protein content, protein fortification and amino acid content to a food ingredient or food product. The nutritional feature provided by rOVA in the composition may be comparable or substantially similar to an egg, egg white or native OVA (nOVA). The nutritional feature provided by rOVA in the composition may be better than that provided by a native whole egg or native egg white. In some cases, rOVA provides the one or more functional features of egg-white in absence of any other egg-white proteins.

In some embodiments, the rOVA mixture also provides a nutritional feature such as protein content, protein fortification and amino acid content to a food ingredient or food product. The nutritional feature provided by the rOVA mixture in the composition may be comparable or substantially similar to that of a rOVA containing composition that does not comprise any clipped rOVA. The nutritional feature provided by the rOVA mixture in the composition provided herein may be different from that provided by an rOVA containing composition that does not comprise any clipped rOVA.

The rOVA-containing compositions disclosed herein can provide foaming and foam capacity to a composition. For example, the rOVA mixture can be used for forming a foam to use in baked products, such as cakes, for meringues and other foods where the rOVA mixture can replace egg white to provide foam capacity. In some cases, the rOVA mixture provides foaming and foam capacity of egg-white in absence of any other egg-white proteins.

In some embodiments, the composition provided herein comprising the rOVA mixture (e.g., the rOVA mixture comprising one or more clipped forms of the rOVA) may have a foam height greater than a foam height of an egg white or a composition comprising nOVA. In some cases, the composition provided herein comprising the rOVA mixture (e.g., the rOVA mixture comprising one or more clipped forms of the rOVA) may have a foam height of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. In some cases, the composition provided herein comprising the rOVA mixture (e.g., the rOVA mixture comprising one or more clipped forms of the rOVA) may have a foam height of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. Substitute egg whites may include products such as aquafaba, chia seeds, flax seeds, starches; apple sauce, banana puree; condensed milk, etc. which are commonly used as egg white substitutes.

In some embodiments, the composition provided herein comprising the rOVA mixture (e.g., the rOVA mixture comprising one or more clipped forms of the rOVA) may have a foam height greater than a foam height of rOVA containing composition that does not comprise any clipped rOVA. In some cases, the composition provided herein comprising the rOVA mixture (e.g., the rOVA mixture comprising one or more clipped forms of the rOVA) may have a foam height of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to rOVA containing composition that does not comprise any clipped rOVA. In some cases, the composition provided herein comprising the rOVA mixture (e.g., the rOVA mixture comprising one or more clipped forms of the rOVA) may have a foam height of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to the corresponding rOVA containing composition that does not comprise any clipped rOVA. Substitute egg whites may include products such as aquafaba, chia seeds, flax seeds, starches; apple sauce, banana puree; condensed milk, etc. which are commonly used as egg white substitutes.

In some embodiments, the composition provided herein comprising the rOVA mixture (e.g., the rOVA mixture comprising one or more clipped forms of the rOVA) may have a foam stability greater than a foam stability of an egg white, nOVA compositions or a substitute egg white. In some cases, the composition provided herein comprising the rOVA mixture (e.g., the rOVA mixture comprising one or more clipped forms of the rOVA) may have a foam stability of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white or a substitute egg white. In some cases, the composition provided herein comprising the rOVA mixture (e.g., the rOVA mixture comprising one or more clipped forms of the rOVA) may have a foam stability of up to 50%, 55%, 60%, 65%, 70%, 75%, 80%, 85%, 90%, 95% 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white. In some embodiments, foam stability may be calculated by measuring drainage of a foamed solution. The drainage may be measured in 10-minute increments for 30 minutes to gather data for foam stability. The drained volume after 30 minutes may be compared to the initial liquid volume (5 mL) for instance, foam Stability (%): (Initial volume−drained volume)/initial volume*100.

In some embodiments, the composition provided herein comprising the rOVA mixture (e.g., the rOVA mixture comprising one or more clipped forms of the rOVA) may have a foam stability greater than a foam stability of the corresponding rOVA containing composition that does not comprise any clipped rOVA. In some cases, the composition provided herein comprising the rOVA mixture (e.g., the rOVA mixture comprising one or more clipped forms of the rOVA) may have a foam stability of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to the corresponding rOVA containing composition that does not comprise any clipped rOVA. In some cases, the composition provided herein comprising the rOVA mixture (e.g., the rOVA mixture comprising one or more clipped forms of the rOVA) may have a foam stability of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to the corresponding rOVA containing composition that does not comprise any clipped rOVA. In some embodiments, foam stability may be calculated by measuring drainage of a foamed solution. The drainage may be measured in 10-minute increments for 30 minutes to gather data for foam stability. The drained volume after 30 minutes may be compared to the initial liquid volume (5 mL) for instance, foam Stability (%): (Initial volume−drained volume)/initial volume*100.

In some embodiments, the composition disclosed herein comprising the rOVA mixture (e.g., the rOVA mixture comprising one or more clipped forms of the rOVA) may have a foam capacity greater than a foam capacity of an egg white, nOVA compositions or a substitute egg white. In some cases, a composition comprising rOVA may have a foam capacity of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA, or a substitute egg white. In some cases, a composition comprising rOVA may have a foam capacity of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. Foam capacity may be determined by measuring the initial volume of foam following the whipping and compare against the initial volume of 5 mL. Foam Capacity (%)=(volume of foam/initial volume)*100.

In some embodiments, the composition disclosed herein comprising the rOVA mixture (e.g., the rOVA mixture comprising one or more clipped forms of the rOVA) may have a foam capacity greater than a foam capacity of the corresponding rOVA containing composition that does not comprise any clipped rOVA. In some cases, a composition comprising rOVA may have a foam capacity of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to the corresponding rOVA containing composition that does not comprise any clipped rOVA. In some cases, a composition comprising rOVA may have a foam capacity of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to the corresponding rOVA containing composition that does not comprise any clipped rOVA. Foam capacity may be determined by measuring the initial volume of foam following the whipping and compare against the initial volume of 5 mL. Foam Capacity (%)=(volume of foam/initial volume)*100.

In some embodiments, the liquid composition disclosed herein comprising the rOVA mixture may foam faster than a composition comprising egg whites, nOVA, or a substitute egg white. In some cases, an rOVA composition foams at least 1%, 5%, 10%, 15%, 20%, 25%, 30%, 35%, 40%, 45%, 50%, 55%, 60%, 65%, 70%, 75%, 80%, 85%, 90%, 95%, 100%, faster than an egg white, nOVA, or substitute egg-white composition. In some cases, an rOVA composition foams up to 1%, 5%, 10%, 15%, 20%, 25%, 30%, 35%, 40%, 45%, 50%, 55%, 60%, 65%, 70%, 75%, 80%, 85%, 90%, 95%, 100% faster than an egg white, nOVA, or substitute egg-white composition.

In some embodiments, the liquid composition disclosed herein comprising the rOVA mixture may foam faster than the corresponding rOVA containing composition that does not comprise any clipped rOVA. In some cases, the rOVA mixture containing composition foams at least 1%, 5%, 10%, 15%, 20%, 25%, 30%, 35%, 40%, 45%, 50%, 55%, 60%, 65%, 70%, 75%, 80%, 85%, 90%, 95%, 100%, faster than the corresponding rOVA containing composition that does not comprise any clipped rOVA. In some cases, the rOVA mixture containing composition foams up to 1%, 5%, 10%, 15%, 20%, 25%, 30%, 35%, 40%, 45%, 50%, 55%, 60%, 65%, 70%, 75%, 80%, 85%, 90%, 95%, 100% faster than the corresponding rOVA containing composition that does not comprise any clipped rOVA.

In some embodiments, the liquid composition disclosed herein comprising the rOVA mixture may form a homogenous solution of the rOVA at a higher concentration than the corresponding rOVA containing composition that does not comprise any clipped rOVA. In some cases, the rOVA mixture has higher solubility than the rOVA in a composition that does not comprise any clipped rOVA.

In some embodiments, the composition disclosed herein comprising the rOVA mixture (e.g., the rOVA mixture comprising one or more clipped forms of the rOVA) may have a gel strength greater than a gel strength of an egg white, nOVA composition or an egg white substitutes. In some cases, the rOVA mixture containing composition may have a gel strength within the range from 100 g to 1500 g, from 500 g to 1500 g, or from 700 g to 1500 g. In some cases, the rOVA mixture containing composition has a gel strength of about or at least 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, the rOVA mixture containing composition has a gel strength of up to 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, the rOVA mixture containing composition has a gel strength of about or at least 50%, 55%, 60%, 65%, 70%, 75%, 80%, 85%, 90%, 95%, 100% relative to an egg white, nOVA, or egg white substitutes. In some cases, an rOVA composition has a gel strength of up to 50%, 55%, 60%, 65%, 70%, 75%, 80%, 85%, 90%, 95%, 100% relative to an egg white, nOVA, or egg white substitutes.

In some embodiments, the composition disclosed herein comprising the rOVA mixture (e.g., the rOVA mixture comprising one or more clipped forms of the rOVA) may have a reduced gel strength than the gel strength of the corresponding rOVA containing composition that does not comprise any clipped rOVA. In some cases, the rOVA mixture containing composition may have a gel strength within the range from 100 g to 1500 g, from 500 g to 1500 g, or from 700 g to 1500 g. In some cases, the rOVA mixture containing composition has a gel strength of about or less than 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, the rOVA mixture containing composition has a gel strength of up to 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, the rOVA mixture containing composition has a gel strength of about or less than 10%, 20%, 30%, 40%, 50%, 55%, 60%, 65%, 70%, 75%, 80%, 85%, 90%, 95%, 100% relative to the corresponding rOVA containing composition that does not comprise any clipped rOVA. In some cases, the rOVA mixture containing composition has a gel strength of up to 5%, 10%, 15%, 20%, 25%, 30%, 35%, 40%, 45%, or 50% relative to the corresponding rOVA containing composition that does not comprise any clipped rOVA. In some cases, the rOVA mixture containing composition has no gel strength. In some cases, the rOVA mixture containing composition provides not gelation.

In some embodiments, the composition provided herein comprising the rOVA mixture (e.g., the rOVA mixture comprising one or more clipped forms of the rOVA) can provide structure, texture or a combination of structure and texture. In some embodiments, the rOVA mixture is added to a food ingredient or food product for baking and the rOVA mixture provides structure, texture or a combination of structure and texture to the baked product. In some embodiments, the rOVA mixture can be used in such baked products in place of native egg white, native egg or native egg protein. The addition of the rOVA mixture to baked products can also provide protein fortification to improve the nutritional content. In some embodiments, the rOVA mixture is used in a baked product in an amount between 0.1% and 25% on a weight/weight or weight/volume basis. In some embodiments, the rOVA mixture is used in a baked product in an amount between 0.1% and 5%. In some cases, the rOVA mixture provides the structure and/or texture of egg-white in absence of any other egg-white proteins.

In some embodiments, the composition provided herein comprising the rOVA mixture (e.g., the rOVA mixture comprising one or more clipped forms of the rOVA) can be compatible with gluten formation, such that the rOVA mixture can be used where gluten formation provides structure, texture and/or form to a food ingredient or food product.

Illustrative baked products in which the rOVA mixture can be used as an ingredient include, but are not limited to cake, cookie, bread, bagel, biscuits, muffin, cupcake, scone, pancake, macaroon, choux pastry, meringue, and souffle. For example, the rOVA mixture can be used as an ingredient to make cakes such as pound cake, sponge cake, yellow cake, or angel food cake, where such cakes do not contain any native egg white, native whole egg or native egg protein. Along with the rOVA mixture, baked products may contain additional ingredients such as flour, sweetening agents, gum, hydrocolloids, starches, fibers, flavorings (such as flavoring extracts) and other protein sources. In some embodiments, a baked product may comprise the rOVA mixture and at least one fat or oil, at least one grain starch, and optionally at least one sweetener. Grain starch for use in such compositions include flours such as wheat flour, rice flour, corn flour, millet flour, spelt flour, and oat flour, and starches such as from corn, potato, sorghum, and arrowroot. Oil and fat for use in such compositions include plant-derived oils and fats, such as olive oil, corn oil, avocado oil, nut oils (e.g., almond, walnut and peanut) and safflower oil. R In some embodiments, the OVA mixture may provide such baked goods with at least one characteristic of an egg white such as binding, springiness, aeration, browning, texturizing, humectant, and cohesiveness of the baked product. In some cases, the baked product does not comprise any natural egg white or natural egg, and/or does not include any other egg white derived or egg white related proteins except full length or clipped forms of rOVA. In some cases, the rOVA mixture is provided to the baked composition as an ingredient, such as starting with a concentrate, isolate or powder form of the rOVA mixture. In some cases, the rOVA mixture provided as an ingredient for baked products is at a pH range between about 3.5 and 7.0. In some cases, a sweetener is included in the baked product such as a sugar, plant-derived syrup, honey or sugar-substitute, e.g., an artificial sweetener.

In some embodiments, the compositions provided herein comprising the rOVA mixture (e.g., the rOVA mixture comprising one or more clipped forms of the rOVA) can also be used to prepare egg-less food products, such as food products made where native whole egg or native egg white is a primary or featured ingredient such as scramble, omelet, patty, souffle, quiche, and frittata. In some embodiments, the rOVA mixture provides one or more functional features to the preparation including foaming, coagulation, binding, structure, texture, film-formation, nutritional profile, absence of cholesterol (i.e., cholesterol free), and protein fortification. Such egg-less preparations can be vegan, vegetarian, halal, or kosher, or a combination thereof. An egg-less preparation (also referred to as an egg-white substitute) may comprise the rOVA mixture and at least one fat or oil, a polysaccharide or polysaccharide-containing ingredient, and a starch. In some cases, the egg-less preparation may also include a flavoring agent (such as to provide a salty, sulfur-like, or umami flavor), and/or a coloring agent (for example to provide yellow-like or off-white color to the baked product). In some cases, the inclusion of the rOVA mixture in the egg-less preparation provides a characteristic of natural (native) egg white such as hardness, adhesiveness, fracturability, cohesiveness, gumminess, and chewiness when the composition is heated or cooked. Illustrative polysaccharide or polysaccharide-containing ingredients for such compositions include but not limited to gellan gum, sodium alginate, and psyllium. Oil and fat for use in such compositions include plant-derived oils and fats, such as olive oil, corn oil, avocado oil, and safflower oil.

In some embodiments, the compositions provided herein comprising the rOVA mixture (e.g., the rOVA mixture comprising one or more clipped forms of the rOVA) can be used for a processed meat product or meat-like product, or for fish-like or shell-fish-like products. In such products, the rOVA mixture can provide one or more functional characteristics such as protein content and protein supplementations as well as binding, and texturizing properties. Illustrative meat and meat-like products include burger, patty, sausage, hot dog, sliced deli meat, jerky, bacon, nugget and ground meat-like mixtures. Meat-like products can resemble beef, pork, chicken, lamb, and other edible and consumed meats for humans and for other animals. Fish-like and shell-fish like products can resemble, for example, fish cakes, crab cakes, shrimp, shrimp balls, fish sticks, seafood meat, crab meat, fish fillets and clam strips. In some embodiments, the rOVA mixture is present in an amount between about 0.1% and 30% w/w/or w/v in the meat or meat-like product. In some embodiments, the rOVA mixture is used for a meat-like product (also referred to as a meat-analog and includes at least one fat or oil; and a plant-derived protein. Oil and fat for use in such compositions include plant-derived oils and fats, such as olive oil, corn oil, avocado oil, and safflower oil. Plant-derived proteins for use in meat analogs include soy protein, nut proteins, pea protein, lentil and other pulse proteins and whey protein. In some cases, such plant protein is extruded, in other cases, such plant protein is non-extruded protein. In some cases, a meat analog includes the rOVA mixture at about 2% to 15% (w/w). In some cases, for meat analog compositions, the rOVA mixture acts as a binding agent, a gelling agent or a combination of a binding and gelling agent for such compositions.

In some embodiments, the compositions provided herein comprising the rOVA mixture (e.g., the rOVA mixture comprising one or more clipped forms of the rOVA) can be employed in coatings for food products. For example, the rOVA mixture can provide binding or adhesion characteristics to adhere batter or breading to another food ingredient. In certain embodiments, the rOVA mixture can be used as an “egg-less egg wash” where the rOVA protein (e.g., full length or clipped forms) provides appearance, color, or texture when coated onto other food ingredients or food products, such as baked products. In one example, the “egg-less egg wash” may be used to coat a baked good such that the baked good adheres to a coating (e.g., seed, salt, spice, and herb). The addition of the rOVA mixture as a coating to a food product can provide a crunchy texture or increase the hardness, for example, of the exterior of a food product such as when the product is cooked, baked or fried.

In some embodiments, the compositions provided herein comprising the rOVA mixture (e.g., the rOVA mixture comprising one or more clipped forms of the rOVA) include sauces and dressings, such as an eggless mayonnaise, commercial mayonnaise substitutes, gravy, sandwich spread, salad dressing or food sauce. In some embodiments, the inclusion of the rOVA mixture in a sauce or dressing, and the like, can provide one or more characteristics such as binding, emulsifying, odor neutrality, and mouthfeel. In some embodiments, the rOVA mixture is present in such sauces and dressing in an amount between 0.1% and 3% or between about 3% and about 5% w/w/ or w/v. In some cases, the amount of the rOVA mixture in a sauce or dressing may be substantially similar to the amount of whole egg, egg-white or nOVA used in a commercially available or commonly used recipe. Illustrative sauces and dressing include mayonnaise, commercial mayonnaise substitutes, alfredo sauce, and hollandaise sauce. In some embodiments, the rOVA-containing sauce or dressing does not contain whole egg, egg white, or any other protein derived from egg or related to a native egg. In some cases, the sauce, dressing or other emulsified product made with the rOVA mixture includes at least one fat or oil and water. Illustrative fats and oils for such compositions include corn oil, safflower oil, nut oils, and avocado oil.

In some embodiments, the compositions provided herein comprising the rOVA mixture (e.g., the rOVA mixture comprising one or more clipped forms of the rOVA) can be used to prepare confectionaries such as eggless, animal-free, vegetarian, and vegan confectionaries. In some embodiments, the rOVA mixture can provide one or more functional features to the confectionary including odor neutrality, flavor, mouthfeel, texture, gelling, cohesiveness, foaming, frothiness, nutritional value, and protein fortification. In some embodiments, the prepared confectionary containing the rOVA mixture does not contain any native egg protein or native egg white. In some embodiments, the rOVA mixture in such confectionaries can provide a firm or chewy texture. In some embodiments, the rOVA mixture is present between about 0.1% and 15% in a confectionary. Illustrative confectionaries include a gummy, a taffy, a divinity candy, meringue, marshmallow, and a nougat. In some embodiments, a confectionary includes rOVA, at least one sweetener and optionally a consumable liquid. Illustrative sweeteners include sugar, honey, sugar-substitutes, and plant-derived syrups. In some cases, the rOVA mixture is provided as an ingredient for making confectionaries at a pH between about 3.5 and about 7. In some cases, the rOVA mixture is present in the confectionary composition at about 2% to about 15% (w/v). In some embodiments, the confectionary is a food product such as a meringue, a whipped dessert, or a whipped topping. In some embodiments, the rOVA mixture in the confectionary provides foaming, whipping, fluffing or aeration to the food product, and/or provides gelation. In some cases, the confectionary is a liquid, such as a foamed drink. In some cases, the liquid may include a consumable alcohol (such as in a sweetened cocktail or after-dinner drink).

In some embodiments, the compositions provided herein comprising the rOVA mixture (e.g., the rOVA mixture comprising one or more clipped forms of the rOVA) can be used in dairy products, dairy-like products or dairy containing products. For example, the rOVA mixture can be used in preparations of beverages such as a smoothie, milkshake, “egg-nog”, and coffee beverage. In some embodiments, the rOVA mixture is added to additional ingredients where at least one ingredient is a dairy ingredient or dairy-derived ingredient (such as milk, cream, whey, and butter). In some embodiments, the rOVA mixture is added to additional ingredients to create a beverage that does not contain any native egg protein, native egg white or native egg. In some embodiments, the rOVA mixture is an ingredient in a beverage that does not contain any animal-derived ingredients, such as one that does not contain any native egg-derived or any dairy-derived ingredients. Examples of such non-dairy derived drinks include nut milks, such as soy milk cashew milk, macadamia milk, or almond milk, oat milk, and coconut milk. In some embodiments, the rOVA mixture can also be used to create beverage additions, such as creamer or “milk” to provide protein, flavor, texture and mouthfeel to a beverage such as a coffee, tea, alcohol-based beverages or cocoa. In some embodiments, the rOVA mixture is present in a beverage ingredient or beverage addition in an amount between about 0.1% and 20% w/w or w/v.

In some embodiments herein, the rOVA mixture can be used to prepare a dairy-like product such as yogurt, cheese, or butter. Dairy products with the rOVA mixture can include other animal-based dairy components or proteins. In some embodiments, dairy products prepared with rOVA do not include any animal-based ingredients.

Preparations of dessert products can be prepared using the rOVA mixture. In some embodiments of the dessert products, the rOVA mixture can provide one or more characteristics such as creamy texture, low fat content, odor neutrality, flavor, mouthfeel, texture, binding, and nutritional value. In some embodiments, the rOVA mixture may be present in an ingredient or set of ingredients that is used to prepare a dessert product. Illustrative dessert products suitable for preparation with the rOVA mixture include a mousse, a cheesecake, a custard, a pudding, a popsicle, and a frozen confectionary (e.g., a sherbet, a sorbet, or an ice cream). In some embodiments, dessert products prepared to include the rOVA are mixture vegan, vegetarian or dairy-free. Dessert products that include rOVA can have an amount of the rOVA mixture that is between about 0.1% and about 10% full length or clipped forms of rOVA w/w or w/v.

In some embodiments, the rOVA mixture can be used to prepare a snack food, such as a protein bar, an energy bar, a nutrition bar or a granola bar. The rOVA mixture can provide characteristics to the snack food including one or more of binding, protein supplementation, flavor neutrality, odor neutrality, coating and mouth feel. In some embodiments, the rOVA mixture is added to a preparation of a snack food in an amount between about 0.1% and 30% w/w or w/v.

In some embodiments, the rOVA mixture can be used for nutritional supplements such as in parenteral nutrition, protein drink supplements, and protein shakes where the rOVA mixture provides a high protein supplement. In some embodiments, the rOVA mixture can be added to such compositions in an amount between about 10% and 30% w/w or w/v.

In some embodiments, the compositions provided herein comprising the rOVA mixture (e.g., the rOVA mixture comprising one or more clipped forms of the rOVA) can be used as an egg-replacer or an egg white-replacer. In some embodiments, the rOVA mixture can be mixed or combined with at least one additional component to form the egg white replacer. In some embodiments, the rOVA mixture can provide one or more characteristics to the egg-replacer or egg white-replacer, such as gelling, foaming, whipping, fluffing, binding, springiness, aeration, creaminess and cohesiveness. In some embodiments, the characteristic is the same or better than a native egg or native egg white provided in the same amount or concentration (w/w or w/v). In some embodiments, the egg-replacer or egg white-replacer, does not contain any egg, egg white, protein extracted or isolated from egg.

The rOVA-containing food ingredient and food products, such as described herein, can contain additional ingredients or components. For example, the compositions provided herein comprising the rOVA mixture can be prepared with an additional component such as one or more of a sweetener, a gum, a flavoring, a thickener, an acidulant and an emulsifier. Other ingredients such as flour, grains, oils and fats, fiber, fruit and vegetables can be combined with the rOVA mixture. Such rOVA compositions comprising the rOVA mixture can be vegan, vegetarian, halal, kosher and animal-free, or a combination thereof. In some embodiments, the rOVA mixture can be a food ingredient or prepared for a food product that is normally animal based or normally contains animal-derived components, such as meat, dairy or eggs.

Compositions comprising the rOVA mixture such as food ingredients and food products can be compatible with one or more steps a of consumables preparation such as heated, baked, grilled, roasted, braised, microwaved, broiled, boiled, steamed, extruded, deep fried, or pan-fried, or processed using ohmic heating, Sous Vide, freezing, chilling, blanching, packaging, canning, bleaching, enriching, drying, pressing, grinding, mixing, par cooking, cooking, proofing, marinating, cutting, slicing, dicing, crushing, shredding, chopping, shaking, coring, spiralizing, rolling, juicing, straining, filtering, kneading, whisking, beating, whipping, grating, stuffing, peeling, smoking, curing, salting, preserving, pickling, fermenting, homogenizing, pasteurizing, sterilizing, irradiating, cold plasma processing, high pressure processing, pulse electric field processing, microwave assisted thermal sterilization, stabilizing, blending, pureeing, fortifying, refining, hydrogenating, aging, extending shelf life, or adding enzymes.

In some embodiments, the composition provided herein is treated with heat. In some embodiments, the composition provided herein is not treated with heat. In some cases, the composition is treated with heat at 45° C. to 70° C. In some cases, the composition is treated at 45° C. to 65° C. In some cases, the composition is treated at 45° C. to 60° C. In some cases, the composition is treated at 50° C. to 70° C. In some case, the composition is treated at 50° C. to 65° C. In some cases, the composition is treated at 50° C. to 60° C. In some cases, the composition is treated at 55° C. to 65° C. In some embodiments, the composition is treated for 1, 2, 3, 4, 5, 6, 7 8, 9, or 10 hours. In some embodiments, the composition is treated for 1-2 hours, 1-3 hours, 1-4 hours, 1-5 hours, 1-6 hours, 1-7 hours, 1-8 hours, 1-9 hours, or 1-10 hours. In one embodiment, the composition is treated for 1-3 hours. In a specific embodiment, the composition is treated at 50° C. to 70° C. for 1-3 hours.

Food ingredients and food products prepared with the rOVA mixture can be essentially free of any microbial cells or microbial cell debris. For instance, rOVA may be secreted from a microbial host cell and isolated from microbial cells, culture media and/or microbial cell debris.

In some embodiments, the rOVA mixture may be prepared as a whole cell extract or fractionated extract such that an rOVA composition contains microbial cells and/or microbial cell components.

In one embodiment, an rOVA composition is prepared for animal consumption where the rOVA mixture is present in a whole cell extract or fractionated extract such that an rOVA composition contains microbial cells and/or microbial cell components. In some embodiments, an rOVA composition is prepared for animal consumption where the rOVA mixture is isolated from microbial cells, culture media and microbial cell debris. Illustrative compositions for animal consumption can include a pet food, an animal feed, a chewy treat, bone broth, smoothie or other liquid for animal nutrition and a solid nutritional supplement suitable for animal consumption. In these cases, the microbial cell extract or microbial cell debris may provide additional nutritional value.

Animals which may consume rOVA compositions can include companion animals (e.g., dog, cat, horse), farm animals, exotic animals (lion, tiger, zebra) as well as livestock (such as cow, pig, sheep, goat). The rOVA compositions comprising the rOVA mixture as described herein can also be used for aquaculture (such as for fish and shell fish) and for avian nutrition (such as for bird pets, zoo birds, wild birds, fowl and birds raised for human and animal food).

In some embodiments of the consumable food compositions described herein, the composition is essentially free of animal-derived components, whey protein, caseinate, fat, lactose, hydrolyzed lactose, soy protein, collagen, hydrolyzed collagen, or gelatin, or any combination thereof. In some embodiments, the composition described herein may be essentially free of cholesterol, glucose, fat, saturated fat, trans fat, or any combination thereof. In some cases, a composition described herein comprises less than 10%, 5%, 4%, 3%, 2%, 1%, or 0.5% fat by dry weight. In some embodiments, the composition may be fat-containing (e.g., such as a mayonnaise and commercial mayonnaise substitutes) and such composition may include up to about 60% fat or a reduced-fat composition (e.g., reduced fat mayonnaise and commercial mayonnaise substitutes) and such composition may include lesser percentages of fat. In some embodiments, the composition that free of an animal-derived component can be considered vegetarian and/or vegan.

In some embodiments, the rOVA powder composition comprises less than 5% ash. The term “ash” is an art-known term and represents inorganics such as one or more ions, elements, minerals, and/or compounds. In some cases, the rOVA powder composition comprises less than 5%, 4.5%, 4%, 3.5%, 3%, 2.5%, 2%, 1.5%1, %, 0.75%, 0.5%, 0.25% or 0.1% ash weight per total weight (w/w) and/or weight per total volume (w/v).

In some embodiments, the moisture content of the rOVA powder composition may be less than 15%. The rOVA powder composition may have less than 15%, 12%, 10%, 8%, 6%, 5%, 3%, 2% or 1% moisture weight per total weight (w/w) and/or weight per total volume (w/v). In some embodiments, the carbohydrate content of the rOVA powder composition may be less than 30%. The rOVA powder composition may have less than 30%, 27%, 25%, 22%, 20%, 17%, 15%, 12%, 10%, 8%, 5%, 3% or 1% carbohydrate content w/w or w/v.

### Sensory Neutrality and Improved Sensory Appeal

In some embodiments, in addition to the egg-white like properties, the addition of the rOVA mixture (e.g., the rOVA mixture comprising one or more clipped forms of the rOVA) to a consumable food composition provides increased protein nutritional content, sensory neutrality or an improved sensory appeal as compared to other proteins in such compositions. As used herein “sensory neutrality” refers to the absence of a strong or distinctive taste, odor (smell) or combination of taste and smell, as well as texture, mouth-feel, aftertaste and color. A sensory panel such as one described in Kemp et al. 2009 may be used by a trained sensory analyst. Sensory neutrality may provide an improved sensory appeal to a taster, such as a tester of foods or a consumer, when a consumable food composition containing the rOVA mixture is compared with another like composition that has a different protein such as nOVA, whey protein, pea protein, soy protein, whole egg or egg white protein at the same concentration.

In some embodiments, the rOVA mixture (e.g., the rOVA mixture comprising one or more clipped forms of the rOVA) when added to a consumable food composition is substantially odorless, such as measured by a trained sensory analyst, in comparison with different solutions/products with a different protein component present in an equal concentration to the rOVA containing solution/product, for example, in the comparison is whey, soy, collagen, pea, egg white solid isolates and/or nOVA. In some embodiments of the rOVA compositions described herein, such compositions are essentially odorless at a protein concentration between about 0.5-1%, 1%-5%, 5-10%, 10-15%, 15-20%, 20-25%, 25-30% rOVA weight per total weight (w/w) and/or weight per total volume (w/v) or at a protein concentration of about 0.1, 1, 5, 10, 11, 12, 13, 14, 15, 16, 17, 18, 19, 20, 21, 22, 23, 24, 25, 26, 27, 28, 29 or 30 g of total rOVA protein per 100 mL solution (e.g., per 100 mL water).

In some embodiments, the addition of the rOVA mixture to a consumable food composition also provides a neutral taste in addition to the characteristics such as egg-white like properties and increased protein nutrition content. In some embodiments, the neutral taste can be measured for example, by a trained sensory analyst in comparison with solutions containing a different protein present in an equal concentration to the rOVA, for example, whey, soy, collagen, pea, whole egg, and egg white solid isolates (including native OVA).

In some embodiments, the addition of the rOVA mixture provides a reduction in a certain odor and/or taste that is associated with other proteins or egg-whites. For example, addition of the rOVA mixture has less of an “egg-like” odor or taste as compared to the addition of whole egg, fractionated egg or egg-white to a consumable food composition. In some embodiments, addition of the rOVA mixture has less of a metallic odor or taste as compared to other protein sources.

In some embodiments, the addition of the rOVA mixture has an improved mouth-feel as compared to the addition of other protein sources used to produce egg-white like properties. For example, the addition of the rOVA mixture is less grainy or has less precipitates or solids as compared to other protein sources.

In some embodiments, the addition of the rOVA mixture has an improved texture, for example, as compared to other available supplemental protein sources.

In some embodiments, the consumable composition has a hardness different for a hardness of a control consumable composition, wherein the control consumable composition is substantially identical to the consumable composition except the control consumable composition comprises only rOVA or native ovalbumin (nOVA) as its ovalbumin content. In some embodiments, the hardness of the consumable composition comprising a mixture of rOVA and rcOVA is reduced as compared to the control composition, wherein the control composition comprises only rOVA or native ovalbumin (nOVA) as its ovalbumin content.

In some embodiments, the consumable composition has a chewiness different than a chewiness of a control consumable composition, wherein the control consumable composition is substantially identical to the consumable composition except the control consumable composition comprises only rOVA or native ovalbumin (nOVA) as its ovalbumin content. In some embodiments, the chewiness of the consumable composition comprising a mixture of rOVA and rcOVA is reduced as compared to the control composition, wherein the control composition comprises only rOVA or native ovalbumin (nOVA) as its ovalbumin content.

In some embodiments, the consumable composition has a texture different from a texture of a control consumable composition, wherein the control consumable composition is substantially identical to the consumable composition except the control consumable composition comprises only rOVA or native ovalbumin (nOVA) as its ovalbumin content. In some embodiments, the texture of the consumable composition comprising a mixture of rOVA and rcOVA is improved as compared to the control composition, wherein the control composition comprises only rOVA or native ovalbumin (nOVA) as its ovalbumin content.

In some embodiments, the consumable composition comprising the rOVA mixture (e.g., the rOVA mixture comprising one or more clipped forms of the rOVA) disclosed herein may also have an improved sensory appeal as compared to the composition without the rOVA mixture (such as a composition comprising only rOVA) or with a different protein present in an equal concentration to the rOVA mixture. Alternatively, a control consumable composition comprising only rOVA as the ovalbumin content may have an improved sensory appeal as compared to a consumable composition comprising a rOVA and rcOVA mixture. Such improved sensory appeal may relate to taste and/or smell. Taste and smell can be measured, for example, by a trained sensory analyst. In some instances, a sensory analyst compares a consumable composition with the rOVA mixture to one without it or with a different protein or protein source in an equivalent amount.

As described herein, the consumable compositions comprising the rOVA mixture (e.g., the rOVA mixture comprising one or more clipped forms of the rOVA) herein can be in a liquid form. In some embodiments, the liquid form can be an intermediate product such as soluble rOVA solution. In some cases, the liquid form can be a final product, such as a beverage comprising the rOVA mixture. Example of different types of beverages contemplated herein include: a juice, a soda, a soft drink, a flavored water, a protein water, a fortified water, a carbonated water, a nutritional drink, an energy drink, a sports drink, a recovery drink, an alcohol-based drink, a heated drink, a coffee-based drink, a tea-based drink, a plant-based milk, a nut milk, a milk based drink, a non-dairy, plant based mild drink, infant formula drink, and a meal replacement drink.

### pH of Compositions

The pH of the composition provided herein comprising the rOVA mixture (e.g., the rOVA mixture comprising one or more clipped forms of the rOVA) may be 3.5 to 8. In some embodiments, the pH of the compositions provided herein comprising the rOVA mixture may be at least 3.5. In some embodiments, the pH of the compositions provided herein comprising the rOVA mixture may be at most 8. In some embodiments, the pH of the compositions provided herein comprising the rOVA mixture may be 3.5 to 4, 3.5 to 4.5, 3.5 to 5, 3.5 to 5.5, 3.5 to 6, 3.5 to 6.5, 3.5 to 7, 3.5 to 7.5, 3.5 to 8, 4 to 4.5, 4 to 5, 4 to 5.5, 4 to 6, 4 to 6.5, 4 to 7, 4 to 7.5, 4 to 8, 4.5 to 5, 4.5 to 5.5, 4.5 to 6, 4.5 to 6.5, 4.5 to 7, 4.5 to 7.5, 4.5 to 8, 5 to 5.5, 5 to 6, 5 to 6.5, 5 to 7, 5 to 7.5, 5 to 8, 5.5 to 6, 5.5 to 6.5, 5.5 to 7, 5.5 to 7.5, 5.5 to 8, 6 to 6.5, 6 to 7, 6 to 7.5, 6 to 8, 6.5 to 7, 6.5 to 7.5, 6.5 to 8, 7 to 7.5, 7 to 8, or 7.5 to 8. In some embodiments, the pH of the compositions provided herein comprising the rOVA mixture may be 3.5, 4, 4.5, 5, 5.5, 6, 6.5, 7, 7.5, or 8. In some embodiments, the compositions provided herein comprising the rOVA mixture with a pH between 3.5 to 7 may have one or more improved functionalities as compared to nOVA, egg white, or egg-white substitute compositions. In some embodiments, the composition provided herein comprising the rOVA mixture with a pH between 3.5 to 7 may have one or more improved functionalities as compared to the corresponding composition that does not comprise clipped forms of the rOVA.

In some embodiments, the pH of the compositions provided herein comprising the rOVA mixture may be 2 to 3.5. In some embodiments, the pH of the compositions provided herein comprising the rOVA mixture may be at least 2. In some embodiments, the pH of the compositions provided herein comprising the rOVA mixture may be at most 3.5. In some embodiments, the pH of the compositions provided herein comprising the rOVA mixture may be 2 to 2.5, 2 to 3, 2 to 3.5, 2.5 to 3, 2.5 to 3.5, or 3 to 3.5. In some embodiments, the pH of the compositions provided herein comprising the rOVA mixture may be 2, 2.5, 3, or 3.5.

In some embodiments, the pH of the compositions provided herein comprising the rOVA mixture may be 7 to 12. In some embodiments, the pH of the compositions provided herein comprising the rOVA mixture may be at least 7 In some embodiments, the pH of the compositions provided herein comprising the rOVA mixture may be at most 12. In some embodiments, the pH of the compositions provided herein comprising the rOVA mixture may be 7 to 7.5, 7 to 8, 7 to 8.5, 7 to 9, 7 to 9.5, 7 to 10, 7 to 10.5, 7 to 11, 7 to 11.5, 7 to 12, 7.5 to 8, 7.5 to 8.5, 7.5 to 9, 7.5 to 9.5, 7.5 to 10, 7.5 to 10.5, 7.5 to 11, 7.5 to 11.5, 7.5 to 12, 8 to 8.5, 8 to 9, 8 to 9.5, 8 to 10, 8 to 10.5, 8 to 11, 8 to 11.5, 8 to 12, 8.5 to 9, 8.5 to 9.5, 8.5 to 10, 8.5 to 10.5, 8.5 to 11, 8.5 to 11.5, 8.5 to 12, 9 to 9.5, 9 to 10, 9 to 10.5, 9 to 11, 9 to 11.5, 9 to 12, 9.5 to 10, 9.5 to 10.5, 9.5 to 11, 9.5 to 11.5, 9.5 to 12, 10 to 10.5, 10 to 11, 10 to 11.5, 10 to 12, 10.5 to 11, 10.5 to 11.5, 10.5 to 12, 11 to 11.5, 11 to 12, or 11.5 to 12. In some embodiments, the pH of the compositions provided herein comprising the rOVA mixture may be 7, 7.5, 8, 8.5, 9, 9.5, 10, 10.5, 11, 11.5, or 12.

In some embodiments, the pH of the rOVA mixture may be adjusted prior to its inclusion in a composition or its use as an ingredient. In some embodiments, the pH of the rOVA mixture is adjusted during the purification and/or isolation processes. In some embodiments, the pH of the rOVA mixture for use in an ingredient or in production of a food product composition is adjusted to between about 3.5 to about 7.0. In some cases, the pH of rOVA mixture may be adjusted to more than one pH during the production process. For example, the rOVA may be expressed in a host cell such as a microbial cell, and in some cases the rOVA mixture is secreted by the host cell into the growth media (e.g., liquid media). In some embodiments, the rOVA mixture is separated from the host cells and such separation step may be performed at a selected pH, for example at a pH of about 3.5. In some cases, the rOVA mixture at such separation pH may not be soluble or may not be fully soluble and the pH is adjusted to a higher pH, such as about pH 12. In some embodiments, the rOVA mixture may then be adjusted to a final pH between about 3.5 and about 7.0. Separation of the rOVA mixture from other components of the host cells or other components of the liquid media can include one or more of ion exchange chromatography, such as cation exchange chromatography and/or anion exchange chromatography, filtration and ammonium sulfate precipitation.

### Additional Components of Compositions

The consumable food compositions containing the rOVA mixture (e.g., the rOVA mixture comprising one or more clipped forms of the rOVA) disclosed herein and the methods of making such compositions may including adding or mixing the rOVA mixture with one or more ingredients. For example, food additives may be added in or mixed with the compositions. In some embodiments, food additives can add volume and/or mass to a composition. In some embodiments, the food additive may improve functional performance and/or physical characteristics. For example, a food additive may prevent gelation or increased viscosity due to the lipid portion of the lipoproteins in the freeze-thaw cycle. In some embodiments, the anticaking agent may be added to make a free-flowing composition. In some embodiments, carbohydrates can be added to increase resistance to heat damage, e.g., less protein denaturation during drying and improve stability and flowability of dried compositions. Food additives include, but are not limited to, food coloring, pH adjuster, natural flavoring, artificial flavoring, flavor enhancer, batch marker, food acid, filler, anticaking agent (e.g., sodium silico aluminate), antigreening agent (e.g., citric acid), food stabilizer, foam stabilizer or binding agent, antioxidant, acidity regulatory, bulking agent, color retention agent, whipping agent (e.g., ester-type whipping agent, triethyl citrate, sodium lauryl sulfate), emulsifier (e.g., lecithin), humectant, thickener, excipient, solid diluent, salts, nutrient, sweetener, glazing agent, preservative, vitamin, dietary elements, carbohydrates, polyol, gums, starches, flour, oil, or bran.

Food coloring includes, but is not limited to, FD&C Yellow #5, FD&C Yellow #6, FD&C Red #40, FD&C Red #3, FD&C Blue No. 1, FD&C Blue No. 2, FD&C Green No. 3, carotenoids (e.g., saffron, β-carotene), anthocyanins, annatto, betanin, butterfly pea, caramel coloring, chlorophyllin, elderberry juice, lycopene, carmine, pandan, paprika, turmeric, curcuminoids, quinoline yellow, carmoisine, Ponceau 4R, Patent Blue V, and Green S.

Ingredients for pH adjustment include, but are not limited to, Tris buffer, potassium phosphate, sodium hydroxide, potassium hydroxide, citric acid, sodium citrate, sodium bicarbonate, and hydrochloric acid.

Salts include, but are not limited, to acid salts, alkali salts, organic salts, inorganic salts, phosphates, chloride salts, sodium salts, sodium chloride, potassium salts, potassium chloride, magnesium salts, magnesium chloride, magnesium perchlorate, calcium salts, calcium chloride, ammonium chloride, iron salts, iron chlorides, zinc salts, and zinc chloride.

Nutrient includes, but is not limited to, macronutrient, micronutrient, essential nutrient, non-essential nutrient, dietary fiber, amino acid, essential fatty acids, omega-3 fatty acids, and conjugated linoleic acid.

Sweeteners include, but are not limited to, sugar substitute, artificial sweetener, acesulfame potassium, advantame, alitame, aspartame, sodium cyclamate, dulcin, glucin, neohesperidin dihydrochalcone, neotame, P-4000, saccharin, aspartame-acesulfame salt, sucralose, brazzein, curculin, glycyrrhizin, glycerol, inulin, mogroside, mabinlin, malto-oligosaccharide, mannitol, miraculin, monatin, monellin, osladin, pentadin, stevia, trilobatin, and thaumatin.

Carbohydrates include, but are not limited to, sugar, sucrose, glucose, fructose, galactose, lactose, maltose, mannose, allulose, tagatose, xylose, arabinose, high fructose corn syrup, high maltose corn syrup, corn syrup (e.g., glucose-free corn syrup), sialic acid, monosaccharides, disaccharides, polysaccharides (e.g., polydextrose, maltodextrin), and starch.

Polyols include, but are not limited to, xylitol, maltitol, erythritol, sorbitol, threitol, arabitol, hydrogenated starch hydrolysates, isomalt, lactitol, mannitol, and galactitol (dulcitol).

Gums include, but are not limited to, gum arabic, gellan gum, guar gum, locust bean gum, acacia gum, cellulose gum, and xanthan gum.

Vitamins include, but are not limited to, niacin, riboflavin, pantothenic acid, thiamine, folic acid, vitamin A, vitamin B6, vitamin B12, vitamin D, vitamin E, lutein, zeaxanthin, choline, inositol, and biotin.

Dietary elements include, but are not limited to, calcium, iron, magnesium, phosphorus, potassium, sodium, zinc, copper, manganese, selenium, chlorine, iodine, sulfur, cobalt, molybdenum, nickel, and bromine.

rOVA Protein in the rOVA Mixture and Production of rOVA Protein and the rOVA Mixture

The rOVA in the rOVA can have an amino acid sequence from any species. F or example, the rOVA can have an amino acid sequence of OVA from a bird or a reptile or other egg-laying species. In some embodiments, the rOVA having an amino acid sequence from an avian can be selected from the group consisting of: poultry, fowl, waterfowl, game bird, chicken, quail, turkey, duck, ostrich, goose, gull, guineafowl, pheasant, emu, and any combination thereof. In some embodiments, the rOVA can have an amino acid sequence derived from a single species, such as Gallus gallus domesticus. In some embodiments, the rOVA can have an amino acid sequence derived from two or more species, and as such can be a hybrid.

Illustrative OVA amino acid sequences contemplated herein are provided in Table 1 below as SEQ ID NOs: 1-75.

TABLE 1
OVA Seauences

| | SEQ | |
|---|---|---|
| Name | ID | Sequence |
| Chicken | 1 | MRFPSIFTAVLFAASSALAAPVNTTTEDETAQIPAEAVIGYSDLEGDFDVAVLP |
| Ovalbumin with | | FSNSTNNGLLFINTTIASIAAKEEGVSLDKREAEAGSIGAASMEFCFDVFKELKV |
| bolded signal | | HHANENIFYCPIAIMSALAMVYLGAKDSTRTQINKVVRFDKLPGFGDSIEAQCGTS |
| sequence | | VNVHSSLRDILNQITKPNDVYSFSLASRLYAEERYPILPEYLQCVKELYRGGLEPIN |
| (Potential | | FQTAADQARELINSWVESQTNGIIRNVLQPSSVDSQTAMVLVNAIVFKGLWEKAF |
| clipping site- | | KDEDTQAMPFRVTEQESKPVQMMYQIGLFRVASMASEKMKILELPFASGTMSML |
| Ala352(P1)- | | VLLPDEVSGLEQLESIINFEKLTEWTSSNVMEERKIKVYLPRMKMEEKYNLTSVL |
| Ser353(P1′) | | MAMGITDVFSSSANLSGISSAESLKISQAVHAAHAEINEAGREVVGSAEAGVDAA |
| italicized) | | SVSEEFRADHPFLFCIKHIATNAVLFFGRCVSP |
| Chicken OVA | 2 | EAEAGSIGAASMEFCFDVFKELKVHHANENIFYCPIAIMSALAMVYLGAKDST |
| sequence as | | RTQINKVVRFDKLPGFGDSIEAQCGTSVNVHSSLRDILNQITKPNDVYSFSLAS |
| secreted from | | RLYAEERYPILPEYLQCVKELYRGGLEPINFQTAADQARELINSWVESQTNGI |
| pichia | | IRNVLQPSSVDSQTAMVLVNAIVFKGLWEKAFKDEDTQAMPFRVTEQESKPV |
| | | QMMYQIGLFRVASMASEKMKILELPFASGTMSMLVLLPDEVSGLEQLESIIN |
| | | FEKLTEWTSSNVMEERKIKVYLPRMKMEEKYNLTSVLMAMGITDVFSSSAN |
| | | LSGISSAESLKISQAVHAAHAEINEAGREVVGSAEAGVDAASVSEEFRADHPF |
| | | LFCIKHIATNAVLFFGRCVSP |
| Predicted | 3 | MRVPAQLLGLLLLWLPGARCGSIGAASMEFCFDVFKELKVHHANENIFYCPIAIM |
| Ovalbumin | | SALAMVYLGAKDSTRTQINKVVRFDKLPGFGDSIEAQCGTSVNVHSSLRDILNQIT |
| [Achromobacter | | KPNDVYSFSLASRLYAEERYPILPEYLQCVKELYRGGLEPINFQTAADQARELINS |
| denitrificans] | | WVESQTNGIIRNVLQPSSVDSQTAMVLVNAIVFKGLWEKAFKDEDTQAMPFRVT |
| | | EQESKPVQMMYQIGLFRVASMASEKMKILELPFASGTMSMLVLLPDEVSGLEQL |
| | | ESIINFEKLTEWTSSNVMEERKIKVYLPRMKMEEKYNLTSVLMAMGITDVFSSSA |
| | | NLSGISSAESLKISQAVHAAHAEINEAGREVVGSAEAGVDAASVSEEFRADHPFLF |
| | | CIKHIATNAVLFFGRCVSPLEIKRAAAHHHHHH |
| OLLAS epitope- | 4 | MTSGFANELGPRLMGKLTMGSIGAASMEFCFDVFKELKVHHANENIFYCPIAIMS |
| tagged | | ALAMVYLGAKDSTRTQINKVVRFDKLPGFGDSIEAQCGTSVNVHSSLRDILNQIT |
| ovalbumin | | KPNDVYSFSLASRLYAEERYPILPEYLQCVKELYRGGLEPINFQTAADQARELINS |
| | | WVESQTNGIIRNVLQPSSVDSQTAMVLVNAIVFKGLWEKTFKDEDTQAMPFRVT |
| | | EQESKPVQMMYQIGLFRVASMASEKMKILELPFASGTMSMLVLLPDEVSGLEQL |
| | | ESIINFEKLTEWTSSNVMEERKIKVYLPRMKMEEKYNLTSVLMAMGITDVFSSSA |
| | | NLSGISSAESLKISQAVHAAHAEINEAGREVVGSAEAGVDAASVSEEFRADHPFLF |
| | | CIKHIATNAVLFFGRCVSPSR |
| Serpin family | 5 | MGGRRVRWEVYISRAGYVNRQIAWRRHHRSLTMRVPAQLLGLLLLWLPGARCG |
| protein | | SIGAASMEFCFDVFKELKVHHANENIFYCPIAIMSALAMVYLGAKDSTRTQINKV |
| [Achromobacter | | VRFDKLPGFGDSIEAQCGTSVNVHSSLRDILNQITKPNDVYSFSLASRLYAEERYPI |
| denitrificans] | | LPEYLQCVKELYRGGLEPINFQTAADQARELINSWVESQTNGIIRNVLQPSSVDSQ |
| | | TAMVLVNAIVFKGLWEKAFKDEDTQAMPFRVTEQESKPVQMMYQIGLFRVASM |
| | | ASEKMKILELPFASGTMSMLVLLPDEVSGLEQLESIINFEKLTEWTSSNVMEERKI |
| | | KVYLPRMKMEEKYNLTSVLMAMGITDVFSSSANLSGISSAESLKISQAVHAAHAE |
| | | INEAGREVVGSAEAGVDAASVSEEFRADHPFLFCIKHIATNAVLFFGRCVSPLEIK |
| | | RAAAHHHHHH |
| PREDICTED: | 6 | MGSIGAVSMEFCFDVFKELKVHHANENIFYSPFTIISALAMVYLGAKDSTRTQINK |
| ovalbumin | | VVRFDKLPGFGDSVEAQCGTSVNVHSSLRDILNQITKPNDVYSFSLASRLYAEETY |
| isoform X1 | | PILPEYLQCVKELYRGGLESINFQTAADQARGLINSWVESQTNGMIKNVLQPSSV |
| [Meleagris | | DSQTAMVLVNAIVFKGLWEKAFKDEDTQAIPFRVTEQESKPVQMMYQIGLFKVA |
| gallopavo] | | SMASEKMKILELPFASGTMSMWVLLPDEVSGLEQLETTISFEKMTEWISSNIMEER |
| | | RIKVYLPRMKMEEKYNLTSVLMAMGITDLFSSSANLSGISSAGSLKISQAVHAAY |
| | | AEIYEAGREVIGSAEAGADATSVSEEFRVDHPFLYCIKHNLTNSILFFGRCISP |
| Ovalbumin | 7 | MGSIGAVSMEFCFDVFKELKVHHANENIFYSPFTIISALAMVYLGAKDSTRTQINK |
| precursor | | VVRFDKLPGFGDSVEAQCGTSVNVHSSLRDILNQITKPNDVYSFSLASRLYAEETY |
| [Meleagris | | PILPEYLQCVKELYRGGLESINFQTAADQARGLINSWVESQTNGMIKNVLQPSSV |
| gallopavo] | | DSQTAMVLVNAIVFKGLWEKAFKDEDTQAIPFRVTEQESKPVQMMYQIGLFKVA |
| | | SMASEKMKILELPFASGTMSMWVLLPDEVSGLEQLETTISFEKMTEWISSNIMEER |
| | | RIKVYLPRMKMEEKYNLTSVLMAMGITDLFSSSANLSGISSAGSLKISQAAHAAY |
| | | AEIYEAGREVIGSAEAGADATSVSEEFRVDHPFLYCIKHNLTNSILFFGRCISP |
| Hypothetical | 8 | YYRVPCMVLCTAFHPYIFIVLLFALDNSEFTMGSIGAVSMEFCFDVFKELRVHHPN |
| protein | | ENIFFCPFAIMSAMAMVYLGAKDSTRTQINKVIRFDKLPGFGDSTEAQCGKSANV |
| [Bambusicola | | HSSLKDILNQITKPNDVYSFSLASRLYADETYSIQSEYLQCVNELYRGGLESINFQT |
| thoracicus] | | AADQARELINSWVESQTNGIIRNVLQPSSVDSQTAMVLVNAIVFRGLWEKAFKDE |
| | | DTQTMPFRVTEQESKPVQMMYQIGSFKVASMASEKMKILELPLASGTMSMLVLL |
| | | PDEVSGLEQLETTISFEKLTEWTSSNVMEERKIKVYLPRMKMEEKYNLTSVLMA |
| | | MGITDLFRSSANLSGISLAGNLKISQAVHAAHAEINEAGRKAVSSAEAGVDATSV |
| | | SEEFRADRPFLFCIKHIATKVVFFFGRYTSP |
| Egg albumin | 9 | MGSIGAASMEFCFDVFKELKVHHANDNMLYSPFAILSTLAMVFLGAKDSTRTQIN |
| | | KVVHFDKLPGFGDSIEAQCGTSVNVHSSLRDILNQITKQNDAYSFSLASRLYAQET |
| | | YTVVPEYLQCVKELYRGGLESVNFQTAADQARGLINAWVESQTNGIIRNILQPSS |
| | | VDSQTAMVLVNAIAFKGLWEKAFKAEDTQTIPFRVTEQESKPVQMMYQIGSFKV |
| | | ASMASEKMKILELPFASGTMSMLVLLPDDVSGLEQLESIISFEKLTEWTSSSIMEER |
| | | KVKVYLPRMKMEEKYNLTSLLMAMGITDLFSSSANLSGISSVGSLKISQAVHAAH |
| | | AEINEAGRDVVGSAEAGVDATEEFRADHPFLFCVKHIETNAILLFGRCVSP |
| Ovalbumin | 10 | MASIGAVSTEFCVDVYKELRVHHANENIFYSPFTIISTLAMVYLGAKDSTRTQINK |
| isoform X2 | | VVRFDKLPGFGDSIEAQCGTSVNVHSSLRDILNQITKPNDVYSFSLASRLYAEETY |
| [Numida | | PILPEYLQCVKELYRGGLESINFQTAADQARELINSWVESQTSGIIKNVLQPSSVNS |
| meleagris] | | QTAMVLVNAIYFKGLWERAFKDEDTQAIPFRVTEQESKPVQMMSQIGSFKVASV |
| | | ASEKVKILELPFVSGTMSMLVLLPDEVSGLEQLESTISTEKLTEWTSSSIMEERKIK |
| | | VFLPRMRMEEKYNLTSVLMAMGMTDLFSSSANLSGISSAESLKISQAVHAAYAEI |
| | | YEAGREVVSSAEAGVDATSVSEEFRVDHPFLLCIKHNPTNSILFFGRCISP |
| Ovalbumin | 11 | MALCKAFHPYIFIVLLFDVDNSAFTMASIGAVSTEFCVDVYKELRVHHANENIFYS |
| isoform X1 | | PFTIISTLAMVYLGAKDSTRTQINKVVRFDKLPGFGDSIEAQCGTSVNVHSSLRDIL |
| [Numida | | NQITKPNDVYSFSLASRLYAEETYPILPEYLQCVKELYRGGLESINFQTAADQARE |
| meleagris] | | LINSWVESQTSGIIKNVLQPSSVNSQTAMVLVNAIYFKGLWERAFKDEDTQAIPFR |
| | | VTEQESKPVQMMSQIGSFKVASVASEKVKILELPFVSGTMSMLVLLPDEVSGLEQ |
| | | LESTISTEKLTEWTSSSIMEERKIKVFLPRMRMEEKYNLTSVLMAMGMTDLFSSSA |
| | | NLSGISSAESLKISQAVHAAYAEIYEAGREVVSSAEAGVDATSVSEEFRVDHPFLL |
| | | CIKHNPTNSILFFGRCISP |
| PREDICTED: | 12 | MGSIGAASMEFCFDVFKELKVHHANDNMLYSPFAILSTLAMVFLGAKDSTRTQIN |
| Ovalbumin | | KVVHFDKLPGFGDSIEAQCGTSANVHSSLRDILNQITKQNDAYSFSLASRLYAQET |
| isoform X2 | | YTVVPEYLQCVKELYRGGLESVNFQTAADQARGLINAWVESQTNGIIRNILQPSS |
| [Coturnix | | VDSQTAMVLVNAIAFKGLWEKAFKAEDTQTIPFRVTEQESKPVQMMHQIGSFKV |
| japonica] | | ASMASEKMKILELPFASGTMSMLVLLPDDVSGLEQLESTISFEKLTEWTSSSIMEE |
| | | RKVKVYLPRMKMEEKYNLTSLLMAMGITDLFSSSANLSGISSVGSLKISQAVHAA |
| | | YAEINEAGRDVVGSAEAGVDATEEFRADHPFLFCVKHIETNAILLFGRCVSP |
| PREDICTED: | 13 | MGLCTAFHPYIFIVLLFALDNSEFTMGSIGAASMEFCFDVFKELKVHHANDNMLY |
| ovalbumin | | SPFAILSTLAMVFLGAKDSTRTQINKVVHFDKLPGFGDSIEAQCGTSANVHSSLRD |
| isoform X1 | | ILNQITKQNDAYSFSLASRLYAQETYTVVPEYLQCVKELYRGGLESVNFQTAADQ |
| [Coturnix | | ARGLINAWVESQTNGIIRNILQPSSVDSQTAMVLVNAIAFKGLWEKAFKAEDTQTI |
| japonica] | | PFRVTEQESKPVQMMHQIGSFKVASMASEKMKILELPFASGTMSMLVLLPDDVS |
| | | GLEQLESTISFEKLTEWTSSSIMEERKVKVYLPRMKMEEKYNLTSLLMAMGITDL |
| | | FSSSANLSGISSVGSLKISQAVHAAYAEINEAGRDVVGSAEAGVDATEEFRADHPF |
| | | LFCVKHIETNAILLFGRCVSP |
| Egg albumin | 14 | MGSIGAASMEFCFDVFKELKVHHANDNMLYSPFAILSTLAMVFLGAKDSTRTQIN |
| | | KVVHFDKLPGFGDSIEAQCGTSANVHSSLRDILNQITKQNDAYSFSLASRLYAQET |
| | | YTVVPEYLQCVKELYRGGLESVNFQTAADQARGLINAWVESQTNGIIRNILQPSS |
| | | VDSQTAMVLVNAIAFKGLWEKAFKAEDTQTIPFRVTEQESKPVQMMHQIGSFKV |
| | | ASMASEKMKILELPFASGTMSMLVLLPDDVSGLEQLESTISFEKLTEWTSSSIMEE |
| | | RKVKVYLPRMKMEEKYNLTSLLMAMGITDLFSSSANLSGISSVGSLKIPQAVHAA |
| | | YAEINEAGRDVVGSAEAGVDATEEFRADHPFLFCVKHIETNAILLFGRCVSP |
| ovalbumin [Anas | 15 | MGSIGAASTEFCFDVFRELRVQHVNENIFYSPFSIISALAMVYLGARDNTRTQIDK |
| platyrhynchos] | | VVHFDKLPGFGESMEAQCGTSVSVHSSLRDILTQITKPSDNFSLSFASRLYAEETY |
| | | AILPEYLQCVKELYKGGLESISFQTAADQARELINSWVESQTNGIIKNILQPSSVDS |
| | | QTTMVLVNAIYFKGMWEKAFKDEDTQAMPFRMTEQESKPVQMMYQVGSFKVA |
| | | MVTSEKMKILELPFASGMMSMFVLLPDEVSGLEQLESTISFEKLTEWTSSTMMEE |
| | | RRMKVYLPRMKMEEKYNLTSVFMALGMTDLFSSSANMSGISSTVSLKMSEAVH |
| | | AACVEIFEAGRDVVGSAEAGMDVTSVSEEFRADHPFLFFIKHNPTNSILFFGRWM |
| | | SP |
| PREDICTED: | 16 | MGSIGAASTEFCFDVFRELKVQHVNENIFYSPLSIISALAMVYLGARDNTRTQIDQ |
| ovalbumin-like | | VVHFDKIPGFGESMEAQCGTSVSVHSSLRDILTEITKPSDNFSLSFASRLYAEETYT |
| [Anser cygnoides | | ILPEYLQCVKELYKGGLESISFQTAADQARELINSWVESQTNGIIKNILQPSSVDSQ |
| domesticus] | | TTMVLVNAIYFKGMWEKAFKDEDTQTMPFRMTEQESKPVQMMYQVGSFKLAT |
| | | VTSEKVKILELPFASGMMSMCVLLPDEVSGLEQLETTISFEKLTEWTSSTMMEER |
| | | RMKVYLPRMKMEEKYNLTSVFMALGMTDLFSSSANMSGISSTVSLKMSEAVHA |
| | | ACVEIFEAGRDVVGSAEAGMDVTSVSEEFRADHPFLFFIKHNPSNSILFFGRWISP |
| PREDICTED: | 17 | MGSIGAASTEFCFDVFKELKVQHVNENIFYSPLTIISALSMVYLGARENTRAQIDK |
| Ovalbumin-like | | VLHFDKMPGFGDTIESQCGTSVSIHTSLKDMFTQITKPSDNYSLSFASRLYAEETY |
| [Aquila | | PILPEYLQCVKELYKGGLETISFQTAAEQARELINSWVESQTNGMIKNILQPSSVDP |
| chrysaetos | | QTKMVLVNAIYFKGVWEKAFKDEDTQEVPFRVTEQESKPVQMMYQIGSFKVAV |
| canadensis] | | MASEKMKILELPYASGQLSMLVLLPDDVSGLEQLESAITFEKLMAWTSSTTMEER |
| | | KMKVYLPRMKIEEKYNLTSVLMALGVTDLFSSSANLSGISSAESLKISKAVHEAF |
| | | VEIYEAGSEVVGSTEAGMEVTSVSEEFRADHPFLFLIKHNPTNSILFFGRCFSP |
| PREDICTED: | 18 | MGSIGAASTEFCFDVFKELKVQHVNENIFYSPLTIISALSMVYLGARENTRTQIDK |
| Ovalbumin-like | | VLHFDKMTGFGDTVESQCGTSVSIHTSLKDIFTQITKPSDNYSLSLASRLYAEETYP |
| [Haliaeetus | | ILPEYLQCVKELYKGGLETVSFQTAAEQARELINSWVESQTNGMIKNILQPSSVDP |
| albicilla] | | QTKMVLVNAIYFKGVWEKAFKDEDTQEVPFRVTEQESKPVQMMYQIGSFKVAV |
| | | MASEKMKILELPYASGQLSMLVLLPDDVSGLEQLESAITSEKLMEWTSSTTMEER |
| | | KMKVYLPRMKIEEKYNLTSVLMALGVTDLFSSSADLSGISSAESLKISKAVHEAF |
| | | VEIYEAGSEVVGSTEGGMEVTSVSEEFRADHPFLFLIKHKPTNSILFFGRCFSP |
| PREDICTED: | 19 | MGSIGAASTEFCFDVFKELKVQHVNENIFYSPLTIISALSMVYLGARENTRTQIDK |
| Ovalbumin-like | | VLHFDKMTGFGDTVESQCGTSVSIHTSLKDIFTQITKPSDNYSLSLASRLYAEETYP |
| [Haliaeetus | | ILPEYLQCVKELYKGGLETVSFQTAAEQARELINSWVESQTNGMIKNILQPSSVDP |
| leucocephalus] | | QTKMVLVNAIYFKGVWEKAFKDEDTQEVPFRVTEQESKPVQMMYQIGSFKVAV |
| | | MASEKMKILELPYASGQLSMLVLLPDDVSGLEQLESAITSEKLMEWTSSTTMEER |
| | | KMKVYLPRMKIEEKYNLTSVLMALGVTDLFSSSADLSGISSAESLKISKAVHEAF |
| | | VEIYEAGSEVVGSTEGGMEVTSFSEEFRADHPFLFLIKHKPTNSILFFGRCFSP |
| PREDICTED: | 20 | MGSIGAASTEFCFDVFKELKVQHVNENIFYSPLSIISALSMVYLGARENTRAQIDK |
| Ovalbumin | | VVHFDKITGFGETIESQCGTSVSVHTSLKDMFTQITKPSDNYSLSFASRLYAEETYP |
| [Fulmarus | | ILPEYLQCVKELYKGGLETTSFQTAADQARELINSWVESQTNGMIKNILQPGSVDP |
| glacialis] | | QTEMVLVNAIYFKGMWEKAFKDEDTQAVPFRMTEQESKTVQMMYQIGSFKVAV |
| | | MASEKMKILELPYASGELSMLVMLPDDVSGLEQLETAITFEKLMEWTSSNMMEE |
| | | RKMKVYLPRMKMEEKYNLTSVLMALGVTDLFSSSANLSGISSAESLKMSEAVHE |
| | | AFVEIYEAGSEVVGSTGAGMEVTSVSEEFRADHPFLFLIKHNPTNSILFFGRCFSP |
| PREDICTED: | 21 | MGSIGAASTEFCFDVFKELRVQHVNENVCYSPLIIISALSLVYLGARENTRAQIDK |
| Ovalbumin-like | | VVHFDKITGFGESIESQCGTSVSVHTSLKDMFNQITKPSDNYSLSVASRLYAEERY |
| [Chlamydotis | | PILPEYLQCVKELYKGGLESISFQTAADQAREAINSWVESQTNGMIKNILQPSSVD |
| macqueenii] | | PQTEMVLVNAIYFKGMWQKAFKDEDTQAVPFRISEQESKPVQMMYQIGSFKVAV |
| | | MAAEKMKILELPYASGELSMLVLLPDEVSGLEQLENAITVEKLMEWTSSSPMEER |
| | | IMKVYLPRMKIEEKYNLTSVLMALGITDLFSSSANLSGISAEESLKMSEAVHQAFA |
| | | EISEAGSEVVGSSEAGIDATSVSEEFRADHPFLFLIKHNATNSILFFGRCFSP |
| PREDICTED: | 22 | MGSISAASTEFCFDVFKELKVQHVNENIFYSPLSIISALSMVYLGARENTRAQIEKV |
| Ovalbumin like | | VHFDKITGFGESIESQCSTSVSVHTSLKDMFTQITKPSDNYSLSFASRFYAEETYPIL |
| [Nipponia | | PEYLQCVKELYKGGLETINFRTAADQARELINSWVESQTNGMIKNILQPGSVDPQ |
| nippon] | | TDMVLVNAIYFKGMWEKAFKDEDTQALPFRVTEQESKPVQMMYQIGSFKVAVL |
| | | ASEKVKILELPYASGQLSMLVLLPDDVSGLEQLETAITVEKLMEWTSSNNMEERK |
| | | IKVYLPRIKIEEKYNLTSVLMALGITDLFSSSANLSGISSAESLKVSEAIHEAFVEIYE |
| | | AGSEVAGSTEAGIEVTSVSEEFRADHPFLFLIKHNATNSILFFGRCFSP |
| PREDICTED: | 23 | MVSIGAASTEFCFDVFKELKVQHVNENIFYSPLSIISALSMVYLGARENTRAQIDK |
| Ovalbumin-like | | VVHFDKITGFEETIESQCSTSVSVHTSLKDMFTQITKPSDNYSLSFASRLYAEETYPI |
| isoform X2 | | LPEYLQCVKELYKGGLETISFQTAADQARELINSWVESQTDGMIKNILQPGSVDP |
| [Gavia stellata] | | QTEMVLVNAIYFKGMWEKAFKDEDTQAVPFRMTEQESKPVQMMYQIGSFKVAV |
| | | MASEKMKILELPYASGGMSMLVMLPDDVSGLEQLETAITFEKLMEWTSSNMME |
| | | ERKMKVYLPRMKMEEKYNLTSVLMALGMTDLFSSSANLSGISSAESLKMSEAVH |
| | | EAFVEIYEAGSEAVGSTGAGMEVTSVSEEFRADHPFLFLIKHNPTNSILFFGRCFSP |
| PREDICTED: | 24 | MGSIGAASTEFCFDVFKELKVQHVNENIFYSPLSIISALSMVYLGARENTRAQIDK |
| Ovalbumin | | VVHFDKITGFGEPIESQCGISVSVHTSLKDMITQITKPSDNYSLSFASRLYAEETYPI |
| [Pelecanus | | LPEYLQCVKELYKGGLETISFQTAADQARELINSWVENQTNGMIKNILQPGSVDP |
| crispus] | | QTEMVLVNAVYFKGMWEKAFKDEDTQAVPFRMTEQESKPVQMMYQIGSFKVA |
| | | VMASEKIKILELPYASGELSMLVLLPDDVSGLEQLETAITLDKLTEWTSSNAMEER |
| | | KMKVYLPRMKIEKKYNLTSVLIALGMTDLFSSSANESGISSAESLKMSEAIHEAFL |
| | | EIYEAGSEVVGSTEAGMEVTSVSEEFRADHPFLFLIKHNPTNSILFFGRCLSP |
| PREDICTED: | 25 | MGSIGAASTEFCFDVFKELKVQHVNENIFYSPLTIISALSMVYLGARENTRAQIDK |
| Ovalbumin-like | | VVHFDKIPGFGDTTESQCGTSVSVHTSLKDMFTQITKPSDNYSVSFASRLYAEETY |
| [Charadrius | | PILPEFLECVKELYKGGLESISFQTAADQARELINSWVESQTNGMIKNILQPGSVDS |
| vociferus] | | QTEMVLVNAIYFKGMWEKAFKDEDTQTVPFRMTEQETKPVQMMYQIGTFKVAV |
| | | MPSEKMKILELPYASGELCMLVMLPDDVSGLEELESSITVEKLMEWTSSNMMEE |
| | | RKMKVFLPRMKIEEKYNLTSVLMALGMTDLFSSSANLSGISSAEPLKMSEAVHEA |
| | | FIEIYEAGSEVVGSTGAGMEITSVSEEFRADHPFLFLIKHNPTNSILFFGRCVSP |
| PREDICTED: | 26 | MGSIGAVSTEFCFDVFKELKVQHVNENIFYSPLSIISALSMVYLGARENTRAQIDK |
| Ovalbumin-like | | VVHFDKITGSGETIEAQCGTSVSVHTSLKDMFTQITKPSENYSVGFASRLYADETY |
| [Eurypyga | | PIIPEYLQCVKELYKGGLEMISFQTAADQARELINSWVESQTNGMIKNILQPGSVD |
| helias] | | PQTEMILVNAIYFKGVWEKAFKDEDTQAVPFRMTEQESKPVQMMYQFGSFKVA |
| | | AMAAEKMKILELPYASGALSMLVLLPDDVSGLEQLESAITFEKLMEWTSSNMME |
| | | EKKIKVYLPRMKMEEKYNFTSVLMALGMTDLFSSSANESGISSADSLKMSEVVH |
| | | EAFVEIYEAGSEVVGSTGSGMEAASVSEEFRADHPFLFLIKHNPTNSILFFGRCFSP |
| PREDICTED: | 27 | MVSIGAASTEFCFDVFKELKVQHVNENIFYSPLSIISALSMVYLGARENTRAQIDK |
| Ovalbumin-like | | VVHFDKITGFEETIESQVQKKQCSTSVSVHTSLKDMFTQITKPSDNYSLSFASRLY |
| isoform X1 | | AEETYPILPEYLQCVKELYKGGLETISFQTAADQARELINSWVESQTDGMIKNILQ |
| [Gavia stellata] | | PGSVDPQTEMVLVNAIYFKGMWEKAFKDEDTQAVPFRMTEQESKPVQMMYQIG |
| | | SFKVAVMASEKMKILELPYASGGMSMLVMLPDDVSGLEQLETAITFEKLMEWTS |
| | | SNMMEERKMKVYLPRMKMEEKYNLTSVLMALGMTDLFSSSANLSGISSAESLK |
| | | MSEAVHEAFVEIYEAGSEAVGSTGAGMEVTSVSEEFRADHPFLFLIKHNPTNSILF |
| | | FGRCFSP |
| PREDICTED: | 28 | MGSIGAASGEFCFDVFKELKVQHVNENIFYSPLSIISALSMVYLGARENTRAQIDK |
| Ovalbumin-like | | VVHFDKIIGFGESIESQCGTSVSVHTSLKDMFAQITKPSDNYSLSFASRLYAEETFPI |
| [Egretta | | LPEYLQCVKELYKGGLETLSFQTAADQARELINSWVESQTNGMIKDILQPGSVDP |
| garzetta] | | QTEMVLVNAIYFKGVWEKAFKDEDTQTVPFRMTEQESKPVQMMYQIGSFKVAV |
| | | VAAEKIKILELPYASGALSMLVLLPDDVSSLEQLETAITFEKLTEWTSSNIMEERKI |
| | | KVYLPRMKIEEKYNLTSVLMDLGITDLFSSSANLSGISSAESLKVSEAIHEAIVDIY |
| | | EAGSEVVGSSGAGLEGTSVSEEFRADHPFLFLIKHNPTSSILFFGRCFSP |
| PREDICTED: | 29 | MGSIGAASTEFCFDVFKELKVQHVNENIFYSPLSIISALSMVYLGARENTRAQIDK |
| Ovalbumin-like | | VVHFDKITGSGEAIESQCGTSVSVHISLKDMFTQITKPSDNYSLSFASRLYAEETYP |
| [Balearica | | ILPEYLQCVKELYKEGLATISFQTAADQAREFINSWVESQTNGMIKNILQPGSVDP |
| regulorum | | QTQMVLVNAIYFKGVWEKAFKDEDTQAVPFRMTKQESKPVQMMYQIGSFKVAV |
| gibbericeps] | | MASEKMKILELPYASGQLSMLVMLPDDVSGLEQIENAITFEKLMEWTNPNMMEE |
| | | RKMKVYLPRMKMEEKYNLTSVLMALGMTDLFSSSANLSGISSAESLKMSEAVHE |
| | | AFVEIYEAGSEVVGSTGAGIEVTSVSEEFRADHPFLFLIKHNPTNSILFFGRCFSP |
| PREDICTED: | 30 | MGSIGEASTEFCIDVFRELKVQHVNENIFYSPLSIISALSMVYLGARENTRAQIDQV |
| Ovalbumin-like | | VHFDKITGFGDTVESQCGSSLSVHSSLKDIFAQITQPKDNYSLNFASRLYAEETYPI |
| [Nestor | | LPEYLQCVKELYKGGLETISFQTAADQARELINSWVESQTNGMIKNILQPSSVDPQ |
| notabilis] | | TEMVLVNAIYFKGVWEKAFKDEETQAVPFRITEQENRPVQIMYQFGSFKVAVVA |
| | | SEKIKILELPYASGQLSMLVLLPDEVSGLEQLENAITFEKLTEWTSSDIMEEKKIKV |
| | | FLPRMKIEEKYNLTSVLVALGIADLFSSSANLSGISSAESLKMSEAVHEAFVEIYEA |
| | | GSEVVGSSGAGIEAASDSEEFRADHPFLFLIKHKPTNSILFFGRCFSP |
| PREDICTED: | 31 | MGSIGAASTEFCFDIFNELKVQHVNENIFYSPLSIISALSMVYLGARENTKAQIDKV |
| Ovalbumin-like | | VHFDKITGFGESIESQCSTSASVHTSFKDMFTQITKPSDNYSLSFASRLYAEETYPIL |
| [Pygoscelis | | PEYSQCVKELYKGGLESISFQTAADQARELINSWVESQTNGMIKNILQPGSVDPQT |
| adeliae] | | ELVLVNAIYFKGTWEKAFKDKDTQAVPFRVTEQESKPVQMMYQIGSYKVAVIAS |
| | | EKMKILELPYASGELSMLVLLPDDVSGLEQLETAITFEKLMEWTSSNMMEERKV |
| | | KVYLPRMKIEEKYNLTSVLMALGMTDLFSPSANLSGISSAESLKMSEAIHEAFVEI |
| | | YEAGSEVVGSTEAGMEVTSVSEEFRADHPFLFLIKCNLTNSILFFGRCFSP |
| Ovalbumin-like | 32 | MGSISTASTEFCFDVFKELKVQHVNENIFYSPLSIISALSMVYLGARENTRAQIEKV |
| [Athene | | VHFDKITGFGESIESQCGTSVSVHTSLKDMLIQISKPSDNYSLSFASKLYAEETYPIL |
| cunicularia} | | PEYLQCVKELYKGGLESINFQTAADQARQLINSWVESQTNGMIKDILQPSSVDPQ |
| | | TEMVLVNAIYFKGIWEKAFKDEDTQEVPFRITEQESKPVQMMYQIGSFKVAVIAS |
| | | EKIKILELPYASGELSMLIVLPDDVSGLEQLETAITFEKLIEWTSPSIMEERKTKVYL |
| | | PRMKIEEKYNLTSVLMALGMTDLFSPSANLSGISSAESLKMSEAIHEAFVEIYEAG |
| | | SEVVGSAEAGMEATSVSEFRVDHPFLFLIKHNPANIILFFGRCVSP |
| PREDICTED: | 33 | MGSIGAASTEFCFDVFKELKVQHVNENIFYSPLTIISALSLVYLGARENTRAQIDKV |
| Ovalbumin-like | | FHFDKISGFGETTESQCGTSVSVHTSLKEMFTQITKPSDNYSVSFASRLYAEDTYPI |
| [Calidris | | LPEYLQCVKELYKGGLETISFQTAADQAREVINSWVESQTNGMIKNILQPGSVDS |
| pugnax] | | QTEMVLVNAIYFKGMWEKAFKDEDTQTMPFRITEQERKPVQMMYQAGSFKVAV |
| | | MASEKMKILELPYASGEFCMLIMLPDDVSGLEQLENSFSFEKLMEWTTSNMMEE |
| | | RKMKVYIPRMKMEEKYNLTSVLMALGMTDLFSSSANLSGISSAETLKMSEAVHE |
| | | AFMEIYEAGSEVVGSTGSGAEVTGVYEEFRADHPFLFLVKHKPTNSILFFGRCVSP |
| PREDICTED: | 34 | MGSIGAASTEFCFDIFNELKVQHVNENIFYSPLSIISALSMVYLGARENTKAQIDKV |
| Ovalbumin | | VHFDKITGFGETIESQCSTSVSVHTSLKDTFTQITKPSDNYSLSFASRLYAEETYPIL |
| [Aptenodytes | | PEYSQCVKELYKGGLETISFQTAADQARELINSWVESQTNGMIKNILQPGSVDPQT |
| forsteri] | | ELVLVNAIYFKGTWEKAFKDKDTQAVPFRVTEQESKPVQMMYQIGSYKVAVIAS |
| | | EKMKILELPYASRELSMLVLLPDDVSGLEQLETAITFEKLMEWTSSNMMEERKVK |
| | | VYLPRMKIEEKYNLTSVLMALGMTDLFSPSANLSGISSAESLKMSEAVHEAFVEIY |
| | | EAGSEVVGSTGAGMEVTSVSEEFRADHPFLFLIKCNPTNSILFFGRCFSP |
| PREDICTED: | 35 | MGSISAASAEFCLDVFKELKVQHVNENIFYSPLSIISALSMVYLGARENTRAQIDK |
| Ovalbumin-like | | VVHFDKITGSGETIEFQCGTSANIHPSLKDMFTQITRLSDNYSLSFASRLYAEERYP |
| [Pterocles | | ILPEYLQCVKELYKGGLETISFQTAADQARELINSWVESQTNGMIKNILQPGSVNP |
| gutturalis] | | QTEMVLVNAIYFKGLWEKAFKDEDTQTVPFRMTEQESKPVQMMYQVGSFKVAV |
| | | MASDKIKILELPYASGELSMLVLLPDDVTGLEQLETSITFEKLMEWTSSNVMEERT |
| | | MKVYLPHMRMEEKYNLTSVLMALGVTDLFSSSANLSGISSAESLKMSEAVHEAF |
| | | VEIYESGSQVVGSTGAGTEVTSVSEEFRVDHPFLFLIKHNPTNSILFFGRCFSP |
| Ovalbumin-like | 36 | MGSIGAASVEFCFDVFKELKVQHVNENIFYSPLSIISALSMVYLGARENTKAQIDK |
| [Falco | | VVHFDKIAGFGEAIESQCVTSASIHSLKDMFTQITKPSDNYSLSFASRLYAEEAYSI |
| peregrinus] | | LPEYLQCVKELYKGGLETISFQTAADQARDLINSWVESQTNGMIKNILQPGAVDL |
| | | ETEMVLVNAIYFKGMWEKAFKDEDTQTVPFRMTEQESKPVQMMYQVGSFKVA |
| | | VMASDKIKILELPYASGQLSMVVVLPDDVSGLEQLEASITSEKLMEWTSSSIMEEK |
| | | KIKVYFPHMKIEEKYNLTSVLMALGMTDLFSSSANLSGISSAEKLKVSEAVHEAF |
| | | VEISEAGSEVVGSTEAGTEVTSVSEEFKADHPFLFLIKHNPTNSILFFGRCFSP |
| PREDICTED: | 37 | MGSIGAASSEFCFDIFKELKVQHVNENIFYSPLSIISALSMVYLGARENTRAQIDKV |
| Ovalbumin -like | | VPFDKITASGESIESQCSTSVSVHTSLKDIFTQITKSSDNHSLSFASRLYAEETYPILP |
| isoform X2 | | EYLQCVKELYEGGLETISFQTAADQARELINSWIESQTNGRIKNILQPGSVDPQTE |
| [Phalacrocorax | | MVLVNAIYFKGMWEKAFKDEDTQAVPFRMTEQESKPVQVMHQIGSFKVAVLAS |
| carbo] | | EKIKILELPYASGELSMLVLLPDDVSGLEQLETAITFEKLMEWTSPNIMEERKIKVF |
| | | LPRMKIEEKYNLTSVLMALGITDLFSPLANLSGISSAESLKMSEAIHEAFVEISEAG |
| | | SEVIGSTEAEVEVTNDPEEFRADHPFLFLIKHNPTNSILFFGRCFSP |
| PREDICTED: | 38 | MGSIGAASTEFCFDVFKELKAQYVNENIFYSPMTIITALSMVYLGSKENTRAQIAK |
| Ovalbumin-like | | VAHFDKITGFGESIESQCGASASIQFSLKDLFTQITKPSGNHSLSVASRIYAEETYPI |
| [Merops | | LPEYLECMKELYKGGLETINFQTAANQARELINSWVERQTSGMIKNILQPSSVDS |
| nubicus] | | QTEMVLVNAIYFRGLWEKAFKVEDTQATPFRITEQESKPVQMMHQIGSFKVAVV |
| | | ASEKIKILELPYASGRLTMLVVLPDDVSGLKQLETTITFEKLMEWTTSNIMEERKI |
| | | KVYLPRMKIEEKYNLTSVLMALGLTDLFSSSANLSGISSAESLKMSEAVHEAFVEI |
| | | YEAGSEVVASAEAGMDATSVSEEFRADHPFLFLIKDNTSNSILFFGRCFSP |
| PREDICTED: | 39 | MGSIGAASTEFCFDVFKELKGQHVNENIFFCPLSIVSALSMVYLGARENTRAQIVK |
| Ovalbumin-like | | VAHFDKIAGFAESIESQCGTSVSIHTSLKDMFTQITKPSDNYSLNFASRLYAEETYP |
| [Tauraco | | IIPEYLQCVKELYKGGLETISFQTAADQAREIINSWVESQTNGMIKNILRPSSVHPQ |
| erythrolophus] | | TELVLVNAVYFKGTWEKAFKDEDTQAVPFRITEQESKPVQMMYQIGSFKVAAVT |
| | | SEKMKILEVPYASGELSMLVLLPDDVSGLEQLETAITAEKLIEWTSSTVMEERKLK |
| | | VYLPRMKIEEKYNLTTVLTALGVTDLFSSSANLSGISSAQGLKMSNAVHEAFVEIY |
| | | EAGSEVVGSKGEGTEVSSVSDEFKADHPFLFLIKHNPTNSIVFFGRCFSP |
| PREDICTED: | 40 | MGSIGAASTEFCFDVFKELKVHHVNENILYSPLAIISALSMVYLGAKENTRDQIDK |
| Ovalbumin-like | | VVHFDKITGIGESIESQCSTAVSVHTSLKDVFDQITRPSDNYSLAFASRLYAEKTYP |
| [Cuculus | | ILPEYLQCVKELYKGGLETIDFQTAADQARQLINSWVEDETNGMIKNILRPSSVNP |
| canorus] | | QTKIILVNAIYFKGMWEKAFKDEDTQEVPFRITEQETKSVQMMYQIGSFKVAEVV |
| | | SDKMKILELPYASGKLSMLVLLPDDVYGLEQLETVITVEKLKEWTSSIVMEERITK |
| | | VYLPRMKIMEKYNLTSVLTAFGITDLFSPSANLSGISSTESLKVSEAVHEAFVEIHE |
| | | AGSEVVGSAGAGIEATSVSEEFKADHPFLFLIKHNPTNSILFFGRCFSP |
| Ovalbumin | 41 | MGSIGAASTEFCLDVFKELKVQHVNENIFYSPLSIISALSMVYLGARENTRAQIDK |
| [Antrostomus | | VVHFDKITGFEDSIESQCGTSVSVHTSLKDMFTQITKPSDNYSVGFASRLYAAETY |
| carolinensis] | | QILPEYSQCVKELYKGGLETINFQKAADQATELINSWVESQTNGMIKNILQPSSVD |
| | | PQTQIFLVNAIYFKGMWQRAFKEEDTQAVPFRISEKESKPVQMMYQIGSFKVAVI |
| | | PSEKIKILELPYASGLLSMLVILPDDVSGLEQLENAITLEKLMQWTSSNMMEERKI |
| | | KVYLPRMRMEEKYNLTSVFMALGITDLFSSSANLSGISSAESLKMSDAVHEASVEI |
| | | HEAGSEVVGSTGSGTEASSVSEEFRADHPYLFLIKHNPTDSIVFFGRCFSP |
| PREDICTED: | 42 | MGSIGAASTEFCFDVFKELKFQHVDENIFYSPLTIISALSMVYLGARENTRAQIDK |
| Ovalbumin-like | | VVHFDKIAGFEETVESQCGTSVSVHTSLKDMFAQITKPSDNYSLSFASRLYAEETY |
| [Opisthocomus | | PILPEYLQCVKELYKGGLETISFQTAADQARDLINSWVESQTNGMIKNILQPSSVG |
| hoazin] | | PQTELILVNAIYFKGMWQKAFKDEDTQEVPFRMTEQQSKPVQMMYQTGSFKVA |
| | | VVASEKMKILALPYASGQLSLLVMLPDDVSGLKQLESAITSEKLIEWTSPSMMEE |
| | | RKIKVYLPRMKIEEKYNLTSVLMALGITDLFSPSANLSGISSAESLKMSQAVHEAF |
| | | VEIYEAGSEVVGSTGAGMEDSSDSEEFRVDHPFLFFIKHNPTNSILFFGRCFSP |
| PREDICTED: | 43 | MGSIGPLSVEFCCDVFKELRIQHPRENIFYSPVTIISALSMVYLGARDNTKAQIEKA |
| Ovalbumin-like | | VHFDKIPGFGESIESQCGTSLSIHTSLKDIFTQITKPSDNYTVGIASRLYAEEKYPILP |
| [Lepidothrix | | EYLQCIKELYKGGLEPINFQTAAEQARELINSWVESQTNGMIKNILQPSSVNPETD |
| coronata] | | MVLVNAIYFKGLWEKAFKDEDIQTVPFRITEQESKPVQMMFQIGSFRVAEITSEKI |
| | | RILELPYASGQLSLWVLLPDDISGLEQLETAITFENLKEWTSSTKMEERKIKVYLPR |
| | | MKIEEKYNLTSVLTSLGITDLFSSSANLSGISSAESLKVSSAFHEASVEIYEAGSKV |
| | | VGSTGAEVEDTSVSEEFRADHPFLFLIKHNPSNSIFFFGRCFSP |
| PREDICTED: | 44 | MGSIGTASAEFCFDVFKELKVHHVNENIFYSPLSIISALSMVYLGARENTKTQMEK |
| Ovalbumin | | VIHFDKITGLGESMESQCGTGVSIHTALKDMLSEITKPSDNYSLSLASRLYAEQTY |
| [Struthio | | AILPEYLQCIKELYKESLETVSFQTAADQARELINSWIESQTNGVIKNFLQPGSVDS |
| camelus | | QTELVLVNAIYFKGMWEKAFKDEDTQEVPFRITEQESRPVQMMYQAGSFKVATV |
| australis] | | AAEKIKILELPYASGELSMLVLLPDDISGLEQLETTISFEKLTEWTSSNMMEDRNM |
| | | KVYLPRMKIEEKYNLTSVLIALGMTDLFSPAANLSGISAAESLKMSEAIHAAYVEI |
| | | YEADSEIVSSAGVQVEVTSDSEEFRVDHPFLFLIKHNPTNSVLFFGRCISP |
| PREDICTED: | 45 | MGSIGAVSTEFSCDVFKELRIHHVQENIFYSPVTIISALSMIYLGARDSTKAQIEKA |
| Ovalbumin-like | | VHFDKIPGFGESIESQCGTSLSIHTSIKDMFTKITKASDNYSIGIASRLYAEEKYPILP |
| [Acanthisitta | | EYLQCVKELYKGGLESISFQTAAEQAREIINSWVESQTNGMIKNILQPSSVDPQTDI |
| chloris] | | VLVNAIYFKGLWEKAFRDEDTQTVPFKITEQESKPVQMMYQIGSFKVAEITSEKIK |
| | | ILEVPYASGQLSLWVLLPDDISGLEKLETAITFENLKEWTSSTKMEERKIKVYLPR |
| | | MKIEEKYNLTSVLTALGITDLFSSSANLSGISSAESLKVSEAFHEAIVEISEAGSKVV |
| | | GSVGAGVDDTSVSEEFRADHPFLFLIKHNPTSSIFFFGRCFSP |
| PREDICTED: | 46 | MGSIGAASTEFCFDVFKELKVQHVNENIFYSPLSIISALSMVYLGARENTRAQIDK |
| Ovalbumin-like | | VVHFDKIAGFGESTESQCGTSVSAHTSLKDMSNQITKLSDNYSLSFASRLYAEETY |
| [Tyto alba] | | PILPEYSQCVKELYKGGLESISFQTAAYQARELINAWVESQTNGMIKDILQPGSVD |
| | | SQTKMVLVNAIYFKGIWEKAFKDEDTQEVPFRMTEQETKPVQMMYQIGSFKVAV |
| | | IAAEKIKILELPYASGQLSMLVILPDDVSGLEQLETAITFEKLTEWTSASVMEERKI |
| | | KVYLPRMSIEEKYNLTSVLIALGVTDLFSSSANLSGISSAESLRMSEAIHEAFVETY |
| | | EAGSTESGTEVTSASEEFRVDHPFLFLIKHKPTNSILFFGRCFSP |
| PREDICTED: | 47 | MGSIGAASSEFCFDIFKELKVQHVNENIFYSPLSIISALSMVYLGARENTRAQIDKV |
| Ovalbumin -like | | VPFDKITASGESIESQVQKIQCSTSVSVHTSLKDIFTQITKSSDNHSLSFASRLYAEE |
| isoform XI | | TYPILPEYLQCVKELYEGGLETISFQTAADQARELINSWIESQTNGRIKNILQPGSV |
| [Phalacrocorax | | DPQTEMVLVNAIYFKGMWEKAFKDEDTQAVPFRMTEQESKPVQVMHQIGSFKV |
| carbo] | | AVLASEKIKILELPYASGELSMLVLLPDDVSGLEQLETAITFEKLMEWTSPNIMEE |
| | | RKIKVFLPRMKIEEKYNLTSVLMALGITDLFSPLANLSGISSAESLKMSEAIHEAFV |
| | | EISEAGSEVIGSTEAEVEVTNDPEEFRADHPFLFLIKHNPTNSILFFGRCFSP |
| Ovalbumin-like | 48 | MGSIGPLSVEFCCDVFKELRIQHARENIFYSPVTIISALSMVYLGARDNTKAQIEKA |
| [Pipra filicauda] | | VHFDKIPGFGESIESQCGTSLSIHTSLKDIFTQITKPSDNYTVGIASRLYAEEKYPILP |
| | | EYLQCIKELYKGGLEPISFQTAAEQARELINSWVESQTNGIIKNILQPSSVNPETDM |
| | | VLVNAIYFKGLWEKAFKDEGTQTVPFRITEQESKPVQMMFQIGSFRVAEIASEKIR |
| | | ILELPYASGQLSLWVLLPDDISGLEQLETAITFENLKEWTSSTKMEERKIKVYLPR |
| | | MKIEEKYNLTSVLTSLGITDLFSSSANLSGISSAERLKVSSAFHEASMEINEAGSKV |
| | | VGAGVDDTSVSEEFRVDRPFLFLIKHNPSNSIFFFGRCFSP |
| Ovalbumin | 49 | MGSIGAASTEFCFDMFKELKVHHVNENIIYSPLSIISILSMVFLGARENTKTQMEKV |
| [Dromaius | | IHFDKITGFGESLESQCGTSVSVHASLKDILSEITKPSDNYSLSLASKLYAEETYPVL |
| novaehollandiae] | | PEYLQCIKELYKGSLETVSFQTAADQARELINSWVETQTNGVIKNFLQPGSVDPQT |
| | | EMVLVDAIYFKGTWEKAFKDEDTQEVPFRITEQESKPVQMMYQAGSFKVATVA |
| | | AEKMKILELPYASGELSMFVLLPDDISGLEQLETTISIEKLSEWTSSNMMEDRKMK |
| | | VYLPHMKIEEKYNLTSVLVALGMTDLFSPSANLSGISTAQTLKMSEAIHGAYVEIY |
| | | EAGSEMATSTGVLVEAASVSEEFRVDHPFLFLIKHNPSNSILFFGRCIFP |
| Chain A, | 50 | MGSIGAASTEFCFDMFKELKVHHVNENIIYSPLSIISILSMVFLGARENTKTQMEKV |
| Ovalbumin | | IHFDKITGFGESLESQCGTSVSVHASLKDILSEITKPSDNYSLSLASKLYAEETYPVL |
| | | PEYLQCIKELYKGSLETVSFQTAADQARELINSWVETQTNGVIKNFLQPGSVDPQT |
| | | EMVLVDAIYFKGTWEKAFKDEDTQEVPFRITEQESKPVQMMYQAGSFKVATVA |
| | | AEKMKILELPYASGELSMFVLLPDDISGLEQLETTISIEKLSEWTSSNMMEDRKMK |
| | | VYLPHMKIEEKYNLTSVLVALGMTDLFSPSANLSGISTAQTLKMSEAIHGAYVEIY |
| | | EAGSEMATSTGVLVEAASVSEEFRVDHPFLFLIKHNPSNSILFFGRCIFPHHHHHH |
| Ovalbumin-like | 51 | MGSIGPLSVEFCCDVFKELRIQHARENIFYSPVTIISALSMVYLGARDNTKAQIEKA |
| [Corapipo | | VHFDKIPGFGESIESQCGTSLSIHTSLKDIFTQITKPSDNYTVGIASRLYAEEKYPILP |
| altera] | | EYLQCIKELYKGGLEPISFQTAAEQARELINSWVESQTNGMIKNILQPSAVNPETD |
| | | MVLVNAIYFKGLWEKAFKDEGTQTVPFRITEQESKPVQMMFQIGSFRVAEITSEKI |
| | | RILELPYASGQLSLWVLLPDDISGLEQLETAITFENLKEWTSSTKMEERKIKVYLPR |
| | | MKIEEKYNLTSVLTSLGITDLFSSSANLSGISSAERLKVSSAFHEASMEIYEAGSKV |
| | | VGSTGAGVDDTSVSEEFRVDRPFLFLIKHNPSNSIFFFGRCFSP |
| Ovalbumin-like | 52 | MEDQRGNTGFTMGSIGAASTEFCIDVFRELRVQHVNENIFYSPLTIISALSMVYLG |
| protein | | ARENTRAQIDQVVHFDKIAGFGDTVESQCGSSPSVHNSLKTVXAQITQPRDNYSL |
| [Amazona | | NLASRLYAEESYPILPEYLQCVKELYNGGLETVSFQTAADQARELINSWVESQTN |
| aestiva] | | GIIKNILQPSSVDPQTEMVLVNAIYFKGLWEKAFKDEETQAVPFRITEQENRPVQM |
| | | MYQFGSFKVAXVASEKIKILELPYASGQLSMLVLLPDEVSGLEQNAITFEKLTEW |
| | | TSSDLMEERKIKVFFPRVKIEEKYNLTAVLVSLGITDLFSSSANLSGISSAENLKMS |
| | | EAVHEAXVEIYEAGSEVAGSSGAGIEVASDSEEFRVDHPFLFLIXHNPTNSILFFGR |
| | | CFSP |
| PREDICTED: | 53 | MGSIGAASTEFCIDVFRELRVQHVNENIFYSPLSIISALSMVYLGARENTRAQIDEV |
| Ovalbumin-like | | FHFDKIAGFGDTVDPQCGASLSVHKSLQNVFAQITQPKDNYSLNLASRLYAEESY |
| [Melopsittacus | | PILPEYLQCVKELYNEGLETVSFQTGADQARELINSWVENQTNGVIKNILQPSSVD |
| undulatus] | | PQTEMVLVNAIYFKGLWQKAFKDEETQAVPFRITEQENRPVQMMYQFGSFKVAV |
| | | VASEKVKILELPYASGQLSMWVLLPDEVSGLEQLENAITFEKLTEWTSSDLTEER |
| | | KIKVFLPRVKIEEKYNLTAVLMALGVTDLFSSSANFSGISAAENLKMSEAVHEAF |
| | | VEIYEAGSEVVGSSGAGIEAPSDSEEFRADHPFLFLIKHNPTNSILFFGRCFSP |
| Ovalbumin-like | 54 | MGSIGPLSVEFCCDVFKELRIQHARDNIFYSPVTIISALSMVYLGARDNTKAQIEKA |
| [Neopelma | | VHFDKIPGFGESIESQCGTSLSVHTSLKDIFTQITKPRENYTVGIASRLYAEEKYPIL |
| chrysocephalum] | | PEYLQCIKELYKGGLEPISFQTAAEQARELINSWVESQTNGMIKNILQPSSVNPETD |
| | | MVLVNAIYFKGLWKKAFKDEGTQTVPFRITEQESKPVQMMFQIGSFRVAEITSEKI |
| | | RILELPYASGQLSLWVLLPDDISGLEQLESAITFENLKEWTSSTKMEERKIKVYLPR |
| | | MKIEEKYNLTSVLTSLGITDLFSSSANLSGISSAEKLKVSSAFHEASMEIYEAGNKV |
| | | VGSTGAGVDDTSVSEEFRVDRPFLFLIKHNPSNSIFFFGRCFSP |
| PREDICTED: | 55 | MGSIGAASAEFCVDVFKELKDQHVNNIVFSPLMIISALSMVNIGAREDTRAQIDKV |
| Ovalbumin-like | | VHFDKITGYGESIESQCGTSIGIYFSLKDAFTQITKPSDNYSLSFASKLYAEETYPIL |
| [Buceros | | PEYLKCVKELYKGGLETISFQTAADQARELINSWVESQTNGMIKNILQPSSVDPQT |
| rhinoceros | | EMVLVNAIYFKGLWEKAFKDEDTQAVPFRITEQESKPVQMMYQIGSFKVAVIASE |
| silvestris] | | KIKILELPYASGQLSLLVLLPDDVSGLEQLESAITSEKLLEWTNPNIMEERKTKVYL |
| | | PRMKIEEKYNLTSVLVALGITDLFSSSANLSGISSAEGLKLSDAVHEAFVEIYEAGR |
| | | EVVGSSEAGVEDSSVSEEFKADRPFIFLIKHNPTNGILYFGRYISP |
| PREDICTED: | 56 | MGSIGAANTDFCFDVFKELKVHHANENIFYSPLSIVSALAMVYLGARENTRAQID |
| Ovalbumin-like | | KALHFDKILGFGETVESQCDTSVSVHTSLKDMLIQITKPSDNYSFSFASKIYTEETY |
| [Cariama | | PILPEYLQCVKELYKGGVETISFQTAADQAREVINSWVESHTNGMIKNILQPGSVD |
| cristata] | | PQTKMVLVNAVYFKGIWEKAFKEEDTQEMPFRINEQESKPVQMMYQIGSFKLTV |
| | | AASENLKILEFPYASGQLSMMVILPDEVSGLKQLETSITSEKLIKWTSSNTMEERKI |
| | | RVYLPRMKIEEKYNLKSVLMALGITDLFSSSANLSGISSAESLKMSEAVHEAFVEI |
| | | YEAGSEVTSSTGTEMEAENVSEEFKADHPFLFLIKHNPTDSIVFFGRCMSP |
| Ovalbumin | 57 | MGSIGPLSVEFCCDVFKELRIQHARENIFYSPVTIISALSMVYLGARDNTKAQIEKA |
| [Manacus | | VHFDKIPGFGESIESQCGTSLSIHTSLKDIFTQITKPSDNYTVGIASRLYAEEKYPILP |
| vitellinus] | | EYLQCIKELYKGGLEPISFQTAAEQARELINSWVESQTNGMIKNILQPSSVNPETD |
| | | MVLVNAIYFKGLWEKAFKDESTQTVPFRITEQESKPVQMMFQIGSFRVAEIASEKI |
| | | RILELPYASGQLSLWVLLPDDISGLEQLETAITFENLKEWTSSTKMEERKIKVYLPR |
| | | MKIEEKYNLTSVLTSLGITDLFSSSANLSGISSAERLKVSSAFHEASMEIYEAGSRV |
| | | VEAGVDDTSVSEEFRVDRPFLFLIKHNPSNSIFFFGRCFSP |
| Ovalbumin-like | 58 | MGSIGPVSTEFCCDIFKELRIQHARENIIYSPVTIISALSMVYLGARDNTKAQIEKAV |
| [Empidonax | | HFDKIPGFGESIESQCGTSLSIHTSLKDILTQITKPSDNYTVGIASRLYAEEKYPILSE |
| traillii] | | YLQCIKELYKGGLEPISFQTAAEQARELINSWVESQTNGMIKNILQPSSVNPETDM |
| | | VLVNAIYFKGLWEKAFKDEGTQTVPFRITEQESKPVQMMFQIGSFKVAEITSEKIR |
| | | ILELPYASGKLSLWVLLPDDISGLEQLETAITFENLKEWTSSTRMEERKIKVYLPR |
| | | MKIEEKYNLTSVLTSLGITDLFSSSANLSGISSAERLKVSSAFHEVFVEIYEAGSKV |
| | | EGSTGAGVDDTSVSEEFRADHPFLFLVKHNPSNSIIFFGRCYLP |
| PREDICTED: | 59 | MGSTGAASMEFCFALFRELKVQHVNENIFFSPVTIISALSMVYLGARENTRAQLD |
| Ovalbumin-like | | KVAPFDKITGFGETIGSQCSTSASSHTSLKDVFTQITKASDNYSLSFASRLYAEETY |
| [Leptosomus | | PILPEYLQCVKELYKGGLESISFQTAADQARELINSWVESQTNGMIKDILRPSSVDP |
| discolor] | | QTKIILITAIYFKGMWEKAFKEEDTQAVPFRMTEQESKPVQMMYQIGSFKVAVIPS |
| | | EKLKILELPYASGQLSMLVILPDDVSGLEQLETAITTEKLKEWTSPSMMKERKMK |
| | | VYFPRMRIEEKYNLTSVLMALGITDLFSPSANLSGISSAESLKVSEAVHEASVDIDE |
| | | AGSEVIGSTGVGTEVTSVSEEIRADHPFLFLIKHKPTNSILFFGRCFSP |
| Hypothetical | 60 | MEHAQLTQLVNSNMTSNTCHEADEFENIDFRMDSISVTNTKFCFDVFNEMKVHH |
| protein | | VNENILYSPLSILTALAMVYLGARGNTESQMKKALHFDSITGAGSTTDSQCGSSE |
| H355_008077 | | YIHNLFKEFLTEITRTNATYSLEIADKLYVDKTFTVLPEYINCARKFYTGGVEEVN |
| [Colinus | | FKTAAEEARQLINSWVEKETNGQIKDLLVPSSVDFGTMMVFINTIYFKGIWKTAF |
| virginianus] | | NTEDTREMPFSMTKQESKPVQMMCLNDTFNMATLPAEKMRILELPYASGELSML |
| | | VLLPDEVSGLEQIEKAINFEKLREWTSTNAMEKKSMKVYLPRMKIEEKYNLTSTL |
| | | MALGMTDLFSRSANLTGISSVENLMISDAVHGAFMEVNEEGTEAAGSTGAIGNIK |
| | | HSVEFEEFRADHPFLFLIRYNPTNVILFFDNSEFTMGSIGAVSTEFCFDVFKELRVH |
| | | HANENIFYSPFTVISALAMVYLGAKDSTRTQINKVVRFDKLPGFGDSIEAQCGTSA |
| | | NVHSSLRDILNQITKPNDIYSFSLASRLYADETYTILPEYLQCVKELYRGGLESINF |
| | | QTAADQARELINSWVESQTSGIIRNVLQPSSVDSQTAMVLVNAIYFKGLWEKGFK |
| | | DEDTQAMPFRVTEQENKSVQMMYQIGTFKVASVASEKMKILELPFASGTMSMW |
| | | VLLPDEVSGLEQLETTISIEKLTEWTSSSVMEERKIKVFLPRMKMEEKYNLTSVLM |
| | | AMGMTDLFSSSANLSGISSTLQKKGFRSQELGDKYAKPMLESPALTPQVTAWDN |
| | | SWIVAHPAAIEPDLCYQIMEQKWKPFDWPDFRLPMRVSCRFRTMEALNKANTSF |
| | | ALDFFKHECQEDDDENILFSPFSISSALATVYLGAKGNTADQMAKTEIGKSGNIHA |
| | | GFKALDLEINQPTKNYLLNSVNQLYGEKSLPFSKEYLQLAKKYYSAEPQSVDFLG |
| | | KANEIRREINSRVEHQTEGKIKNLLPPGSIDSLTRLVLVNALYFKGNWATKFEAED |
| | | TRHRPFRINMHTTKQVPMMYLRDKFNWTYVESVQTDVLELPYVNNDLSMFILLP |
| | | RDITGLQKLINELTFEKLSAWTSPELMEKMKMEVYLPRFTVEKKYDMKSTLSKM |
| | | GIEDAFTKVDSCGVTNVDEITTHIVSSKCLELKHIQINKKLKCNKAVAMEQVSASI |
| | | GNFTIDLFNKLNETSRDKNIFFSPWSVSSALALTSLAAKGNTAREMAEDPENEQA |
| | | ENIHSGFKELMTALNKPRNTYSLKSANRIYVEKNYPLLPTYIQLSKKYYKAEPYK |
| | | VNFKTAPEQSRKEINNWVEKQTERKIKNFLSSDDVKNSTKSILVNAIYFKAEWEE |
| | | KFQAGNTDMQPFRMSKNKSKLVKMMYMRHTFPVLIMEKLNFKMIELPYVKREL |
| | | SMFILLPDDIKDSTTGLEQLERELTYEKLSEWADSKKMSVTLVDLHLPKFSMEDR |
| | | YDLKDALKSMGMASAFNSNADFSGMTGFQAVPMESLSASTNSFTLDLYKKLDET |
| | | SKGQNIFFASWSIATALAMVHLGAKGDTATQVAKGPEYEETENIHSGFKELLSAI |
| | | NKPRNTYLMKSANRLFGDKTYPLLPKFLELVARYYQAKPQAVNFKTDAEQARA |
| | | QINSWVENETESKIQNLLPAGSIDSHTVLVLVNAIYFKGNWEKRFLEKDTSKMPF |
| | | RLSKTETKPVQMMFLKDTFLIHHERTMKFKIIELPYVGNELSAFVLLPDDISDNTT |
| | | GLELVERELTYEKLAEWSNSASMMKAKVELYLPKLKMEENYDLKSVLSDMGIRS |
| | | AFDPAQADFTRMSEKKDLFISKVIHKAFVEVNEEDRIVQLASGRLTGRCRTLANK |
| | | ELSEKNRTKNLFFSPFSISSALSMILLGSKGNTEAQIAKVLSLSKAEDAHNGYQSLL |
| | | SEINNPDTKYILRTANRLYGEKTFEFLSSFIDSSQKFYHAGLEQTDFKNASEDSRKQ |
| | | INGWVEEKTEGKIQKLLSEGIINSMTKLVLVNAIYFKGNWQEKFDKETTKEMPFKI |
| | | NKNETKPVQMMFRKGKYNMTYIGDLETTVLEIPYVDNELSMIILLPDSIQDESTGL |
| | | EKLERELTYEKLMDWINPNMMDSTEVRVSLPRFKLEENYELKPTLSTMGMPDAF |
| | | DLRTADFSGISSGNELVLSEVVHKSFVEVNEEGTEAAAATAGIMLLRCAMIVANF |
| | | TADHPFLFFIRHNKTNSILFCGRFCSP |
| PREDICTED: | 61 | MGSIGTASTEFCFDMFKEMKVQHANQNIIFSPLTIISALSMVYLGARDNTKAQME |
| Ovalbumin | | KVIHFDKITGFGESVESQCGTSVSIHTSLKDMLSEITKPSDNYSLSLASRLYAEETY |
| isoform X2 | | PILPEYLQCMKELYKGGLETVSFQTAADQARELINSWVESQTNGVIKNFLQPGSV |
| [Apteryx | | DPQTEMVLVNAIYFKGMWEKAFKDEDTQEVPFRITEQESKPVQMMYQVGSFKV |
| australis | | ATVAAEKMKILEIPYTHRELSMFVLLPDDISGLEQLETTISFEKLTEWTSSNMMEE |
| mantelli] | | RKVKVYLPHMKIEEKYNLTSVLMALGMTDLFSPSANLSGISTAQTLMMSEAIHG |
| | | AYVEIYEAGREMASSTGVQVEVTSVLEEVRADKPFLFFIRHNPTNSMVVFGRYMS |
| | | P |
| Hypothetical | 62 | MTSNTCHEADEFENIDFRMDSISVTNTKFCFDVFNEMKVHHVNENILYSPLSILTA |
| protein | | LAMVYLGARGNTESQMKKALHFDSITGGGSTTDSQCGSSEYIHNLFKEFETEITRT |
| ASZ78_006007 | | NATYSLEIADKLYVDKTFTVLPEYINCARKFYTGGVEEVNFKTAAEEARQLMNS |
| [Callipepla | | WVEKETNGQIKDLLVPSSVDFGTMMVFINTIYFKGIWKTAFNTEDTREMPFSMTK |
| squamata] | | QESKPVQMMCLNDTFNMVTLPAEKMRILELPYASGELSMLVLLPDEVSGLERIEK |
| | | AINFEKLREWTSTNAMEKKSMKVYLPRMKIEEKYNLTSTLMALGMTDLFSRSAN |
| | | LTGISSVDNLMISDAVHGAFMEVNEEGTEAAGSTGAIGNIKHSVEFEEFRADHPFL |
| | | FLIRYNPTNVILFFDNSEFTMGSIGAVSTEFCFDVFKELRVHHANENIFYSPFTIISA |
| | | LAMVYLGAKDSTRTQINKVVRFDKLPGFGDSIEAQCGTSANVHSSLRDILNQITKP |
| | | NDIYSFSLASRLYADETYTILPEYLQCVKELYRGGLESINFQTAADQARELINSWV |
| | | ESQTSGIIRNVLQPSSVDSQTAMVLVNAIYFKGLWEKGFKDEDTQAIPFRVTEQEN |
| | | KSVQMMYQIGTFKVASVASEKMKILELPFASGTMSMWVLLPDEVSGLEQLETTIS |
| | | IEKLTEWTSSSVMEERKIKVFLPRMKMEEKYNLTSVLMAMGMTDLFSSSANESGI |
| | | SSTLQKKGFRSQELGDKYAKPMLESPALTPQATAWDNSWIVAHPPAIEPDLYYQI |
| | | MEQKWKPFDWPDFRLPMRVSCRFRTMEALNKANTSFALDFFKHECQEDDSENIL |
| | | FSPFSISSALATVYLGAKGNTADQMAKVLHFNEAEGARNVTTTIRMQVYSRTDQ |
| | | QRLNRRACFQKTEIGKSGNIHAGFKGLNLEINQPTKNYLLNSVNQLYGEKSLPFSK |
| | | EYLQLAKKYYSAEPQSVDFVGTANEIRREINSRVEHQTEGKIKNLLPPGSIDSLTRL |
| | | VLVNALYFKGNWATKFEAEDTRHRPFRINTHTTKQVPMMYLSDKFNWTYVESV |
| | | QTDVLELPYVNNDLSMFILLPRDITGLQKLINELTFEKLSAWTSPELMEKMKMEV |
| | | YLPRFTVEKKYDMKSTLSKMGIEDAFTKVDNCGVTNVDEITIHVVPSKCLELKHI |
| | | QINKELKCNKAVAMEQVSASIGNFTIDLFNKLNETSRDKNIFFSPWSVSSALALTS |
| | | LAAKGNTAREMAEDPENEQAENIHSGFNELLTALNKPRNTYSLKSANRIYVEKN |
| | | YPLLPTYIQLSKKYYKAEPHKVNFKTAPEQSRKEINNWVEKQTERKIKNFESSDD |
| | | VKNSTKLILVNAIYFKAEWEEKFQAGNTDMQPFRMSKNKSKEVKMMYMRHTFP |
| | | VLIMEKLNFKMIELPYVKRELSMFILLPDDIKDSTTGLEQLERELTYEKESEWADS |
| | | KKMSVTLVDLHLPKFSMEDRYDLKDALRSMGMASAFNSNADFSGMTGERDLVI |
| | | SKVCHQSFVAVDEKGTEAAAATAVIAEAVPMESLSASTNSFTLDLYKKLDETSKG |
| | | QNIFFASWSIATALTMVHLGAKGDTATQVAKGPEYEETENIHSGFKELLSALNKP |
| | | RNTYSMKSANRLFGDKTYPLLPTKTKPVQMMFLKDTFLIHHERTMKFKIIELPYM |
| | | GNELSAFVLLPDDISDNTTGLELVERELTYEKLAEWSNSASMMKVKVELYLPKL |
| | | KMEENYDLKSALSDMGIRSAFDPAQADFTRMSEKKDLFISKVIHKAFVEVNEEDR |
| | | IVQLASGRLTGNTEAQIAKVLSLSKAEDAHNGYQSLLSEINNPDTKYILRTANRLY |
| | | GEKTFEFLSSFIDSSQKFYHAGLEQTDFKNASEDSRKQINGWVEEKTEGKIQKELS |
| | | EGIINSMTKLVLVNAIYFKGNWQEKFDKETTKEMPFKINKNETKPVQMMFRKGK |
| | | YNMTYIGDLETTVLEIPYVDNELSMIILLPDSIQDESTGLEKLERELTYEKEMDWIN |
| | | PNMMDSTEVRVSLPRFKEEENYELKPTLSTMGMPDAFDLRTADFSGISSGNELVL |
| | | SEVVHKSFVEVNEEGTEAAAATAGIMLLRCAMIVANFTADHPFLFFIRHNKTNSIL |
| | | FCGRFCSP |
| PREDICTED: | 63 | MASIGAASTEFCFDVFKELKTQHVKENIFYSPMAIISALSMVYIGARENTRAEIDK |
| Ovalbumin-like | | VVHFDKITGFGNAVESQCGPSVSVHSSLKDLITQISKRSDNYSLSYASRIYAEETYP |
| [Mesitornis | | ILPEYLQCVKEVYKGGLESISFQTAADQARENINAWVESQTNGMIKNILQPSSVNP |
| unicolor] | | QTEMVLVNAIYLKGMWEKAFKDEDTQTMPFRVTQQESKPVQMMYQIGSFKVAV |
| | | IASEKMKILELPYTSGQLSMLVLLPDDVSGLEQVESAITAEKLMEWTSPSIMEERT |
| | | MKVYLPRMKMVEKYNLTSVLMALGMTDLFTSVANLSGISSAQGLKMSQAIHEA |
| | | FVEIYEAGSEAVGSTGVGMEITSVSEEFKADLSFLFLIRHNPTNSIIFFGRCISP |
| Ovalbumin, | 64 | MGSIGAASTEFCFDVFRELRVQHVNENIFYSPFSIISALAMVYLGARDNTRTQIDKI |
| partial [Anas | | SQFQALSDEHLVLCIQQLGEFFVCTNRERREVTRYSEQTEDKTQDQNTGQIHKIV |
| platyrhynchos] | | DTCMLRQDILTQITKPSDNFSLSFASRLYAEETYAILPEYLQCVKELYKGGLESISF |
| | | QTAADQARELINSWVESQTNGIIKNILQPSSVDSQTTMVLVNAIYFKGMWEKAFK |
| | | DEDTQAMPFRMTEQESKPVQMMYQVGSFKVAMVTSEKMKILELPFASGMMSMF |
| | | VLLPDEVSGLEQLESTISFEKLTEWTSSTMMEERRMKVYLPRMKMEEKYNLTSVF |
| | | MALGMTDLFSSSANMSGISSTVSLKMSEAVHAACVEIFEAGRDVVGSAEAGMDV |
| | | TSVSEEFRADHPFLFFIKHNPTNSILFFGRWMSP |
| PREDICTED: | 65 | MGSIGAASAEFCLDIFKELKVQHVNENIIFSPMTIISALSLVYLGAKEDTRAQIEKV |
| Ovalbumin-like | | VPFDKIPGFGEIVESQCPKSASVHSSIQDIFNQIIKRSDNYSLSLASRLYAEESYPIRP |
| [Chaetura | | EYLQCVKELDKEGLETISFQTAADQARQLINSWVESQTNGMIKNILQPSSVNSQTE |
| pelagica] | | MVLVNAIYFRGLWQKAFKDEDTQAVPFRITEQESKPVQMMQQIGSFKVAEIASE |
| | | KMKILELPYASGQLSMLVLLPDDVSGLEKLESSITVEKLIEWTSSNLTEERNVKVY |
| | | LPRLKIEEKYNLTSVLAALGITDLFSSSANLSGISTAESLKLSRAVHESFVEIQEAGH |
| | | EVEGPKEAGIEVTSALDEFRVDRPFLFVTKHNPTNSILFLGRCLSP |
| PREDICTED: | 66 | MGSISAASGEFCLDIFKELKVQHVNENIFYSPMVIVSALSLVYLGARENTRAQIDK |
| Ovalbumin-like | | VIPFDKITGSSEAVESQCGTPVGAHISLKDVFAQIAKRSDNYSLSFVNRLYAEETYP |
| [Apaloderma | | ILPEYLQCVKELYKGGLETISFQTAADQAREIINSWVESQTDGKIKNILQPSSVDPQ |
| vittatum] | | TKMVLVSAIYFKGLWEKSFKDEDTQAVPFRVTEQESKPVQMMYQIGSFKVAAIA |
| | | AEKIKILELPYASEQLSMLVLLPDDVSGLEQLEKKISYEKLTEWTSSSVMEEKKIK |
| | | VYLPRMKIEEKYNLTSILMSLGITDLFSSSANLSGISSTKSLKMSEAVHEASVEIYE |
| | | AGSEASGITGDGMEATSVFGEFKVDHPFLFMIKHKPTNSILFFGRCISP |
| Ovalbumin-like | 67 | MGSIGPVSTEVCCDIFRELRSQSVQENVCYSPLLIISTLSMVYIGAKDNTKAQIEKA |
| [Corvus cornix | | IHFDKIPGFGESTESQCGTSVSIHTSLKDIFTQITKPSDNYSISIARRLYAEEKYPILPE |
| cornix] | | YIQCVKELYKGGLESISFQTAAEKSRELINSWVESQTNGTIKNILQPSSVSSQTDMV |
| | | LVSAIYFKGLWEKAFKEEDTQTIPFRITEQESKPVQMMSQIGTFKVAEIPSEKCRIL |
| | | ELPYASGRLSLWVLLPDDISGLEQLETAITFENLKEWTSSSKMEERKIRVYLPRMK |
| | | IEEKYNLTSVLKSLGITDLFSSSANLSGISSAESLKVSAAFHEASVEIYEAGSKGVG |
| | | SSEAGVDGTSVSEEIRADHPFLFLIKHNPSDSILFFGRCFSP |
| PREDICTED: | 68 | MGSIGAASTEFCFDVFKELKVQHVNENIIISPLSIISALSMVYLGAREDTRAQIDKV |
| Ovalbumin-like | | VHFDKITGFGEAIESQCPTSESVHASLKETFSQLTKPSDNYSLAFASRLYAEETYPI |
| [Calypte anna] | | LPEYLQCVKELYKGGLETINFQTAAEQARQVINSWVESQTDGMIKSLLQPSSVDP |
| | | QTEMILVNAIYFRGLWERAFKDEDTQELPFRITEQESKPVQMMSQIGSFKVAVVA |
| | | SEKVKILELPYASGQLSMLVLLPDDVSGLEQLESSITVEKLIEWISSNTKEERNIKV |
| | | YLPRMKIEEKYNLTSVLVALGITDLFSSSANLSGISSAESLKISEAVHEAFVEIQEA |
| | | GSEVVGSPGPEVEVTSVSEEWKADRPFLFLIKHNPTNSILFFGRYISP |
| PREDICTED: | 69 | MGSIGPVSTEVCCDIFRELRSQSVQENVCYSPLLIISTLSMVYIGAKDNTKAQIEKA |
| Ovalbumin | | IHFDKIPGFGESTESQCGTSVSIHTSLKDIFTQITKPSDNYSISIARRLYAEEKYPILQ |
| [Corvus | | EYIQCVKELYKGGLESISFQTAAEKSRELINSWVESQTNGTIKNILQPSSVSSQTDM |
| brachyrhynchos] | | VLVSAIYFKGLWEKAFKEEDTQTIPFRITEQESKPVQMMSQIGTFKVAEIPSEKCRI |
| | | LELPYASGRLSLWVLLPDDISGLEQLETSITFENLKEWTSSSKMEERKIRVYLPRM |
| | | KIEEKYNLTSVLKSLGITDLFSSSANLSGISSAESLKVSAVFHEASVEIYEAGSKGV |
| | | GSSEAGVDGTSVSEEIRADHPFLFLIKHNPSDSILFFGRCFSP |
| Hypothetical | 70 | MLNLMHPKQFCCTMGSIGPVSTEVCCDIFRELRSQSVQENVCYSPLLIISTLSMVYI |
| protein | | GAKDNTKAQIEKAIHFDKIPGFGESTESQCGTSVSIHTSLKDIFTQITKPSDNYSISIA |
| DUI87_08270 | | SRLYAEEKYPILPEYIQCVKELYKGGLESISFQTAAEKSRELINSWVESQTNGTIKN |
| [Hirundo rustica | | ILQPSSVSSQTDMVLVSAIYFKGLWEKAFKEEDTQTVPFRITEQESKPVQMMSQIG |
| rustica] | | TFKVAEIPSEKCRILELPYASGRLSLWVLLPDDISGLEQLETAITSENLKEWTSSSK |
| | | MEERKIKVYLPRMKIEEKYNLTSVLKSLGITDLFSSSANLSGISSAESLKVSGAFHE |
| | | AFVEIYEAGSKAVGSSGAGVEDTSVSEEIRADHPFLFFIKHNPSDSILFFGRCFSP |
| Ostrich OVA | 71 | EAEAGSIGTASAEFCFDVFKELKVHHVNENIFYSPLSIISALSMVYLGARENTKTQ |
| sequence as | | MEKVIHFDKITGLGESMESQCGTGVSIHTALKDMLSEITKPSDNYSLSLASRLYAE |
| secreted from | | QTYAILPEYLQCIKELYKESLETVSFQTAADQARELINSWIESQTNGVIKNFLQPGS |
| pichia | | VDSQTELVLVNAIYFKGMWEKAFKDEDTQEVPFRITEQESRPVQMMYQAGSFKV |
| | | ATVAAEKIKILELPYASGELSMLVLLPDDISGLEQLETTISFEKLTEWTSSNMMED |
| | | RNMKVYLPRMKIEEKYNLTSVLIALGMTDLFSPAANLSGISAAESLKMSEAIHAA |
| | | YVEIYEADSEIVSSAGVQVEVTSDSEEFRVDHPFLFLIKHNPTNSVLFFGRCISP |
| Ostrich construct | 72 | MRFPSIFTAVLFAASSALAAPVNTTTEDETAQIPAEAVIGYSDLEGDFDVAVLPFS |
| (secretion | | NSTNNGLLFINTTIASIAAKEEGVSLEKREAEAGSIGTASAEFCFDVFKELKVHHV |
| signal + mature | | NENIFYSPLSIISALSMVYLGARENTKTQMEKVIHFDKITGLGESMESQCGTGVSIH |
| protein) | | TALKDMLSEITKPSDNYSLSLASRLYAEQTYAILPEYLQCIKELYKESLETVSFQTA |
| | | ADQARELINSWIESQTNGVIKNFLQPGSVDSQTELVLVNAIYFKGMWEKAFKDED |
| | | TQEVPFRITEQESRPVQMMYQAGSFKVATVAAEKIKILELPYASGELSMLVLLPD |
| | | DISGLEQLETTISFEKLTEWTSSNMMEDRNMKVYLPRMKIEEKYNLTSVLIALGM |
| | | TDLFSPAANLSGISAAESLKMSEAIHAAYVEIYEADSEIVSSAGVQVEVTSDSEEFR |
| | | VDHPFLFLIKHNPTNSVLFFGRCISP |
| Duck OVA | 73 | EAEAGSIGAASTEFCFDVFRELRVQHVNENIFYSPFSIISALAMVYLGARDNTRTQI |
| sequence as | | DKVVHFDKLPGFGESMEAQCGTSVSVHSSLRDILTQITKPSDNFSLSFASRLYAEE |
| secreted from | | TYAILPEYLQCVKELYKGGLESISFQTAADQARELINSWVESQTNGIIKNILQPSSV |
| pichia | | DSQTTMVLVNAIYFKGMWEKAFKDEDTQAMPFRMTEQESKPVQMMYQVGSFK |
| | | VAMVTSEKMKILELPFASGMMSMFVLLPDEVSGLEQLESTISFEKLTEWTSSTMM |
| | | EERRMKVYLPRMKMEEKYNLTSVFMALGMTDLFSSSANMSGISSTVSLKMSEAV |
| | | HAACVEIFEAGRDVVGSAEAGMDVTSVSEEFRADHPFLFFIKHNPTNSILFFGRW |
| | | MSP |
| Duck construct | 74 | MRFPSIFTAVLFAASSALAAPVNTTTEDETAQIPAEAVIGYSDLEGDFDVAVLPFS |
| (secretion | | NSTNNGLLFINTTIASIAAKEEGVSLEKREAEAGSIGAASTEFCFDVFRELRVQHVN |
| signal + mature | | ENIFYSPFSIISALAMVYLGARDNTRTQIDKVVHFDKLPGFGESMEAQCGTSVSVH |
| protein) | | SSLRDILTQITKPSDNFSLSFASRLYAEETYAILPEYLQCVKELYKGGLESISFQTAA |
| | | DQARELINSWVESQTNGIIKNILQPSSVDSQTTMVLVNAIYFKGMWEKAFKDEDT |
| | | QAMPFRMTEQESKPVQMMYQVGSFKVAMVTSEKMKILELPFASGMMSMFVLLP |
| | | DEVSGLEQLESTISFEKLTEWTSSTMMEERRMKVYLPRMKMEEKYNLTSVFMAL |
| | | GMTDLFSSSANMSGISSTVSLKMSEAVHAACVEIFEAGRDVVGSAEAGMDVTSV |
| | | SEEFRADHPFLFFIKHNPTNSILFFGRWMSP |
| Chicken | 75 | MGSIGAASMEFCFDVFKELKVHHANENIFYCPIAIMSALAMVYLGAKDSTRTQIN |
| Ovalbumin | | KVVRFDKLPGFGDSIEAQCGTSVNVHSSLRDILNQITKPNDVYSFSLASRLYAEER |
| sequence with | | YPILPEYLQCVKELYRGGLEPINFQTAADQARELINSWVESQTNGIIRNVLQPSSV |
| possible | | DSQTAMVLVNAIVFKGLWEKAFKDEDTQAMPFRVTEQESKPVQMMYQIGLFRV |
| truncations | | ASMASEKMKILELPFASGTMSMLVLLPDEVSGLEQLESIINFEKLTEWTSSNVMEE |
| | | RKIKVYLPRMKMEEKYNLTSVLMAMGITDVFSSSANLSGISSAESLKISQAVHAA |
| | | HAEINEAGREVVGSAEAGVDAASVSEEFRADHPFLFCIKHIATNAVLFFGRCVSP |

Expression of rOVA in a host cell, for instance a Pichia species, a Saccharomyces species, a Trichoderma species, a Pseudomonas species may lead to an addition of one or more amino acids to the OVA sequence as part of post-transcriptional or post-translational modifications. Such amino acids may not be part of the native OVA sequences. For instance, expressing an OVA sequence in a Pichia species, such as Komagataella phaffii and Komagataella pastoris may lead to addition of one or more amino acids at the N-terminus or C-terminus. In some cases, four amino acids EAEA (SEQ ID NO: 76) is added to the N-terminus of the OVA sequence upon expression in a host cell as shown in SEQ ID NO:1. For example, chicken rOVA may be provided encoding SEQ ID NO: 1, and following expression and secretion, the rOVA has the amino acid sequence of SEQ ID NO: 2.

In some embodiments, the rOVA can be a non-naturally occurring variant of an OVA. Such variant can comprise one or more amino acid insertions, deletions, or substitutions relative to a native OVA sequence.

Such a variant can have at least 70%, 75%, 80%, 85%, 90%, 95%, 96%, 97%, 98%, or 99% sequence identity to SEQ ID NOs: 1-75. The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software, with BLAST being the preferable alignment algorithm. Those skilled in the art can determine appropriate parameters for measuring alignment, including any algorithms needed to achieve maximal alignment over the full-length of the sequences being compared.

Depending on the host organism used to express the rOVA, the rOVA can have a glycosylation, acetylation, or phosphorylation pattern different from wildtype OVA. For example, the rOVA herein may or may not be glycosylated, acetylated, or phosphorylated. In some embodiments, the rOVA may have an avian, non-avian, microbial, non-microbial, mammalian, or non-mammalian glycosylation, acetylation, or phosphorylation pattern.

In some cases, the rOVA may be deglycosylated (e.g., chemically, enzymatically, Endo-H, PNGase F, O-Glycosidase, Neuraminidase, β1-4 Galactosidase, β-N-acetylglucosaminidase), deacetylated (e.g., protein deacetylase, histone deacetylase, sirtuin), or dephosphorylated (e.g., acid phosphatase, lambda protein phosphatase, calf intestinal phosphatase, alkaline phosphatase). In some embodiments, deglycosylation, deacetylation or dephosphorylation may produce a protein that is more uniform or is capable of producing a composition with less variation.

In some embodiments, the rOVA is recombinantly expressed in a host cell. As used herein, a “host” or “host cell” denotes here any protein production host selected or genetically modified to produce a desired product. Illustrative hosts include fungi, such as filamentous fungi, as well as bacteria, yeast, plant, insect, and mammalian cells. In some embodiments, the host cell may be Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, or Trichoderma vireus. In some embodiments, the host cell can be an organism that is approved as generally regarded as safe by the U.S. Food and Drug Administration.

In some embodiments, the rOVA protein can be recombinantly expressed in yeast, filamentous fungi or a bacterium. In some embodiments, rOVA protein is recombinantly expressed in a Pichia species (Komagataella phaffii and Komagataella pastoris), a Saccharomyces species, a Trichoderma species, a Pseudomonas species or an E. coli species.

Expression of an rOVA can be provided by an expression vector, a plasmid, a nucleic acid integrated into the host genome or other means. For example, a vector for expression can include: (a) a promoter element, (b) a signal peptide, (c) an OVA sequence heterologous to the host cell, and (d) a terminator element.

Expression vectors that can be used for expression of OVA include those containing an expression cassette with elements (a), (b), (c) and (d). In some embodiments, the signal peptide (b) need not be included in the vector. In general, the expression cassette is designed to mediate the transcription of the transgene when integrated into the genome of a cognate host microorganism.

To aide in the amplification of the vector prior to transformation into the host microorganism, a replication origin (e) may be contained in the vector (such as PUC_ORIC and PUC (DNA2.0)). To aide in the selection of microorganism stably transformed with the expression vector, the vector may also include a selection marker (f) such as URA3 gene and Zeocin resistance gene (ZeoR). The expression vector may also contain a restriction enzyme site (g) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (e), (f), and (g). Other expression elements and vector element known to one of skill in the art can be used in combination or substituted for the elements described herein.

Illustrative promoter elements (a) may include, but are not limited to, a constitutive promoter, inducible promoter, and hybrid promoter. Promoters include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbhl), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof.

A signal peptide (b), also known as a signal sequence, targeting signal, localization signal, localization sequence, signal peptide, transit peptide, leader sequence, or leader peptide, may support secretion of a protein or polynucleotide. Extracellular secretion of a recombinant or heterologously expressed protein from a host cell may facilitate protein purification. A signal peptide may be derived from a precursor (e.g., prepropeptide, preprotein) of a protein. Signal peptides can be derived from a precursor of a protein other than the signal peptides in native OVA. An example of secretion protein is a S. cerevisiae alpha factor pre pro sequence shown bolded and underlined in SEQ ID NO: 1.

Any nucleic acid sequence that encodes OVA can be used as (c). Preferably such sequence is codon optimized for the host cell.

Illustrative transcriptional terminator elements include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbhl), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI1, homoserine kinase (THR1), tpi, TPS1, triose phosphate isomerase (TPI1), XRP2, YPT1, and any combination thereof.

Illustrative selectable markers (f) may include, but are not limited to: an antibiotic resistance gene (e.g. zeocin, ampicillin, blasticidin, kanamycin, nourseothricin, chloroamphenicol, tetracycline, triclosan, ganciclovir, and any combination thereof), an auxotrophic marker (e.g. ade1, arg4, his4, ura3, met2, and any combination thereof).

In one example, a vector for expression in Pichia sp. can include an AOX1 promoter operably linked to a signal peptide (alpha mating factor) that is fused in frame with a nucleic acid sequence encoding OVA, and a terminator element (AOX1 terminator) immediately downstream of the nucleic acid sequence encoding OVA.

In another example, a vector comprising a DAS1 promoter is operably linked to a signal peptide (alpha mating factor) that is fused in frame with a nucleic acid sequence encoding OVA and a terminator element (AOX1 terminator) immediately downstream of OVA.

In some embodiments, the recombinant protein (rOVA) described herein may be secreted from the one or more host cells. In some embodiments, rOVA protein is secreted from the host cell. The secreted rOVA may be isolated and purified by methods such as centrifugation, fractionation, filtration, ion exchange chromatography, affinity purification and other methods for separating protein from cells, liquid and solid media components and other cellular products and byproducts. In some embodiments, rOVA is produced in a Pichia Sp. and secreted from the host cells into the culture media. The secreted rOVA is then separated from other media components for further use.

In some embodiments, the rOVA mixture described herein may be secreted from the one or more host cells. In some embodiments, the rOVA mixture is secreted from the host cell. The secreted rOVA mixture may be isolated and purified by methods such as centrifugation, fractionation, filtration, ion exchange chromatography, affinity purification and other methods for separating protein from cells, liquid and solid media components and other cellular products and byproducts. In some embodiments, the rOVA mixture is produced in a Pichia Sp. and secreted from the host cells into the culture media. The secreted rOVA mixture is then separated from other media components for further use.

The present disclosure contemplates modifying glycosylation of the recombinant OVA to alter or enhance one or more functional characteristics of the protein and/or its production. In some embodiments, the change in rOVA glycosylation can be due to the host cell glycosylating the rOVA. In some embodiments, rOVA has a glycosylation pattern that is not identical to a native ovalbumin (nOVA), such as a nOVA from chicken egg. In some embodiments, rOVA is treated with a deglycosylating enzyme before it is used as an ingredient in an rOVA composition, or when rOVA is present in a composition. In some embodiments, the glycosylation of rOVA is modified or removed by expressing one or more enzymes in a host cell and exposing rOVA to the one or more enzymes. In some embodiments, rOVA and the one or more enzymes for modification or removal of glycosylation are co-expressed in the same host cell.

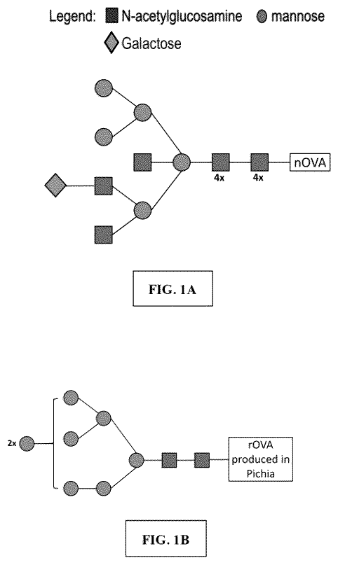

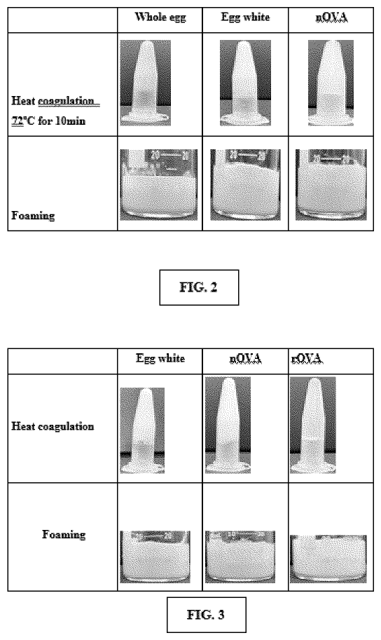

Native ovalbumin (nOVA), such as isolated from a chicken or another avian egg, has a highly complex branched form of glycosylation. The glycosylation pattern comprises N-linked glycan structures such as N-acetylglucosamine units, galactose and N-linked mannose units. See, e.g., FIG. 1A. In some cases, the rOVA for use in a herein disclosed consumable composition and produced using the methods described herein has a glycosylation pattern which is different from the glycosylation pattern of nOVA. For example, when rOVA is produced in a Pichia sp., the protein may be glycosylated differently from the nOVA and lack galactose units in the N-linked glycosylation. FIG. 1B illustrates the glycosylation patterns of rOVA produced by P. pastoris, showing a complex branched glycosylation pattern. In some embodiments of the compositions and methods disclosed herein, rOVA is treated such that the glycosylation pattern is modified from that of nOVA and also modified as compared to rOVA produced by a Pichia sp. without such treatment. In some cases, the rOVA lacks glycosylation.

The molecular weight or rOVA may be different as compared to nOVA. The molecular weight of the protein may be less than the molecular weight of nOVA, or less than rOVA produced by the host cell where the glycosylation of rOVA is not modified. In embodiments, the molecular weight of an rOVA may be between 40 kDa and 55 kDa. In some cases, an rOVA with modified glycosylation has a different molecular weight, such as compared to a native OVA (as produced by an avian host species) or as compared to a host cell that glycosylates the rOVA, such as where the rOVA includes N-linked mannosylation. In some cases, the molecular weight of rOVA is greater than the molecular weight of the rOVA that is completely devoid of post-translational modifications, or an rOVA that lacks all forms of N-linked glycosylation.

### Definitions

The terminology used herein is for the purpose of describing particular cases only and is not intended to be limiting.

As used herein, the singular forms “a” “an” and “the” are intended to include the plural forms as well, unless the context clearly indicates otherwise.

The terms “including”, “includes”, “having”, “has”, “with”, or variants thereof are used in either the detailed description and/or the claims, such terms are intended to be inclusive in a manner similar to the term “comprising”.

Ranges can be expressed herein as from “about” or “approximately” one particular value, and/or to “about” or “approximately” another particular value. When such a range is expressed, another case includes from the one particular value and/or to the other particular value. Similarly, when values are expressed as approximations, by use of the antecedent “about” or “approximately”, it will be understood that the particular value forms another case. It will be further understood that the endpoints of each of the ranges are significant both in relation to the other endpoint, and independently of the other endpoint. The term “about” or “approximately” as used herein refers to a range that is 15% plus or minus from a stated numerical value within the context of the particular usage. For example, about 10 would include a range from 8.5 to 11.5. The term “about” or “approximately” also accounts for typical error or imprecision in measurement of values.

An rOVA mixture disclosed herein comprises, consists essentially of, or consists of clipped forms of rOVA.

Any aspect or embodiment described herein can be combined with any other aspect or embodiment as disclosed herein.

### EXAMPLES

### Example 1: Preparation of Recombinant Ovalbumin

A Gallus gallus OVA coding sequence was fused in-frame with the alpha mating factor signal sequence downstream of the promoter sequence (SEQ ID NO:1). A promoter was placed upstream of the signal sequence OVA coding sequence and a transcriptional terminator was placed downstream of the OVA sequence. The expression construct was placed into a Kpas-URA 3 vector.

The expression constructs were transformed into Pichia pastoris. Successful integration was confirmed by genomic sequencing.

Fermentation: Recombinant OVA was produced in a bioreactor at ambient conditions. A seed train for the fermentation process begins with the inoculation of shake flasks with liquid growth broth using 2 ml cryovials of Pichia pastoris which are stored at −80° C. and thawed at room temperature prior to inoculation.

The inoculated shake flasks were kept in a shaker at 30° C. for 24 hours, after which the grown Pichia pastoris was transferred to a production scale reactor.

The culture was grown at 30° C., at a set pH and dissolved oxygen (DO). The culture was fed with a carbon source. At the end of the fermentation, the target OVA protein was harvested from the supernatant.

Cell debris was removed, protein was purified and lyophilized to a dry powder. The OVA produced was used in the examples described below.

### Example 2: Comparison of Foam Capacity and Foam Stability

This example evaluated the foam capacity/stability and coagulation properties of rOVA and compared it to fresh whole egg, egg white and nOVA.

Materials: store-bought egg, nOVA (Bioceutica), rOVA.

Method: A stock solution of OVA (nOVA, or rOVA) was made by mixing 0.7 g OVA in 9.3 g distilled water (total volume 10 ml). Cream of tartar was used (see Table 2 below) to adjust pH. Foam was made using a Dremel at speed 3. The time of whisking was recorded. Gel was made by heating 1 ml of sample at 72° C. for 10 min using a heat block.

TABLE 2
pH adjustments to rOVA, nOVA and egg white compositions
pH adjustment

| | | | Amount of | |
|---|---|---|---|---|
| | | | cream of | pH after |
| | | | tartar added | adding cream |
| | Initial pH | Temperature | (g) | of tartar |
| rOVA solution | 3.86 | 21 | 0 | 3.86 |
| nOVA solution | 5.45 | 20.7 | 0.1 | 4.01 |
| Fresh egg white | 8.57 | 20 | 2 | 4.64 |

Results of the foam capacity and stability are shown in the Table 3 below. In this set, pH was not adjusted.

$${}^{⋆} F oam capacity \% = [ Initial liquid Vol . ( ml ) / Foam Vol . ( ml ) ] ⋆ 100$$

$${}^{⋆} {}^{⋆} Foam stability \% = [ ( Initial liquid Vol . ( ml ) - Liquid drainage Vol . at 30 min ( ml ) ) / Initial liquid Vol . ( ml ) ] ⋆ 100$$

TABLE 3
Results of foam capacity and stability

| | Whole egg | Egg white | nOVA |
|---|---|---|---|
| *Foam | 210 ± 14.1 a | 300 ± 0 b | 338.5 ± 2.2 c |
| capacity % | | | |
| **Foam | 56 ± 2.8 b | 71 ± 1.4 a | 59.3 ± 0.92 b |
| Stability % | | | |
| time of whisking | >120 | 80 | 19 |
| (second) | | | |
| pH as is | 7.6 | 9.1 | 5.9 |

Conclusion: nOVA at pH 6 indicated the highest foam capacity compared to the egg white; however, its foam stability was lower than the egg white. Results are presented in FIG. 2.

The experiment was repeated using cream of tartar to adjust the pH.

TABLE 4
Results of foam capacity and stability after
pH adjustment using cream of tartar

| | Egg white | nOVA | rOVA |
|---|---|---|---|
| Foam capacity % | 316.3 ± 5.3 b | 457.9 ± 31.2 a | 367.9 ± 2.9 b |
| Foam Stability % | 83.6 ± 6.2 a | 65.1 ± 1.3 b | 60.5 ± 0.7 b |
| time of whisking | 64 | 19 | 32 |
| (second) | | | |
| Initial pH (as is) | 8.57 | 5.45 | 3.86 |
| Final pH (after | 4.65 | 4.01 | 3.86 |
| adjusting with | | | |
| cream of tartar) | | | |

Conclusion: The foam capacity of nOVA after reducing pH was still higher than egg white. The foam capacity of rOVA was higher in value compared to that of fresh egg white. The whisking time for rOVA was half that required for fresh egg white. Results are shown in FIG. 3

### Example 3: Preparation of Recombinant Chicken Ovalbumin Expression Strain

Expression Constructs Seven expression cassettes were created for expression of Gallus gallus OVA (SEQ ID NO: 2) in Pichia pastoris.

TABLE 5
Expression Cassettes of Interest

| Strain | Cassette | Promoter | Terminator |
|---|---|---|---|
| Chicken | GgOVA- | K phaffii AOX1 | K phaffii AOX1 |
| OVA | A1 | promoter | transcriptional terminator |
| Chicken | GgOVA- | K phaffii AOX1 | K phaffii AOX1 |
| OVA | A2 | promoter | transcriptional terminator |
| Chicken | GgOVA- | K phaffii AOX1 | K phaffii AOX1 |
| OVA | A3 | promoter | transcriptional terminator |
| Chicken | GgOVA- | K pastoris DAS | K phaffii AOX1 |
| OVA | D1 | promoter | transcriptional terminator |
| Chicken | GgOVA- | K pastoris FLD1 | K phaffii AOX1 |
| OVA | F2 | promoter | transcriptional terminator |
| Chicken | GgOVA- | K pastoris FLD1 | K phaffii AOX1 |
| OVA | F3 | promoter | transcriptional terminator |
| Chicken | HF-1 | K phaffii PEX11 | K phaffii AOX1 |
| OVA | | promoter | transcriptional terminator |

The first three cassettes were made to express a chicken OVA that comprises the amino acid sequence of chicken OVA (SEQ ID NO:2) fused in-frame with a nucleic acid encoding a secretion signal sequence; the expressed fusion protein has the amino acid sequence of (SEQ ID NO: 1). In each of the three cassettes, the Alcohol oxidase 1 (AOX1) promoter was placed upstream of the secretion signal sequence and a K phaffii AOX1 transcriptional terminator was placed downstream of the OVA-encoding sequence. These cassettes were labeled GgOVA-A1, GgOVA-A2, and GgOVA-A3 and combined into a first plasmid.

The fourth cassette included a chicken OVA coding sequence (which encodes SEQ ID NO: 2) fused in-frame with a nucleic acid encoding a secretion signal sequence (thereby encoding SEQ ID NO: 1) but with a dihydroxyacetone synthase (DAS2) promoter placed upstream of the secretion signal sequence and a K phaffii AOX1 transcriptional terminator placed downstream of the OVA-encoding sequence. This construct was labeled GgOVA-D1.

The fifth and sixth cassettes included the chicken OVA coding sequence (which encodes SEQ ID NO: 2) fused in-frame with a nucleic acid encoding a secretion signal sequence (thereby encoding SEQ ID NO: 1) but with a formaldehyde dehydrogenase (FLD) promote placed upstream of the secretion signal sequence and a K phaffii AOX1 transcriptional terminator placed downstream of the OVA-encoding sequence. These cassettes were labeled GgOVA-F1 and GgOVA-F2 and were combined with GgOVA-D1 in a second plasmid.

The seventh cassette included the peroxisome biogenesis (PEX11) promoter placed upstream of a Helper factor protein HAC1 coding sequence and a K. phaffii AOX1 transcriptional terminator placed downstream of the Helper factor sequence. This cassette was labeled HF-1 and was transformed into a third plasmid.

The three plasmids were transformed stepwise into a background strain of Pichia pastoris. Genomic sequencing confirmed integration of the expression constructs and copy number of each construct is shown in Table 6 below.

TABLE 6
Strain Genomic Composition

| | | Copies |
|---|---|---|
| Strain | Cassette | integrated |
| Chicken OVA | GgOVA-A1 | 1 |
| | GgOVA-A2 | 1 |
| | GgOVA-A3 | 1 |
| | GgOVA-D1 | 2 |
| | GgOVA-F2 | 2 |
| | GgOVA-F3 | 2 |
| | HF-1 | 8 |

### Example 4: Preparation of Recombinant Ovalbumin Expression Strains for Duck and Ostrich

Expression Constructs: one cassette for expression of Anas platyrhynchos (duck) OVA and one cassette for expression of Struthio camelus (ostrich) OVA were created for expression in Pichia pastoris.

TABLE 7
Expression cassettes of interest

| Strain | Cassette | Promoter | ORF | Terminator |
|---|---|---|---|---|
| Duck | ApdOVA | K phaffii AOX1 | Duck | K phaffii AOX1 |
| OVA | | promoter | OVA | transcriptional terminator |
| Ostrich | ScOVA | K phaffii AOX1 | Ostrich | K phaffii AOX1 |
| OVA | | promoter | OVA | transcriptional terminator |

One expression cassette was created for the expression of ostrich OVA. A nucleic acid encoding Struthio camelus OVA (SEQ ID NO: 71) was fused in-frame with a nucleic acid encoding a secretion signal sequence (thereby encoding SEQ ID NO: 72). The ostrich construct included the Alcohol oxidase 1 (AOX1) promoter placed upstream of the secretion signal sequence and a K phaffii AOX1 transcriptional terminator was placed downstream of the OVA sequence. This expression cassette called ScOVA was transformed into Pichia pastoris. Successful integration of four copies of the ostrich OVA construct was confirmed by genomic sequencing. See Table 15.

One expression cassette was created for the expression of duck OVA. A nucleic acid encoding Anas platyrhynchos OVA (SEQ ID NO: 73) was fused in-frame with a nucleic acid encoding a secretion signal sequence (thereby encoding SEQ ID NO: 74). The duck cassette included the Alcohol oxidase 1 (AOX1) promoter placed upstream of the secretion signal sequence and a K phaffii AOX1 transcriptional terminator was placed downstream of the OVA sequence. This expression cassette called ApdOVA was transformed into Pichia pastoris. Successful integration of two copies of the duck OVA construct was confirmed by genomic sequencing. See, Table 8.

TABLE 8
Strain genomic composition

| Strain | Cassette | Copies integrated |
|---|---|---|
| Duck OVA | ApdOVA | 2 |
| Ostrich OVA | ScOVA | 4 |

### Example 5: Fermentation and Production of rOVA

Fermentation: Strains for fermenting recombinant OVA (rOVA) were each cultured in a bioreactor at ambient conditions. A seed train for the fermentation process began with the inoculation of shake flasks with liquid growth broth. The inoculated shake flasks were kept in a shaker after which the grown P. pastoris was transferred to a production-scale reactor.

To expand production, a seed vial of rOVA P. pastoris seed strain was removed from cryo-storage and thawed to room temperature. Contents of the thawed seed vials were used to inoculate liquid seed culture media in baffled flasks which were grown at 30° C. in shaking incubators. These seed flasks were then transferred and grown in a series of larger and larger seed fermenters (number to vary depending on scale) containing a basal salt media, trace metals, and glucose. Temperature in the seed reactors was controlled at 30° C., pH at 5, and dissolved oxygen (DO) at 30%. pH was maintained by feeding ammonia hydroxide, which also acted as a nitrogen source. Once sufficient cell mass was reached, the grown rOVA P. pastoris was inoculated into a production-scale reactor containing basal salt media, trace metals, and glucose.

Like in the seed tanks, the culture was also controlled at 30° C., pH5 and 30% DO throughout the process. pH was again maintained by feeding ammonia hydroxide. During the initial batch glucose phase, the culture was left to consume all glucose and subsequently-produced ethanol. Once the target cell density was achieved and glucose and ethanol concentrations were confirmed to be zero, the glucose fed-batch growth phase was initiated. In this phase, glucose was fed until the culture reached a target cell density. Glucose was fed at a limiting rate to prevent ethanol from building up in the presence of non-zero glucose concentrations. In the final induction phase, the culture was co-fed glucose and methanol which induced it to produce rOVA via the pAOX promoters. Glucose was fed at an amount to produce a desired growth rate, while methanol was fed to maintain the methanol concentration at 1% to ensure that expression was consistently induced. Regular samples were taken throughout the fermentation process for analyses of specific process parameters (e.g., cell density, glucose/methanol concentrations, product titer, and quality). After a designated amount of fermentation time, secreted rOVA was collected and transferred for downstream processing.

The fermentation broth containing the secreted rOVA was subjected to centrifugation at 12,000 rpm. The supernatant was clarified using microfiltration. To concentrate the protein and remove excess water, ultrafiltration at room temperature was used. An appropriately sized filter was used to retain the target rOVA while the compounds, salts, and water smaller than rOVA passed through the filter. To reduce the final salt content and conductivity in preparation for chromatography, the concentrated rOVA retentate was dialyzed at pH 3.5 until the final conductivity of the material was 1.7 mS/cm. The bulk of the purification was done using cation exchange chromatography at pH 3.5. Citrate buffer containing a high salt concentration of sodium chloride was used to elute the bound rOVA from the resin. To remove the excess salts, the eluant was finally dialyzed to make a final protein solution containing about 5-10% protein and 85-95% water. The final solution was sterilized by passing it through a 0.2 um bioburden filter. The water was evaporated using a spray dryer/lyophilizer at appropriate temperatures to produce a final powder containing about 80% protein.

### Example 8: Preparation of Solubilized rOVA

In this example, hydrophobic recombinant chicken rOVA was solubilized and passed through a 0.2 μm filter.

Recombinant rOVA was purified through ion exchange chromatography at pH 3.5 and was found to be insoluble. Sodium hydroxide was added to the solution to change the pH to 12.5 and solubilize the rOVA. The rOVA solution at pH 12.5 was passed through a 0.2p m filter. Following filtration, the pH was returned to 6.5 using hydrochloric acid and the rOVA was spray dried or lyophilized. This dried chicken rOVA was then used in the Examples below.

### Example 6: Glycosylation of Gallus gallus rOVA

In this example, Pichia-secreted rOVA was analyzed for glycosylation patterns.

Native ovalbumin (nOVA) has two potential N-linked glycosylation sites (FIG. 1A). A single site of glycosylation at Asn-292 is found in the egg white. MALDI-TOF analysis has shown that the typical glycans on native OVA are organized as (Man)5(GlcNAc)5(Gal)1 (FIG. 1A) (Harvey et al., 2000). Analysis of glycans on rOVA showed a typical glycosylation pattern shown in (FIG. 1B).

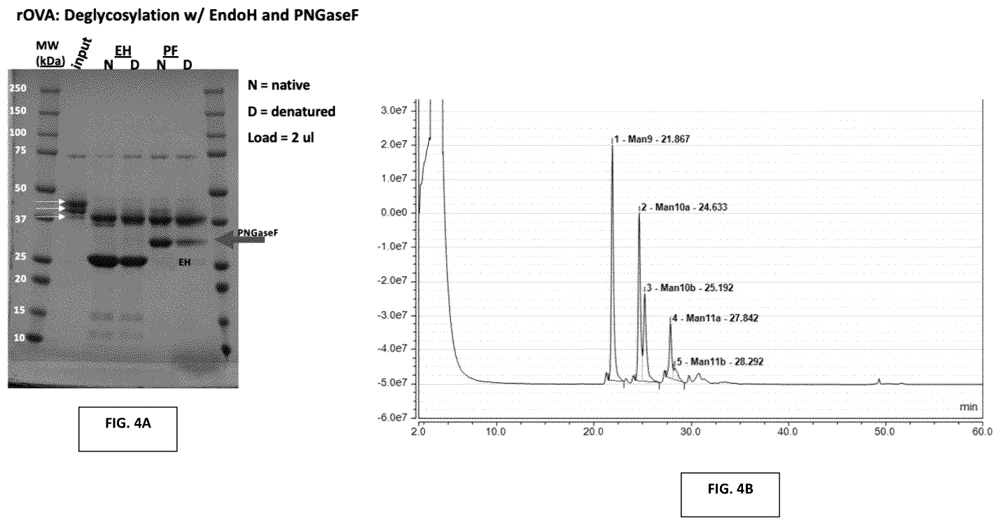

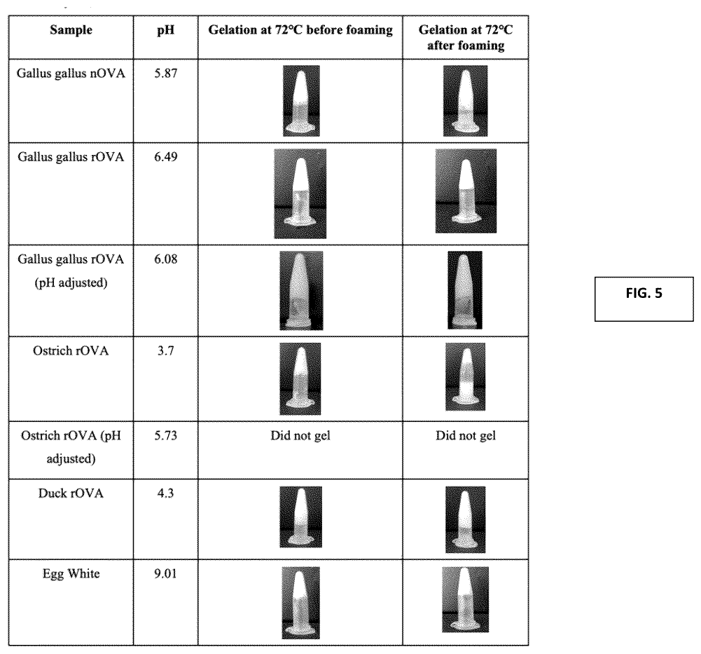

Pichia secreted chicken rOVA from the above Example was analyzed by gel electrophoresis migration and observed in three distinct forms (three white arrows pointing to rOVA in the “Input” lane below a) glycosylation-free, b) mono-glycosylated and c) di-glycosylated. Both the mono- and di-glycosylated glycosyl chains were cleaved from the mature rOVA protein using either of the endoglycanases EndoH or PNGaseF. Both the “denatured” or “native” deglycosylation protocols were used (as described in the NEB catalog). The green arrow indicates exogenous EndoH and the purple arrow indicates exogenous PNGaseF added to the in vitro reactions (FIG. 4A).

Pichia secreted chicken rOVA was subjected to standard analysis using Mass spectrometry. It was found to have five versions of N-linked Glycans (ManGlcNAc): high-mannose glycans of Man9 (˜40%), Man10 (˜47%) or Man11 (˜13%) type of N-glycan structures (FIG. 4B).

### Example 7: Comparison of Foaming Functionalities of Various Species rOVA

In this example, chicken rOVA, duck rOVA and ostrich rOVA were evaluated for properties of foaming ability and foam retention.

rOVA from ostrich and duck were produced, purified and lyophilized using methods similar to those set forth in Example 5 to 7. The ostrich rOVA and duck rOVA remained close to the acidic pH used for purification. Chicken rOVA was produced as set forth in Example 5 and solubilized at pH 12 before removing bioburden and returned to pH 6 before drying as set forth in Example 7.

Lyophilized rOVA samples were blended into distilled water. Clarity and solubility of the rOVA solutions were then assessed visually. All samples were compared to chicken nOVA and chicken rOVA.

Eleven mL of solution (7% w/v of protein) was created for each ostrich rOVA, chicken rOVA, and chicken nOVA. A 6 mL solution (7% w/v of protein) was created for duck rOVA due to limited availability of sample. Percent protein of the powders was used in the calculations to determine the amount necessary for a 7% solution. One mL of each solution was reserved before validation in a microtube for later use to test gelation. The samples were divided into 5 mL aliquots to be tested for foam capacity and stability.

Each 5 mL aliquot was pipetted into a beaker and whipped using the Dremel on speed 3. After a stiff foam was achieved, the foaming time was recorded as well as the initial volume of the foam. Foam capacity was determined by measuring the initial volume of foam following the whipping and comparing against the initial volume of 5 mL. Foam Capacity (%)=(volume of foam/initial volume)*100.

The drainage was measured in 10 minute increments for 30 minutes to gather data for foam stability. The drained volume after 30 minutes was compared to the initial liquid volume (5 mL). Foam Stability (%). (Initial volume−drained volume)/initial volume*100.

Chicken rOVA and ostrich rOVA were adjusted to pH 6 and tested again to ascertain effect of pH.

Chicken nOVA quickly formed stiff white foam. Ostrich rOVA foamed after 15 seconds. Duck rOVA foamed after 20 seconds.

TABLE 9
Foaming Parameters for rOVA in various species

| | | Foaming Time | Foam Capacity | Foam Stability |
|---|---|---|---|---|
| Sample | pH | (s) | (%) | (%) |
| Chicken nOVA | 5.87 | 16 | 415 | 66.5 |
| Chicken rOVA | 6.49 | 101 | 257 | 61 |
| Chicken rOVA | 6.08 | 21 | 417 | 66.7 |
| Chicken rOVA | 3.5 | 28 | 472 | 100 |
| Ostrich rOVA | 3.7 | 22 | 490 | 81.5 |
| Ostrich rOVA | 5.73 | 55 | 275 | 58 |
| (pH adjusted) | | | | |
| Duck rOVA | 4.3 | 26 | 400 | 70 |
| Egg White | 9.01 | 66.5 | 267.9 | 76.6 |

Table 9 shows the results for foaming time, foaming capacity, foam stability for chicken nOVA, at pH 5.87, chicken rOVA at pH 6.49 and pH 6.08, ostrich rOVA at pH 3.7 and pH 5.73, duck rOVA at pH 4.3 and egg white OVA at pH 9.0. Recombinant OVA from chicken, duck and ostrich generally had a similar or improved foaming capacity and foam stability as compared to egg white and these recombinant OVA proteins provided foaming capacity and foam stability between at least pH 3.5 and 6.5. Foam capacity and foam stability of rOVAs provide utility in compositions such as baked compositions.

### Example 8: Comparison of Gelation of Various rOVA Species

In this example, chicken, duck, and ostrich rOVA protein were evaluated for gelation properties. Gelation properties provide utility in applications such as cooked egg compositions.

One mL of each OVA solution was reserved for use to test gelation. After the Dremel procedure and foaming test in Example 2 was completed, another 1 mL sample was extracted from the drained liquid (containing the OVA) and pipetted into another microtube. Both the fractions collected, before and after foaming, were placed in a water bath and heated to 72° C. for 10 minutes. Samples were observed for gel formation.

FIG. 5 shows the results for gelation before and after foaming for chicken nOVA, at pH 5.87, chicken rOVA at pH 6.49 and pH 6.08, ostrich rOVA at PH 3.7 and pH 5.73, duck rOVA at pH 4.3 and egg white OVA at pH 9.0. Duck rOVA showed better gelation characteristics compared to chicken rOVA. Duck rOVA had gelation functionality close to that of natural egg white.

These data showed that the favorable properties disclosed above for the recombinant chicken OVA are also obtainable with recombinant OVAs from other species.

### Example 9: Comparison of Foaming rOVA Solutions

In this example, rOVA (chicken), solutions were compared to fresh egg white and evaluated for properties of foaming ability and foam retention.

Lyophilized samples were blended into aqueous solution (distilled water) at different concentrations and pHs. Clarity and solubility of the solutions was then assessed visually for foaming ability and foaming retention.

Protein solutions were created for each 4% rOVA, 7% rOVA, Fresh Egg White (12% protein), and 12% rOVA. Percent protein of the powders was used in the calculations to determine the amount necessary for each solution. 1 mL of each solution was reserved before validation in a microtube for later use to test gelation. The samples were divided into 5 mL aliquots to be tested for foam capacity and stability.

Each 5 mL aliquot was pipetted into a beaker and whipped using the Dremel on speed 3. After a stiff foam was achieved, the foaming time was recorded as well as the initial volume of the foam. Foam capacity was determined by measuring the initial volume of foam following the whipping and compare against the initial volume of 5 mL. Foam Capacity (%)=(volume of foam/initial volume)*100.

The drainage was measured in 10-minute increments for 30 minutes to gather data for foam stability. The drained volume after 30 minutes was compared to the initial liquid volume (5 mL). Foam Stability (%). (Initial volume−drained volume)/initial volume*100.

TABLE 10
Foaming functionality for chicken rOVA

| Protein | | Foaming | Foam Stability | Time Spent |
|---|---|---|---|---|
| Combination | pH | Capacity (%) | (%) | Foaming (s) |
| Fresh Egg White | 9.01 | 268 | 77 | 67 |
| (12% protein) | | | | |
| 4% OVA | 6.05 | 333 | 57 | 25 |
| 7% OVA | 6.03 | 333 | 66 | 19 |
| 12% OVA | 6.05 | 313 | 69 | 18 |

rOVA at 4%, 7% and 12% has greater foaming capacity, more foaming stability, and forms a foam more quickly than fresh egg white.

### Example 10: Foaming Functionality

In this example, the foaming functionality of rOVA was observed in an alcohol-based drink (e.g., such as a Whiskey Sour which includes a foaming agent).

Bourbon whisky, fresh lemon juice, simple syrup, and protein of interest were combined in a cocktail shaker and shaken for 15 seconds. Ice was added to the cocktail shaker and the mixture shaken for another 15 seconds. Shaken mixture was poured into a glass and observed.

Formulations: Control formulation included natural egg white. The negative formulation was prepared without any egg white.

TABLE 11
List of ingredients and the formulations

| Ingredient | Ounces | mL |
|---|---|---|
| Bourbon Whiskey | 2 | 59 |
| Fresh Lemon Juice | 0.75 | 22.125 |
| Simple syrup | 0.5 | 14.75 |
| Egg white | 0.5 | 14.75 |
| Total | 3.75 | 110.625 |

The proteins of interest were used to substitute the natural egg white protein and the following formulations were used:

TABLE 12
Protein formulation

| Ingredients | 7% rOVA | 12% rOVA |
|---|---|---|
| rOVA | 8.40 | 14.41 |
| Water | 91.60 | 85.59 |
| Total | 100 | 100 |

The pH of the rOVA solutions was adjusted to pH 6 (with 1M NaOH) to provide optimal foaming performance.

Original recipe used 0.5 oz egg white and the same proportion was used for recombinant protein testing. rOVA at 7% and 12% foamed well but no significant difference was observed between the two levels.

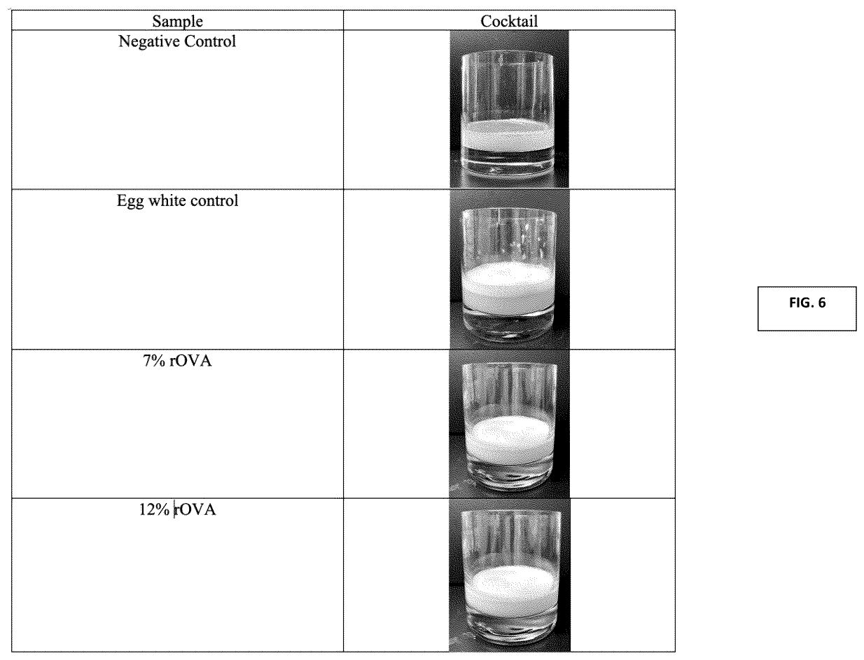

Photographs of craft cocktails prepared with the samples are shown in FIG. 6.

### Example 11: Effect of pH on Gelation Characteristics

The effects of different pH conditions on the gelation characteristics of rOVA compositions in comparison to fresh egg white was evaluated in this example.

TABLE 13
Materials:
Ingredients

DI water, 1N Hydrochloric acid, 1N Sodium hydroxide, 3N
Sodium hydroxide
Proteins of interests
rOVA (008USU_CW-86.1% protein content)
Egg white protein (Modernist pantry-85.71% protein content)

### Method:

1. 7% protein solution was prepared for both rOVA and egg white protein
2. Based on the native pH, the pH of the solution was adjusted to pH 3,4,5,6 with 1N HCl
3. pH was also adjusted to the alkaline spectrum of pH 7, 8, 9, 10, 11 and 12 with microliter amounts of 1N and 3N sodium hydroxide
4. All solutions were gelled at 85° C. for 5 min and then cooled at room temperature
5. All the gels/solutions were taken out and evaluated visually for gel characteristics

TABLE 14
Results: pH was recorded as follows before any pH adjustments:

| Sample | pH |
|---|---|
| 7% EWP | 6.98 |
| 7% rOVA | 6.82 |

Findings: Egg white protein exhibited gelling properties at all pH's while forming firm gels at pH 4-10. The solutions for both EWP and rOVA at pH 11 and pH 12 were clear liquids, however, only EWP gelled into clear gels, while rOVA remained in solution at pH 11 and 12. rOVA 7% solutions gelled at pH 6, 7, 8 and 9. Dramatic increase in viscosity was observed for rOVA solutions at pH 5 and lower. All EWP gels had a strong egg-like smell, while for rOVA, only solutions/gels for pH 9-12 had an egg-like smell. pH 3.5-8 for rOVA did not have any characteristic smell properties. EWP and rOVA both gelled at pH 6-9; however, EWP gels were stronger and firmer than rOVA gels. Overall, although EWP exhibited better gelling properties than rOVA over a broader pH spectrum, it came with the presence of a strong egg-like smell. rOVA provided gelling properties in the pH 6-8 range and provided sensory neutrality (e.g., no smell). At pH 8 and 9, rOVA provided clear firm gel which can have unique value proposition in embodiments requiring transparent visual appearance.

### Example 12: Preparation and Analyses of rOVA Mixture Containing Clipped rOVA

An rOVA mixture (e.g., an rOVA mixture comprising one or more clipped forms of the rOVA) was used as a protein source in a beverage application.

OVA typically forms a turbid solution at high concentrations and very sensitive to gelation with heat exposure. A beverage product that stays clear colorless at 12% solution and does not gel with heat exposure was successfully prepared using rOVA produced using the methods described in earlier examples. The prepared beverage product has high foaming characteristics with a stable foam similar to the egg white powders.

### Preparation:

The process was performed at the lab scale using a strain producing rOVA Supernatant comprising the rOVA was filtered. The filtered supernatant was then concentrated, diafiltered, modified its pH to 3.5, and passed through a cation exchange column. The expressed proteins were then eluted with sodium chloride in citrate buffer and subsequent purified using filtration unit and thoroughly diafiltered to remove the excess salts.

The purified protein concentrate was then heat treated to 60° C. under constant stirring for 12 min followed by rapid cooling. The final product was lyophilized for testing.

### Results:

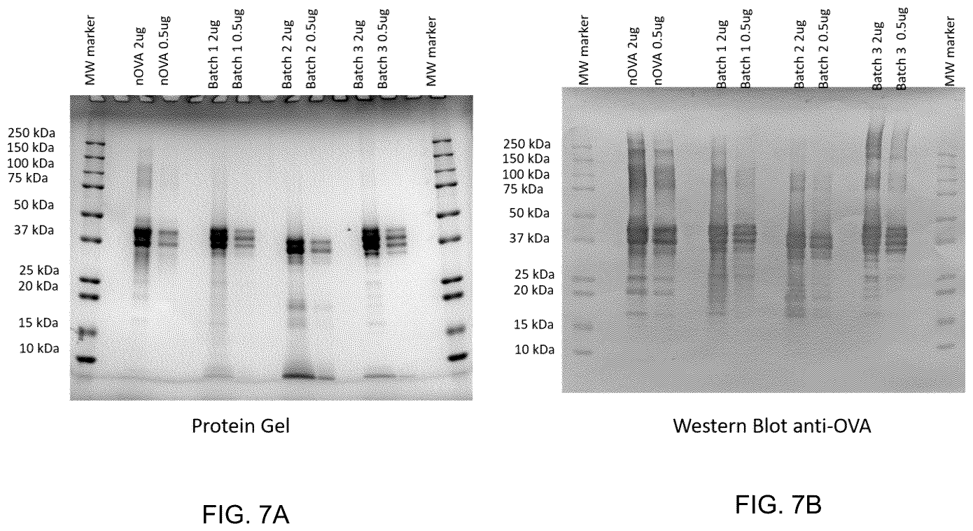

Various gel analyses indicated that there were two recombinant forms of the expressed rOVA observed in different batches of purified proteins. As shown in FIGS. 7A-7B, the protein gel and the Western Blot anti-OVA gel analyses of three different batches (batches #1 through 3) of the expressed rOVA indicated that the produced protein was a mixture containing at least one clipped form of the rOVA.

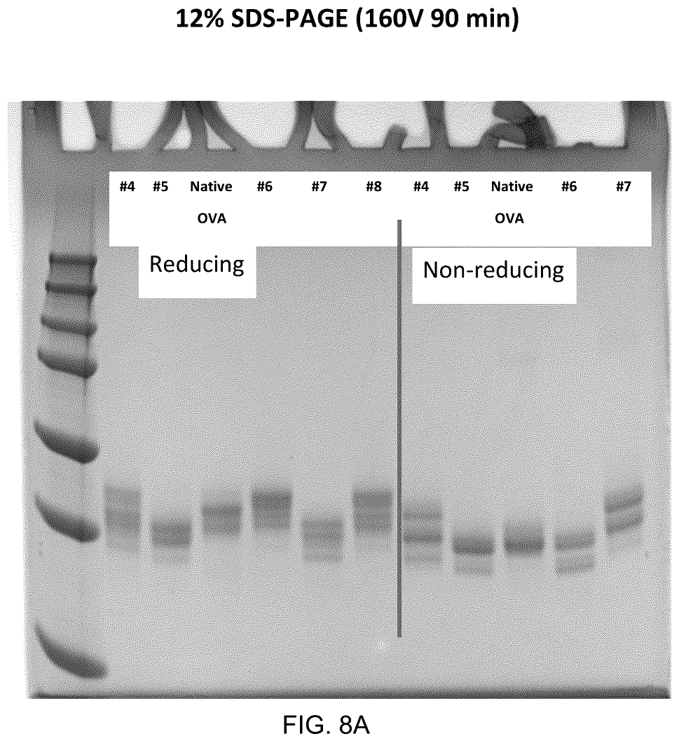

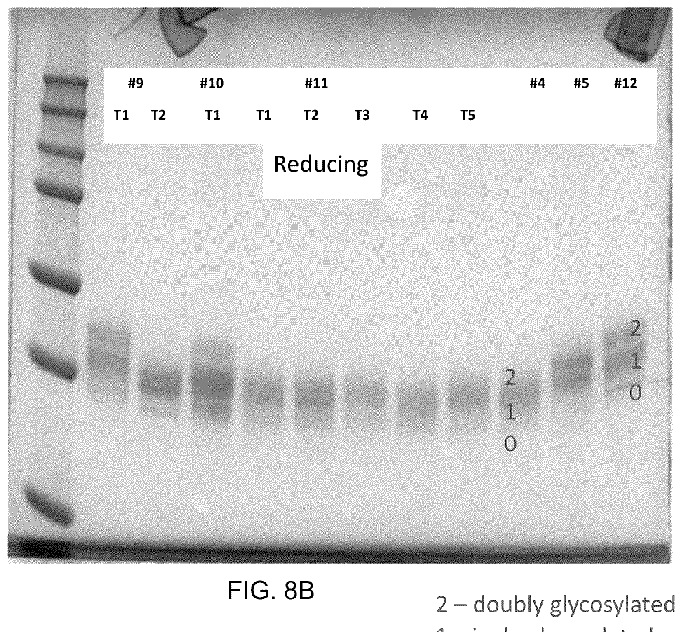

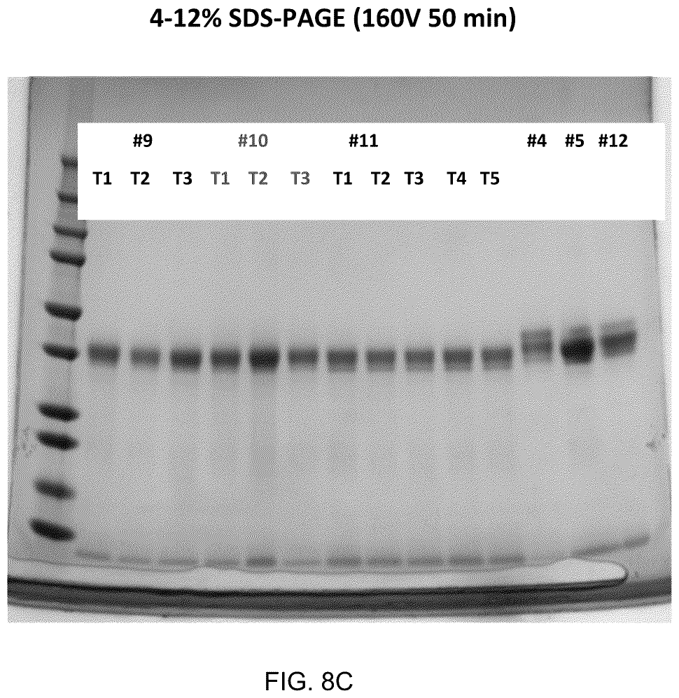

As shown in FIGS. 8A-8C, the SDS-PAGE analyses of additional various batches (batches #5 and #7 in FIG. 8A, and batches #5, #9, #10, and #11 in FIGS. 8B-8C) also indicated that produced protein was a mixture containing a clipped form of the OVA. In comparing batch #4 with other sample batches under denaturing conditions in FIGS. 8A-8B, a shift in the ˜40 kD protein bands and the appearance of a low molecular weight band (shown in the 4-12% SDS-PAGE gel) were observed. FIG. 8C shows that correlating band shift with additional fragments were observed on the 12%-SDS-PAGE gel, e.g., for batch #9 (Trial 1, Trial 2, and Trial 3), batch #10 (Trial 1, Trial 2, and Trial 3), batch #11 (Trial 1, Trial 2, and Trial 3), and batch #5.

The characteristics of the full length (non-clipped) and the clipped forms of the rOVA (50% clipped or 100% clipped) at higher concentrations, e.g., at 7% and 12% concentration (w/v) are summarized in Table 15.

TABLE 15
Characteristics of the full length rOVA

| | 12% Concentration | 7% Concentration |
|---|---|---|
| Foam Capacity | 538% ± 18 | 525% ± 35 |
| Foam Stability | 89% ± 2 | 60% ± 0 |
| Hardness | 1374.40 ± 351.52 | 328.05 ± 68.88 |
| Adhesiveness | 0.43 ± 0.45 | 1.05 ± 0.50 |
| Hardness | 1180.93 ± 292.61 | 113.50 ± 35.45 |
| Cohesiveness | 0.64 ± 0.04 | 0.11 ± 0.08 |
| Springiness | 4.26 ± 0.01 | 3.58 ± 0.04 |
| Chewiness | 37.16 ± 10.64 | 1.29 ± 1.20 |

However, when comparing the characteristics of the clipped form of the rOVA with the full length (not truncated/clipped), it was surprisingly found that the clipped (clipped) rOVA shows poor gelation characteristics. In addition, a 12% w/v solution of the clipped rOVA in water is a clear colorless solution. Precision fermentation-based proteins to produce products with clipped rOVA allows production of high concentration high foaming protein products.

### Example 13: Heat Treatment

The egg white proteins generally have a very narrow range of functionality with respect to gelation and foaming. However, with the controlled fermentation of recombinant OVA, rOVA was produced in multiple process conditions to generate material that could function as a high gelling product. Further, surprisingly this modulation of the properties was achieved by varying the process conditions, e.g., with or without heat treatment, and producing full length rOVA or clipped rOVA. The modulation of functionality was monitored with analytical tools around the identification of thermal denaturation and protein clipping using a standard 12% native gel.

### Experiments:

To test the properties of the protein mixture produced, protein solutions were prepared. The resulting protein solutions passed through multiple purification steps at low temperatures (10-15° C.). Subsequently, the resulting protein solutions were exposed to a heat treatment consisting of three steps rapid heating to 60° C., holding the product at 60° C. for 12 min and then cooling the product to 10-14° C. range. This heat-treated material was then spray dried with the air inlet temperature around 135° C. and with an exit temperature of around 65° C. Alternatively, a tangential flow filtration was used instead of heat treatment for the bioburden reduction with the least heat exposure.

Tests were performed with multiple cooling cycles leading to a variation in the heat load to the product. Finally, the products generated were tested in formulations such as pound cake and the data was generated around the variation of properties.

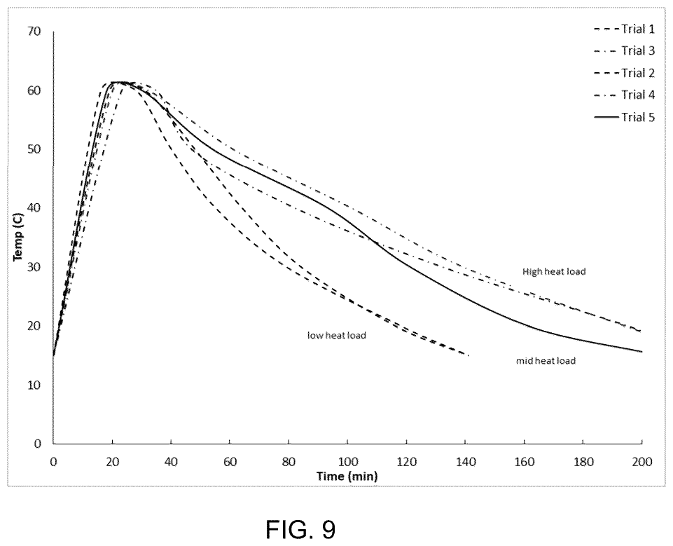

The heat profiles tested were shown in FIG. 9. The details of the process conditions are shown in Table 16 below.

TABLE 16
Summary of the heat treatment process conditions

| | Trial-1 | Trial-2 | Trial-3 | Trial-4 | Trial-5 |
|---|---|---|---|---|---|
| Pasteurization Feed volume (L) | 25 | 25 | 50 | 50 | 90 |
| Time taken for Heating up to (60° C.) min | 19 | 16 | 20 | 23 | 18 |
| Hold at 60 C. for 12 min | 12 | 12 | 12 | 12 | 12 |
| Time taken for chilling from (60° C. to 50° C.) min | 17 | 12 | 29 | 12 | 24 |
| Time taken for chilling from (50° C. to 40° C.) min | 16 | 15 | 40 | 35 | 39 |
| Time taken for chilling from (40° C. to 30° C.) min | 20 | 24 | 38 | 50 | 28 |
| Time taken for chilling from (30° C. to 20° C.) min | 32 | 39 | 55 | 63 | 40 |
| Time taken for chilling from (20° C. to 15° C.) min | 25 | 23 | 32 | 29 | 45 |
| Total time taken for chilling (min) | 110 | 113 | 194 | 189 | 176 |

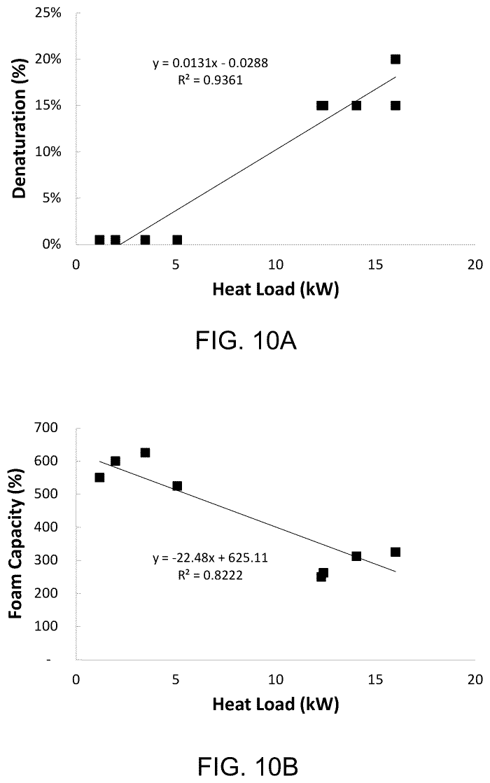

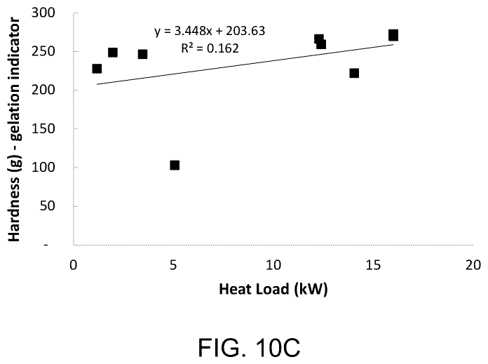

The analysis of the material clearly showed a variation in the product characteristics as the heat exposure went from least with tangential flow filtration to highest with the slow cooling step. As shown in FIGS. 10A-10C, a correction was observed between heat load and the characteristics of denaturation, foam capacity, and hardness (gelation indicator) of the rOVA mixture comprising clipped rOVA batch tested. For example, as shown in FIG. 10A, a positive correlation between the heat exposure in the process and denaturation as measured on the bench. FIG. 10B shows that the foam capacity of the products decreases significantly as we increase the heat load. Surprisingly, FIG. 10C shows that there is a slight positive correlation with heat exposure to the gelling characteristics of the product, because high heat load leading to denaturation of the product generally leads to reduction in the gelling ability of the protein. Thus, the foaming and gelation characteristics of the rOVA mixture can be modulated by controlling the heat treatment conditions (e.g., temperature, duration, and the cooling process).

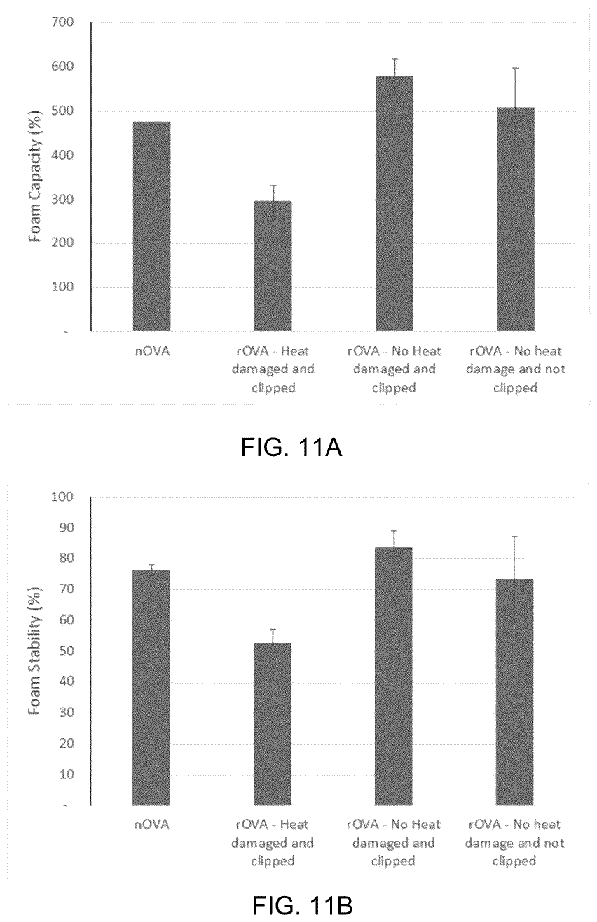

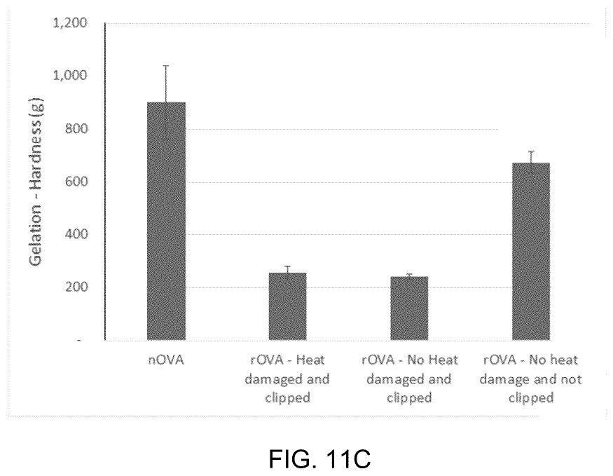

The products were also processed through basic parameter analysis on a 12% solution of the protein in deionized (DI) water for foaming and gelation shown in FIGS. 11A-11C.

FIGS. 11A-11C showed that the clipped rOVA without heat treatment exhibit the highest foam capacity and stability, but a reduced gelation (hardness) as compared to the full length rOVA without heat treatment. Also as shown FIGS. 11A-11C, the heat treatment decreases the product performance with respect to foam capacity and stability, and clipping phenomenon makes the heat damage worse. However, if heat damage is avoided, both the full length (unclipped) and clipped proteins can function as high foaming and clear colorless and tasteless protein solutions at high concentrations.

Two batches of products (batch #9 (sample 009) and batch #10 (sample 006)) were treated with different heat treatment conditions (Trials 1-5) to observe any deviations from the control and to study effect of different pasteurization conditions.

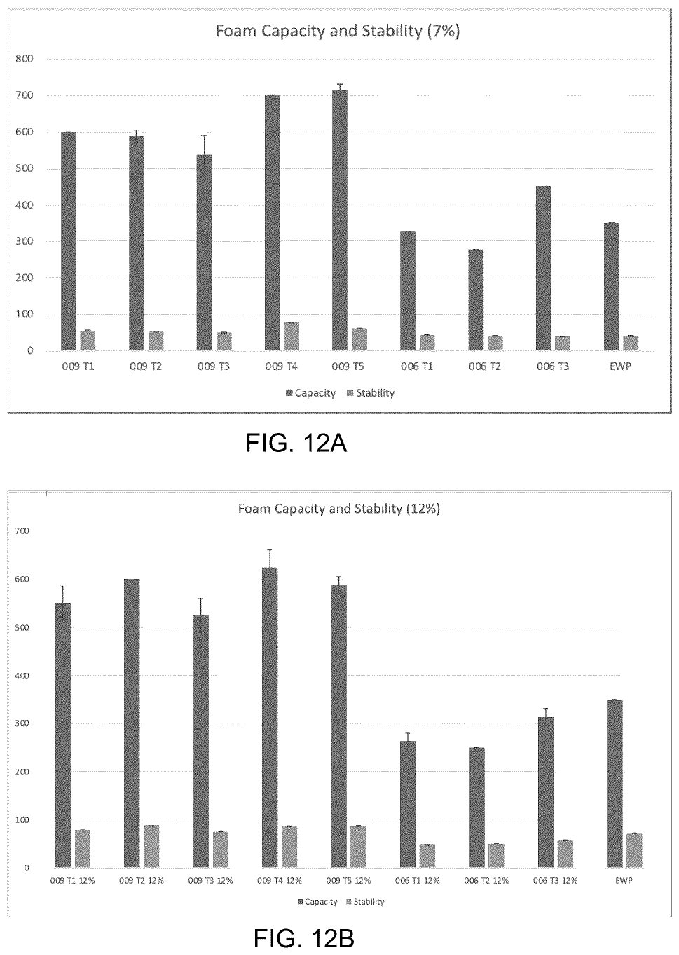

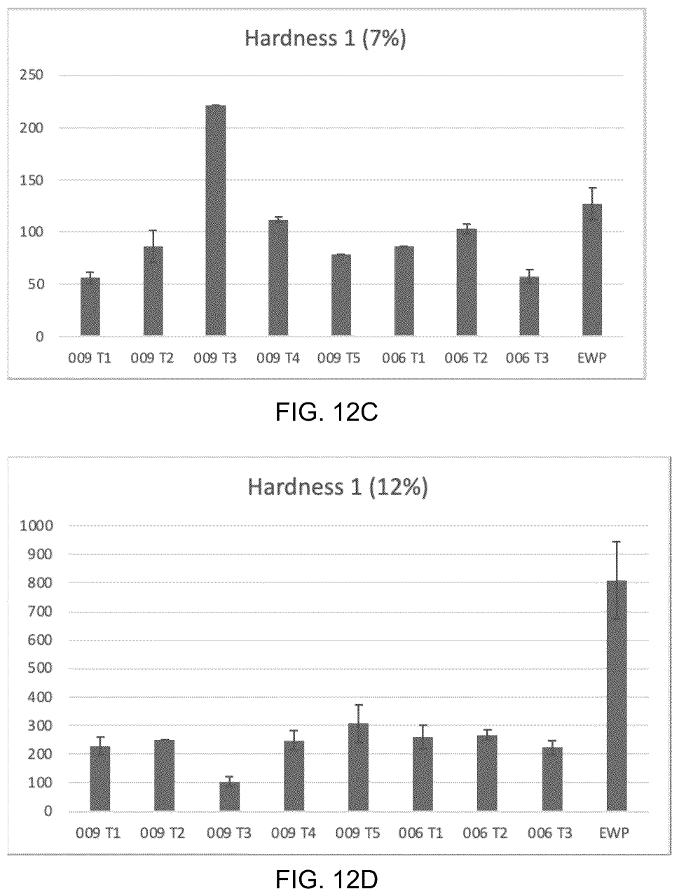

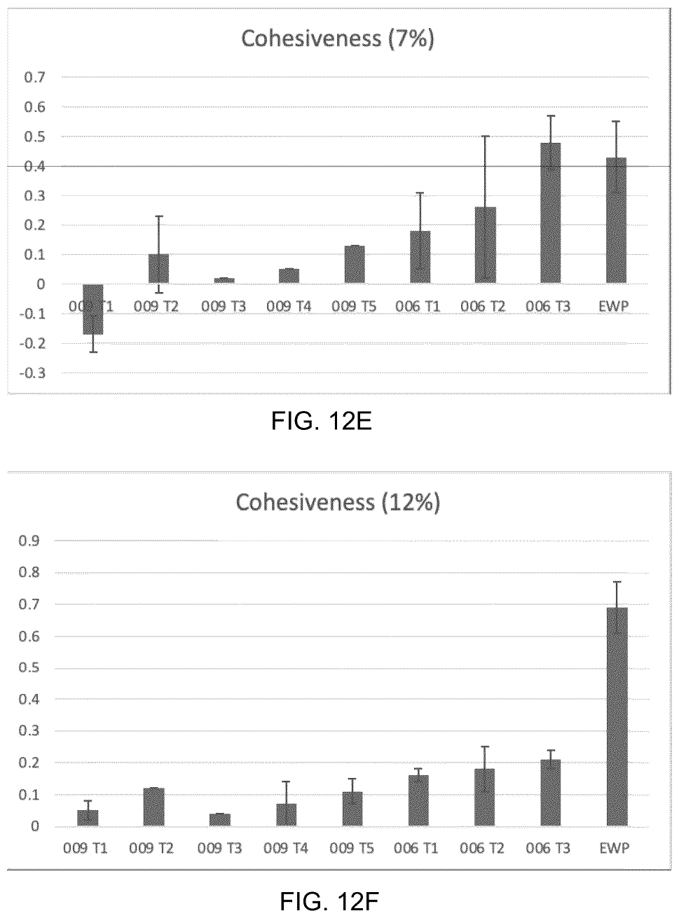

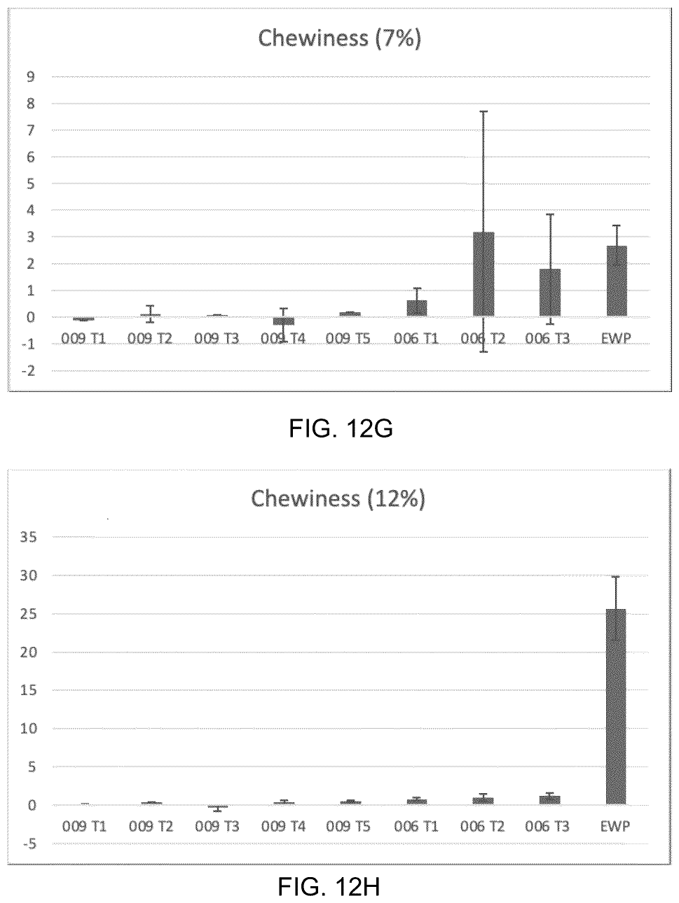

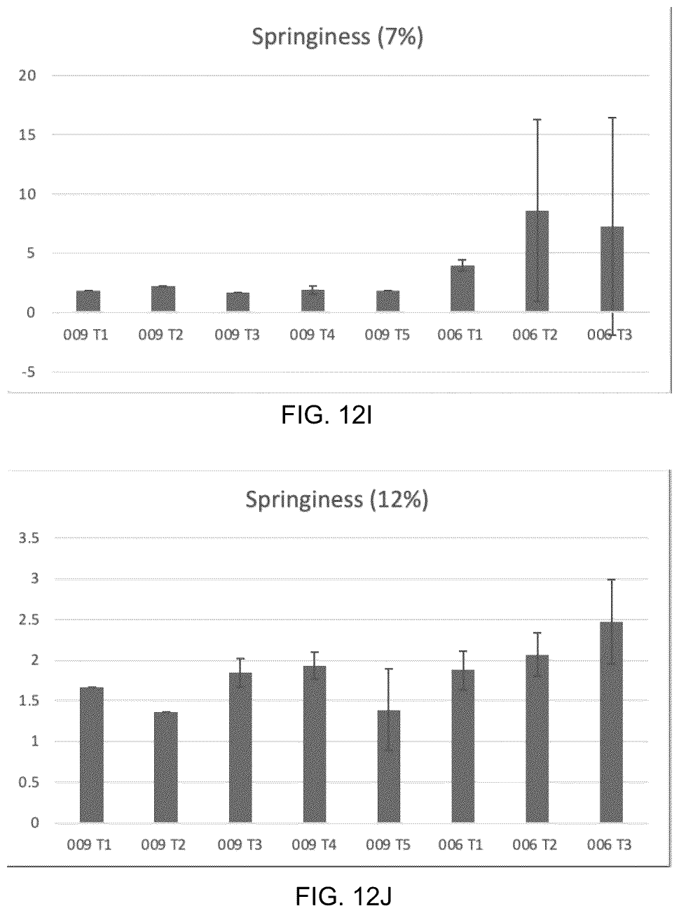

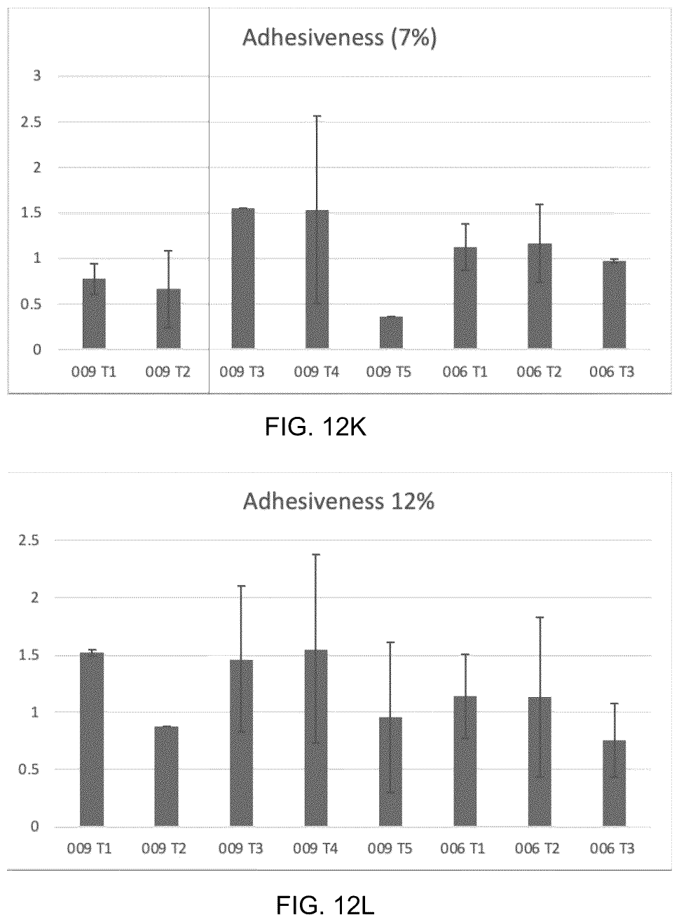

As shown in FIGS. 12A-12L, the foam capacity and stability, gelation (hardness), cohesiveness, chewiness, springiness, and adhesiveness were evaluated of batch #9 (sample 009) treated by heat conditions Trials 1-5, and batch #10 (sample 006) treated by heat conditions Trials 1-3. As shown in FIGS. 12A-12B, the foam capacity and stability of #9 (sample 009) and clearly outperformed batch #10 (sample 006) and egg white protein (EWP). From FIGS. 12C and 12D, it was also observed that the gelation characteristics of batch #10 (sample 006) samples was lower than egg white protein, but similar to the gelation characteristics of batch #10 (sample 006). The hardness of batch #10 (sample 006) treated with Trial 3 conditions was significantly lower than the other 4 treatment conditions and batch #10 (sample 006).

Both the batch #9 (sample 009) and batch #10 (sample 006) were also evaluated using the sensory analyses, the results of which are summarized in Table 17 below.

TABLE 17
Sensory analyses of batch #9 (sample 009)

| Sample | Appearance | Smell | Taste | Texture | Aftertaste |
|---|---|---|---|---|---|
| 009 T1 | white, very opaque, | mild to moderate | mild to moderate | strong pasty, very | very mild savory |
| | moderately crumbly, | chicken broth, | yeasty, mild savory/ | mild mouth | |
| | moderately smooth | mildly salty, very | brothy, cough | coating, moderate | |
| | surface. | mild sweet | syrup | brittleness, broke | |
| | | | | easily | |
| 009 T2 | very white, very | moderate chicken | mild popcorn, mild | strong pasty, | very mild savory |
| | opaque, very | broth, moderate | butter, very mild to | moderate bite, | |
| | smooth surface, | cooked meat, | mild savory | mild | |
| | moderately crumbly | very mild yeasty | brothy, very mild | mouth coating, | |
| | | | yeasty | mildly brittle | |
| 009 T3 | moderate to strong | moderate chicken | mild savory | moderately pasty, | No aftertaste |
| | white, very opaque, | broth | brothy, very mild | mild bite, very | |
| | strong smooth | | sweet | mild to mild | |
| | surface, mild to | | | mouth coating | |
| | moderately crumbly | | | | |
| 009 T4 | moderate to strong | mild chicken | mild savory | strong pasty, mild | No aftertaste |
| | white, very opaque, | broth, mild | brothy, mild butter | to moderate bite, | |
| | strong smooth | sweet, very mild | | mild mouth | |
| | surface, mild to | to mild cooked | | coating, | |
| | moderately crumbly | meat | | moderately brittle | |
| 009 T5 | strong white, very | moderate chicken | mild cough syrup, | very pasty, mild | very mild rubbery, |
| | opaque, moderately | broth | mild to moderate | mouth coating, | very mild cough |
| | smooth surface, | | savory brothy, | mildly brittle | syrup |
| | mild to moderately | | sulfur, very mild | | |
| | crumbly | | yeasty | | |

### Example 14: Production and Confirmation of Recombinant Full-Length Ovalbumin and Clipped Ovalbumin

Full-length and clipped forms of ovalbumin were produced as discussed in Example 12. Both full-length and clipped ovalbumin proteins were purified by column chromatography. Upon denaturation and SDS-PAGE, the purified intact ovalbumin provides a single band for full length protein while the purified clipped ovalbumin generates two bands consistent with the fragments present together in a complex. On isoelectric focusing and native-PAGE gels, clipped and intact ovalbumin both run as single species with similar migration patterns. The intact protein migrates slightly more than the clipped complex for both gel types. Western blot analysis confirmed that the clipped protein and fragment still had the expected OVA primary acid sequence. FIG. 13A illustrates a structure of ovalbumin protein. An illustrative clipping site—Ala352(P1)-Ser353(P1′) is highlighted. The green part of the structure is one continuous polypeptide backbone, and the clipped purple backbone structure is another continuous polypeptide backbone. The two backbones are still connected, as shown in FIG. 13A via non-covalent bonds. FIG. 13B illustrates a SDS-PAGE gel showing clipping in various batches of ovalbumin via migration shift and small fragment appearance at the bottom of the gel. OVA standards for intact and clipped forms were included as controls (lanes 13 and 14 respectively).

The 0% clipped (full-length) ovalbumin and 100% clipped ovalbumin protein powders were combined to produce a 50% full-length, 50% clipped ovalbumin protein powder. The three protein powders: 100% full-length, 50% full-length (50% clipped) and 100% clipped ovalbumin protein powders were tested against each other for egg-white like properties.

### Example 15: Rheology Results

Food materials fall on a spectrum of behaving fluid-like to solid-like, which determines a broad range of technical properties from cooking processing to mouth sensory experience. Viscoelasticity is used to describe materials that are intermediates of solids and fluids, having some combination of flow and elastic behaviors. Rheology provides a method to quantify viscoelasticity, allowing for a highly sensitive and applicable metric for classifying these materials.

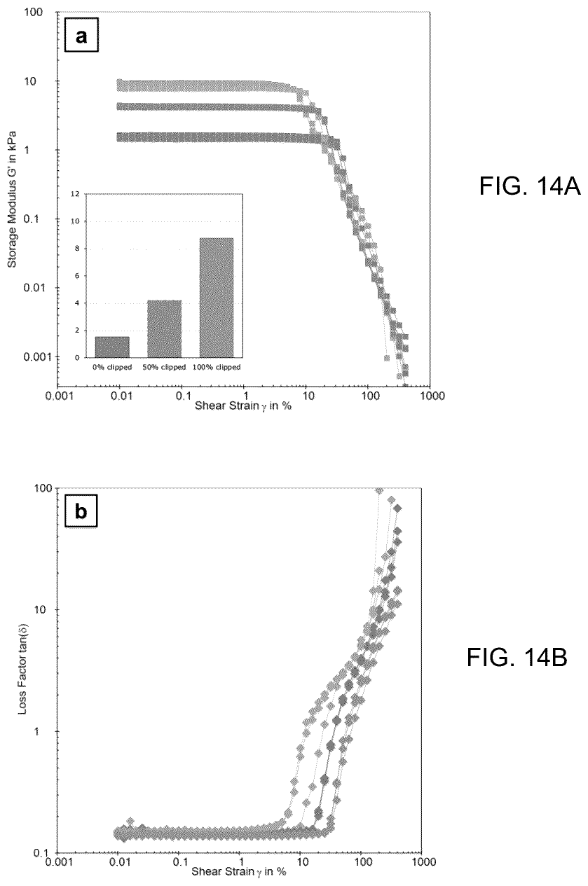

FIGS. 14A and 14B show data from an amplitude sweep of 0%, 50%, and 100% clipped protein dispersions in 20 mM phosphate buffer (pH 7). Amplitude sweeps provide information on various properties such as structural stability, level of fluidity or elasticity, rigidity (stiffness), and how easily a material spreads. The extent of the plateau region in FIG. 14A indicates the structural strength and stability, or how easily a material flows and spreads. 100% clipped material is more spreadable than 0% clipped material at higher shear strain values (above 2% on the x-axis). This spreadability difference is corroborated by the lower shear strain values at which the loss factor increases/spikes shown in FIG. 14B. The loss factor is a ratio of the fluid-like behavior to the solid-like behavior, so that the higher loss factor at lower shear strains indicates a more fluid-like and spreadable material.

The magnitude of the storage modulus provides the materials stiffness. Notably, at lower shear strain values, 100% clipped material is stiffer as seen by the higher magnitude of the storage modulus (G′) compared to the modulus of 50% and 0% clipped material (FIG. 14A, inlet). This results in a material that is rigid and resistant to flow during low amplitudes of displacement (0.01-2% on the x-axis).

These properties may indicate that clipped material behaves in a solid-like state when it is not being disturbed significantly, but with enough force will smoothly flow better than unclipped material. Overall having the properties of a stiff material that is also more spreadable.

### Example 16: Methods for Reducing Clipping

In some examples, a host cell producing heterologous ovalbumin may produce proteases that lead to clipping of ovalbumin during the fermentation process. Protease inhibitors may be used to modulate the amount of clipping in rOVA during fermentation. The protease inhibitors discussed herein are illustrative and can include several other examples. One of the prevalent clipped sites in ovalbumin include a serine site. Serine protease inhibitors may be used to modulate the amount of clipping in rOVA. Some illustrative serine protease inhibitors are provided in Table 18 below.

The amount of enzymes added to the fermentation medium may depend on the amount of clipping required. For instance, to create a protein mixture of OVA which includes a limited or no amount of clipped rOVA, a higher amount of protease inhibitors may be added. Alternatively, to produce a mixture of rOVA which primarily contains clipped OVA, only a small amount of protease inhibitors may be added.

TABLE 18
Illustrative serine proteases and their potential inhibitors

| Serine protease | Serine protease inhibitor |
|---|---|
| Thrombin | Protease-nexin-1 (PN-1), antithrombin III colligin, |
| | phosphatidylethanolamine-binding protein |
| Tissue plasminogen | Plasminogen activator inhibitor-1 (PAI-1), |
| activator (tPA) | neuroserpin, PN-1 |
| Plasmin | α2-antiplasmin, PN-1 |
| Trypsin | PN-1, α1-antitrypsin |
| Neuropsin | Serine protease inhibitor 3, murinoglobin I |

### Example 17: Macarons

Macarons comprise a pair of shells, which are made from the combination of an almond paste and a meringue, and a vegan butter cream sandwiched between the two shells. rOVA and rcOVA were prepared as provided in earlier examples. The three protein powders: 100% full-length, 50% full-length (50% clipped) and 100% clipped ovalbumin protein powders were tested against each other for egg-white like properties.

TABLE 19
Macaron Ingredients (in grams)

| Ingredient | Amount |
|---|---|
| Almond Paste Ingredients | |
| Almond Flour Super Fine | 135.3 |
| Powdered Sugar 10× | 135.3 |
| Ovalbumin Liquid | 53.3 |
| Meringue Ingredients: | |
| Ovalbumin Liquid | 58.6 |
| Cream of Tartar | 0.1 |
| Granulated Sugar | 150.5 |
| Water | 60.2 |
| Vanilla Bean Paste | 6.7 |
| Total for shell | 600 |
| Cream Ingredients | |
| Plant Based Butter | 90.7 |
| Powdered Sugar 10× | 193.8 |
| Coconut Milk Unsweetened | 9.3 |
| Salt | 0.8 |
| Vanilla Bean Paste | 5.4 |
| Total for cream | 300 |

Shells: Granulated sugar and water were mixed and heated to 120° C. to form a sugar liquid. A 14% (w/w) rOVA liquid comprising rOVA, water, and cream of tartar or a 14% (w/w) native egg white liquid comprising chicken egg white, water and cream of tartar were each whisked to generate stiff peaks. The sugar liquid was whisked into the stiff peaks to form the meringues. The meringues were gradually combined with an almond paste (comprising rOVA liquid or egg white liquid, super fine almond flour, and powdered sugar) and mixed until a batter having the desired consistency was achieved. The batter was then piped into equal diameter discs and was allowed to air dry for approximately 25 minutes. The dried batter was then baked for 11 minutes at 275° F., thereby forming the shells.

Buttercream: Vegan butter was tempered and mixed with powdered sugar, unsweetened coconut milk, salt and vanilla bean paste at low speed to make buttercream having a desired light and airy texture. Buttercream was then allowed to cool in a refrigerator and then piped between two macaron shells.

The resulting macaron shells had 2.6% rOVA w/w (on batter weight basis). The resulting, macarons were tested for textural properties and the results are provided in Table 21 and 22 below. In sensory trials, different applications use different sensory surveys but all include a question regarding overall likeability. To maintain statistical power, this study only considers that summary question. Specifically, sensory surveys ask respondents for overall likability ratings on a Likert scale as shown below in Table 20. A “diff score” was calculated which looks at how samples compare relative to how the same rater judged a control sample (control samples use chicken eggs in the same recipe). The calculation for diff score is provided below:

$${l}_{diffscore} = {l}_{sample} - {I}_{control}$$

Here l refers to likeability after conversion using the numerical encoding above. In one example, the analysis combines the 50% and 100% clipped samples and a two sided Mann Whitney U test was used. The difference between clipped (when the values are combined for both: both 50% and 100% clipped) (449 g) and unclipped (504 g) hardness do not reach significance (p>(i/m)0.05, p=0.69).

TABLE 20
Numeric encoding of similarity scores

| Shown to User | Numeric Encoding |
|---|---|
| Dislike extremely | 0 |
| Dislike very much | 1 |
| Dislike moderately | 2 |
| Dislike slightly | 3 |
| Neither like nor | 4 |
| dislike | |
| Like slightly | 5 |
| Like moderately | 6 |
| Like very much | 7 |
| Like extremely | 8 |

TABLE 21
Macaron textural analysis

| | | Median | | |
|---|---|---|---|---|
| | Level of Clipping: | 0% | 50% | 100% |
| Macaron | Hardness | 504.67 | 534.35 | 387.64 |
| | Sensory Raw | 6.00 | 6.00 | |
| | Sensory Delta | 0.00 | 0.00 | |

### Example 18: Pound Cake

This example discloses pound cakes made with ovalbumin as the only egg-white protein. The three protein powders: 100% full-length, 50% full-length (50% clipped) and 100% clipped ovalbumin protein powders were tested against each other for egg-white like properties. Table 22 below provides the formulation of both recipes of pound cake.

TABLE 22
Pound cake ingredients

| Ingredients | Amounts |
|---|---|
| Cake Flour | 25.73 |
| Granulated Sugar | 24.7 |
| Shortening | 4.37 |
| Emulsifier | 1.03 |
| Baking Powder | 1.50 |
| Salt | 0.31 |
| Ovalbumin | ~3.8 * |
| Extra Water for Egg Step | 16.36 |
| Xanthan Gum | 0.10 |
| Calcium Carbonate | 0.25 |
| Calcium Propionate | 0.13 |
| Sodium Acid Pyrophosphate | 0.31 |
| Monocalcium Phosphate | 0.210 |
| Com Syrup | 4.49 |
| Vanilla Extract | 0.500 |
| Vegetable Oil | 5.14 |
| Water | 10.99 |
| Total | 100.00 |

* The weight of the egg white powder and rOVA were adjusted based on their % protein content to deliver 3.3% protein.

The following procedure was used for producing the pound cakes:
a) In a mixing bowl, cream shortening and emulsifier.
b) Add sugar and mix for 3 minutes at medium speed.
c) Put in the dry mix powder ingredients and mix 1 minute at low speed.
d) Mix for 4 minutes medium speed.
e) Check that the batter is homogenous.
f) Add liquid ingredients and mix for 2 minutes at medium speed.
g) Measure batter density, it must be between 0.9-1.0 g/mL.
h) Grease the pan.
i) Pour batter into the pan, approximate weight of batter was 290 g.
j) Bake for 30 minutes at (350 F) or until done.

The resulting pound cakes were measured for textural properties. Tables 23 and 24 provide results from the analysis. Statistical analysis was performed similar to the analysis described in Example 17.

TABLE 22
Textural results of pound cake

| | | Median | | |
|---|---|---|---|---|
| | Level of Clipping: | 0% | 50% | 100% |
| Pound Cake | Center Height | 61.98 | 64.45 | 65.62 |
| | Hardness | 1448.70 | 1317.87 | 1339.31 |
| | Resilience | 0.32 | 0.30 | 0.31 |
| | Cohesiveness | 0.70 | 0.68 | 0.68 |
| | Springiness | 0.85 | 0.87 | 0.86 |
| | Gumminess | 101.45 | 89.58 | 90.95 |
| | Chewiness | 85.43 | 76.90 | 78.40 |
| | Sensory Raw | 5.00 | 6.00 | 6.00 |
| | Sensory Delta | 0.00 | 0.00 | 0.00 |

TABLE 23
Textural results of pound cake

| | Clipped | Undipped | p |
|---|---|---|---|
| Center Height | 64.9 | 62.0 | 0.13 |
| Hardness (g) | 1339.3 | 1448.7 | 0.008 |
| Resilience | 0.31 | 0.32 | 0.09 |
| Cohesiveness | 0.68 | 0.70 | 0.01 |
| Springiness | 0.86 | 0.85 | 0.13 |
| Gumminess | 90.6 | 101.5 | 0.001 |
| Chewiness | 78 | 85 | 0.007 |

### Example 19: Egg White Scramble

An egg white scramble was made using the three protein powders: 100% full-length, 50% full-length (50% clipped) and 100% clipped ovalbumin; formulation is provided in Table 24 below. The three protein powders were tested against each other for egg-white like properties.

TABLE 24
Scramble ingredients

| Ingredients | | % |
|---|---|---|
| Ovomucoid (min.80% protein) | Dry Mix | 6.50 |
| Ovalbumin (min.80% protein) | Dry Mix | 0.88 |
| Kala namak | Dry Mix | 0.70 |
| Curcumin Powder (Color) | Fat/Oil Mix | 0.01 |
| Canola oil | Fat/Oil Mix | 3.90 |
| Soy Lecithin | Fat/Oil Mix | 0.70 |
| High acyl Gellan gum | Gum Mix | 0.60 |
| Gum mix | Gum Mix | 1.70 |
| Psyllium Husk | Gum Mix | 0.50 |
| Pineapple yellow | Water Mix | 0.07 |
| Organic Tapioca syrup DE27 | Water Mix | 0.50 |
| Filtered Water | Water Mix | 83.94 |

Liquid whole egg formula preparation instructions that were followed:
1. Weigh out Dry Mix Ingredients: ovomucoid, ovalbumin, and Kala Namak
2. Weight out Water Mix Ingredients: Filtered Water, Pineapple Color, and Tapioca Syrup
3. Weight out Gum Mix Ingredients: Gellan Gum, Curdlan Gum, and Psyllium Husk
4. Using a Kitchen Aid 4.5 qt Mixer with paddle attachment, add Dry Mix and Water Mix and mix at Speed 1 for 5 minutes till dissolution. Let sit for an additional 10 minutes to hydrate.
5. Add Gum Mix and mix for 5 minutes. Allow it to hydrate for an additional 15 minutes.
6. Prepare Oil Mix with a Norpro Mixer. First add in curcumin powder (colorant) and Canola oil. Mix until the curcumin powder is dispersed in the oil. Then add soy lecithin and use a Norpro Mixer to mix until the content is homogeneous.
7. Add the homogeneous Oil Mix to the KitchenAid 4.5 qt mixer containing the rest ingredients and mix at speed 1 for 2 minutes.
8. Transfer the content to a 12 oz clear Plastic Bottle.

### Cooking Instructions:

Note: 3 Tablespoons=1 Egg (45-50 g)

Scramble cooking as followed:
1. Shake the bottle well before use.
2. Preheat an 8-inch skillet (non-stick) to medium heat (350° F.).
3. Coat the skillet evenly with plant-based butter or oil.
4. Pour 6 tbsp (2 large eggs) of the liquid formula into the skillet.
5. Leave the liquid unstirred till bubbles are observed (˜15-30 seconds), then use a rubber spatula to occasionally stir and scrape and pull mixture across the skillet.
6. Scramble for 2-3 min, ensuring the eggs no longer appear liquid.
7. Allow to cool for 1-2 min before serving.

The resulting egg scrambles were measured for textural properties. Tables 25 and 26 provide results from the analysis. Statistical analysis was performed similar to the analysis described in Example 17.

TABLE 25
Sensory results

| | | Median | | |
|---|---|---|---|---|
| | Level of Clipping: | 0% | 50% | 100% |
| Scramble | Sensory Raw | 2.50 | 6.00 | 7.00 |
| | Sensory Delta | −4.50 | −1.00 | 0.00 |

TABLE 26
Textural results for clipped OVA (50% and 100%) and undipped OVA

| | Clipped | Undipped |
|---|---|---|
| Firmness | 11987 | 10384 |
| Instant | 0.29 | 0.30 |
| Springiness | | |
| Resilience | 0.33 | 0.34 |
| Toughness | 6130 | 5383 |

### Example 20: Burger Binding

A burger was made using the three different protein powders: 10000 full-length, 5000 full-length (50% clipped) and 100% clipped ovalbumin; formulation is provided in Table 27 below. The three protein powders were tested against each other for egg-white like properties.

TABLE 27
Burger Ingredients

| Ingredients | Ingredients |
|---|---|
| Response 4400 Textured Soy | 23.00 |
| Ovalbumin | 4.00 |
| Methylcellulose | 0.00 |
| Onion Powder | 0.50 |
| Garlic Powder | 0.30 |
| Red Beet Powder | 0.50 |
| Salt | 1.00 |
| Yeast Extract | 1.00 |
| Xanthan Gum | 0.50 |
| Modified Potato Starch Penbind 1015 | 1.00 |
| Corn Starch Novation Prima 340 | 1.00 |
| Organic Coconut Oil Refined | 5.75 |
| Canola Oil | 5.75 |
| Liquid Soy Lecithin | 0.50 |
| Water | 55.20 |
| Total | 100.00 |

Mixing steps that were followed:
1. For Control 1, weigh out all of the dry ingredients into a plastic disposable cup. For Control 2 and Test, weigh out all the dry ingredients except for the protein binders (EWP or P2) into a plastic disposable cup.
2. Weigh the protein binders (EWP or P2) separately in a glass beaker.
3. Weigh the required amount of water into the same beaker and dissolve protein with a metal spatula.
4. After manually dissolving the protein in water, put a magnetic stir bar into the beaker and turn on the stir plate at 350 rpm for 5 minutes.
5. Allow the protein to hydrate for at least 30 minutes.
6. Add beet powder and dissolve thoroughly.
7. Hydrate the textured soy protein with the solution and allow it to hydrate for 30 minutes.
8. Using the Kitchen Aid mixer, mix the hydrated textured soy protein for 1 minute at stir speed.
9. Add the dry ingredients and mix for 1 minute at stir speed.
10. Weigh the coconut oil into a glass beaker and either microwave (around 20 seconds) or use the hot plate stirrer at 150° F. for around 2 minutes or until coconut oil is melted.
11. Add melted coconut oil, canola oil, and soy lecithin to the mixing bowl and mix for 1 minute at stir speed.
12. Mold burgers by hand into 30 g burgers, approximately 50 mm diameter×14 mm thickness. Use calipers to confirm dimensions.
13. Freeze the burgers overnight.

### Cooking:

1. Preheat the induction cooktop to 340° F. (wait about 10 minutes or until temperature reaches 340° F., check with a digital thermometer).
2. Use a small amount of canola oil to coat the pan.
3. Cook patty for 5 minutes, then flip to the other side and cook for another 5 minutes.
4. Check the internal temperature of the patty after cooking, should be 165° F.
5. Rest the patties for 5 minutes before taking TPA.
6. For moisture analysis, use a small coffee grinder to grind around 10 g of patty.
7. Take water activity and moisture content reading.

The resulting egg scrambles were measured for textural properties. Table 27 and 28 provide results from the analysis. Statistical analysis was performed similar to the analysis described in Example 17.

Clipped material sees significantly different hardness and chewiness for clipped versus unclipped material in burger binding (p<(i/m)0.05).

TABLE 27
Textural analysis

| | | Median | | |
|---|---|---|---|---|
| | Level of Clipping: | 0% | 50% | 100% |
| Burger Binding | Hardness | 3358.26 | 4097.63 | 4683.91 |
| | Cohesiveness | 0.18 | 0.16 | 0.19 |
| | Springiness | 0.54 | 0.61 | 0.55 |
| | Chewiness | 28.03 | 42.28 | 51.18 |

TABLE 28
Textural results for clipped OVA (50% and 100%) and undipped OVA

| | Clipped | Undipped | P |
|---|---|---|---|
| Hardness (g) | 4274 | 3358 | 0.02 |
| Cohesiveness | 0.19 | 0.16 | 0.17 |
| Springiness | 0.60 | 0.54 | 0.17 |
| Chewiness | 47 | 28 | 0.02 |

### Example 21: Methods of Modulating Recombinant Clipped Ovalbumin (rcOVA)

In one example, recombinant OVA (rOVa) may be produced by a host cell. The host cell may be grown in a fermentation medium where the rOVA would be secreted. In such examples, a protease native to the host cell may clip the rOVA and produce rcOVA. The protease's activity may be increased by modifying the fermentation conditions that are suited for the protease's activity.

In another example, the protease may be overexpressed in the host cell, for example by genetically modifying the host cell. Additional copies of the protease may be expressed in the host cell.

In another example, one or more exogenous proteases may be added to the fermentation medium to increase production of rcOVA.

In yet another example, a protease may be added during purification of the protein, for instance, wherein the fermentation medium is separated from cells and the cell debris before drying.

### Example 22: Methods of Modulating Recombinant Clipped Ovalbumin (rcOVA)

In one example, recombinant OVA (rOVa) may be produced by a host cell. The host cell may be grown in a fermentation medium where the rOVA would be secreted. In such examples, a protease native to the host cell may clip the rOVA and produce rcOVA. The protease's activity may be decreased by modifying the fermentation conditions that are not suited for the protease's activity.

In another example, the protease may be underexpressed in the host cell, for example by genetically modifying the host cell. Copies of the protease may be knocked out from the host cell.

In another example, one or more exogenous protease inhibitors may be added to the fermentation medium to decrease production of rcOVA.

In another example, one or more exogenous protease inhibitors may be expressed or overexpressed in the host cell.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A consumable composition comprising a recombinant ovalbumin (rOVA) protein and a recombinant clipped ovalbumin (rcOVA) protein;
a. wherein the rOVA protein comprises a single polypeptide molecule;
b. wherein the rcOVA protein comprises a complex of two or more polypeptide molecules; and
c. wherein an ovalbumin content of the consumable composition comprises the rOVA protein and the rcOVA protein, with the rcOVA protein comprising at least 0.1% w/w of the ovalbumin content.

2. The consumable composition of claim 1, wherein the two or more polypeptide molecules of the rcOVA protein are connected via non-covalent bonds.

3. The consumable composition of claim 2, wherein the two polypeptide molecules are connected in a configuration similar to a configuration of a native ovalbumin (nOVA) protein or in a configuration similar to a configuration of the rOVA protein.

4. The consumable composition of claim 1, wherein the two or more polypeptide molecules of the rcOVA protein together have an amino acid sequence with at least 95% sequence identity to the rOVA protein or to a native ovalbumin (nOVA) protein.

5. The consumable composition of claim 4, wherein the two or more polypeptide molecules of the rcOVA protein together have an amino acid sequence with at least 99% sequence identity to rOVA or to nOVA or have an amino acid sequence with 100% identity to rOVA or to nOVA.

6. The consumable composition of claim 1, wherein the rcOVA protein has the same number of amino acids as the rOVA protein or as a native ovalbumin (nOVA) protein.

7. The consumable composition of claim 1, wherein the rcOVA protein is clipped at a serine protease cleavage site.

8. The consumable composition of claim 7, wherein the cleavage site is selected from the group consisting of Ala352-Ser353, Asp350-Ala351, and His22-Ala23 with respect to SEQ ID NO: 75.

9. The consumable composition of claim 1, wherein the rcOVA protein comprises at least 0.5% w/w of the ovalbumin content.

10. The consumable composition of claim 1, wherein the rcOVA protein comprises at most 70% w/w of the ovalbumin content.

11. The consumable composition of claim 1, wherein the consumable composition is a powdered ingredient composition.

12. The consumable composition of claim 11, wherein the ovalbumin content comprises at least 85% w/w of the powdered ingredient composition.

13. The consumable composition of claim 1, wherein the consumable composition is a food product.

14. The consumable consumption of claim 13, wherein the food product has a texture different from a texture of a control food product, wherein the control food product is substantially identical to the food product except the control food product lacks the rcOVA protein and comprises rOVA or lacks the rcOVA protein and comprises a native ovalbumin (nOVA) protein.

15. The consumable composition of claim 13, wherein the food product has a hardness different from a hardness of a control food product, wherein the control food product is substantially identical to the food product except the control food product lacks the rcOVA protein and comprises rOVA or lacks the rcOVA protein and comprises a native ovalbumin (nOVA) protein.

16. The consumable composition of claim 13, wherein the ovalbumin content comprises at least 1% w/w of the food product.

17. The consumable composition of claim 13, wherein the ovalbumin content comprises from about 2% to about 15% w/w of the food product.

18. The consumable composition of claim 13, wherein the food product is a baked food product.

19. The consumable composition of claim 13, wherein the food product is a cooked food product.

20. The consumable composition of claim 13, wherein the food product comprises one or more additional recombinant egg white proteins.

21. The consumable composition of claim 1, wherein the consumable composition is a liquid composition.

22. The consumable composition of claim 21, wherein the rOVA protein comprises at least 50% w/w of the total protein in the liquid composition or at least 50% w/w of the liquid composition.

23. The consumable composition of claim 21, wherein the pH of the liquid composition is from about 3.5 to about 10.

24. The consumable composition of claim 1, wherein the rOVA protein further includes an EAEA amino acid sequence (SEQ ID NO: 76) at its N-terminus.

25. The consumable composition of claim 1, wherein the rcOVA protein further includes an EAEA amino acid sequence (SEQ ID NO: 76) at the N-terminus of one of its two or more polypeptide molecules.

26. The consumable composition of claim 1, wherein the rOVA protein is expressed by a yeast host cell.

27. The consumable composition of claim 26, wherein the host cell is selected from a Pichia species, and a Saccharomyces species.

28. The consumable composition of claim 1, wherein the rOVA protein is expressed by a fungal host cell.

29. The consumable composition of claim 28, wherein the host cell is selected from a Trichoderma species, and an Aspergillus species.

30. The consumable composition of claim 1, wherein the rcOVA protein is expressed by a first host cell and the rOVA protein is expressed by a second host cell or wherein the rcOVA protein and the rOVA protein are expressed by the same host cell.


SEQUENCE LISTING (75 sequences)

SEQ ID NO: 1 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 2 - protein, 389 aa, synthetic construct
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 3 - protein, 419 aa, synthetic construct
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 4 - protein, 406 aa, synthetic construct
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 5 - protein, 451 aa, synthetic construct
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 6 - protein, 386 aa, synthetic construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 7 - protein, 386 aa, synthetic construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 8 - protein, 417 aa, synthetic construct
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 9 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 10 - protein, 386 aa, synthetic construct
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 11 - protein, 411 aa, synthetic construct
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 12 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 13 - protein, 408 aa, synthetic construct
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 14 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 15 - protein, 386 aa, synthetic construct
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 16 - protein, 386 aa, synthetic construct
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 17 - protein, 386 aa, synthetic construct
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 18 - protein, 386 aa, synthetic construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 19 - protein, 386 aa, synthetic construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 20 - protein, 386 aa, synthetic construct
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 21 - protein, 386 aa, synthetic construct
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 22 - protein, 386 aa, synthetic construct
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 23 - protein, 386 aa, synthetic construct
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 24 - protein, 386 aa, synthetic construct
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 25 - protein, 386 aa, synthetic construct
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 26 - protein, 386 aa, synthetic construct
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 27 - protein, 391 aa, synthetic construct
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 28 - protein, 386 aa, synthetic construct
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 29 - protein, 386 aa, synthetic construct
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 30 - protein, 386 aa, synthetic construct
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 31 - protein, 386 aa, synthetic construct
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 32 - protein, 385 aa, synthetic construct
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 33 - protein, 386 aa, synthetic construct
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 34 - protein, 386 aa, synthetic construct
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 35 - protein, 386 aa, synthetic construct
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 36 - protein, 385 aa, synthetic construct
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 37 - protein, 386 aa, synthetic construct
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 38 - protein, 386 aa, synthetic construct
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 39 - protein, 386 aa, synthetic construct
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 40 - protein, 386 aa, synthetic construct
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 41 - protein, 386 aa, synthetic construct
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 42 - protein, 386 aa, synthetic construct
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 43 - protein, 386 aa, synthetic construct
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 44 - protein, 386 aa, synthetic construct
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 45 - protein, 386 aa, synthetic construct
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 46 - protein, 381 aa, synthetic construct
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 47 - protein, 391 aa, synthetic construct
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 48 - protein, 383 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 49 - protein, 386 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 50 - protein, 392 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 51 - protein, 386 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 52 - protein, 395 aa, synthetic construct
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 53 - protein, 386 aa, synthetic construct
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 54 - protein, 386 aa, synthetic construct
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 55 - protein, 385 aa, synthetic construct
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 56 - protein, 386 aa, synthetic construct
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 57 - protein, 383 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 58 - protein, 386 aa, synthetic construct
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 59 - protein, 386 aa, synthetic construct
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 60 - protein, 2174 aa, synthetic construct
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 61 - protein, 386 aa, synthetic construct
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 62 - protein, 2104 aa, synthetic construct
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 63 - protein, 386 aa, synthetic construct
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 64 - protein, 420 aa, synthetic construct
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 65 - protein, 386 aa, synthetic construct
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 66 - protein, 386 aa, synthetic construct
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 67 - protein, 386 aa, synthetic construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 68 - protein, 386 aa, synthetic construct
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 69 - protein, 386 aa, synthetic construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 70 - protein, 399 aa, synthetic construct
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 71 - protein, 389 aa, synthetic construct
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 72 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 73 - protein, 389 aa, synthetic construct
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 74 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 75 - protein, 396 aa, synthetic construct
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPNORMALDOTM